(19) United States
(12) Patent Application Publication
(10) Pub. No.: US 2025/0131997 A1
(43) Pub. Date: Apr. 24, 2025

(54) SYSTEMS AND METHODS FOR AUTOMATED MEDICAL DATA CAPTURE AND CAREGIVER GUIDANCE

(71) Applicant: ZOLL Medical Corporation, Chelmsford, MA (US)

(72) Inventors: Frederick W. Forester, Encinitas, CA (US); Corissa J. Bowman, Golden, CO (US); Keenan S. Early, Denver, CO (US); Peter G. Goutmann, Gibsonia, PA (US); Rainer Grote, Denver, CO (US); Matthew R. Vawter, Boulder, CO (US); Stephen A. Frye, Broomfield, CO (US)

(21) Appl. No.: 18/681,542

(22) PCT Filed: Aug. 5, 2022
(86) PCT No.: PCT/US2022/074596

§ 371 Date: Feb. 6, 2024

Related U.S. Application Data
(60) Provisional application No. 63/230,393, filed on Aug. 6, 2021.

(51) Int. Cl. G16H 10/65; G16H 15/00; G16H 50/20

(52) U.S. Cl. CPC G16H 10/65; G16H 15/00; G16H 50/20

(57) ABSTRACT

A patient data charting device configured to automatically capture electronic patient care record (ePCR) data from a caregiver is provided. The device includes a memory storing an ePCR including a plurality of data fields, an output device, a microphone configured to acquire speech, and a processor. The processor is configured to convert the speech to text, identify a first value of a data field of the plurality of data fields based on the text, populate the first data field with the first value, generate a prompt that requests a second value of a second data field of the plurality of data fields based on the first data field, and present the prompt to the caregiver via the output device.

### CROSS REFERENCE TO RELATED APPLICATIONS

This application claims priority under 35 U.S.C. § 119(e) to U.S. Provisional Application Ser. No. 63/230,393, titled “SYSTEMS AND METHODS FOR AUTOMATED MEDICAL DATA CAPTURE AND CAREGIVER GUIDANCE,” filed Aug. 6, 2021, which is hereby incorporated herein by reference in its entirety.

### BACKGROUND

Emergency medical services (EMS) agencies create and use an electronic patient care record (ePCR) for each patient encounter. The ePCR contains a complete record of medical observations and treatments for the patient during the patient encounter. The ePCR includes times for the observations and treatments, patient medical history information, and transport information (e.g., from a scene of an emergency to a medical care facility). Based in part on the complexities of medical diagnosis and care in these situations along with governmental reporting guidelines, the ePCR may be typically a complex and lengthy document.

Software applications exist that interact with EMS personnel to complete ePCRs. These software applications include user interface screens with controls to receive input from EMS personnel regarding a patient encounter. This input specifies values of data fields that document the complete record of medical observations and treatments described above.

In the pre-hospital and/or acute care treatment setting, medical responders often have difficulty in accurately determining the most effective medical interventions for a patient. In these settings, split second decisions about interventions for emergency conditions such as respiratory distress, cardiac arrest, and/or trauma are often required based on a minimal amount of information about a patient.

To alleviate these difficulties, rescuers can benefit from tools that guide care through automated recordation. Information about physiologic data, interventions delivered to the patient, and the patient's health history and status may be collected by sensors and from various databases. Such information may be integrated, analyzed, and recorded in an automated fashion to provide life-saving guidance for effective and immediate medical interventions.

### SUMMARY

In one example, a patient data charting device is provided. The patient charting device is configured for automatically capturing electronic patient care record (ePCR) data from a caregiver. The device includes a memory storing an ePCR including a plurality of data fields; at least one output device; a microphone configured to acquire speech regarding a patient encounter; and at least one processor. The at least one processor is configured to execute operations to convert the speech to text, identify at least one first value of at least one first data field of the plurality of data fields based on the text, populate the at least one first data field with the at least one first value, generate at least one prompt that requests at least one second value of at least one second data field of the plurality of data fields based on the at least one first data field, and present the at least one prompt to the caregiver via the at least one output device.

Examples of the patient data charting device can include one or more of the following features.

In the patient data charting device, the at least one processor may be configured to execute operations to identify the at least one second data field based on an organizational structure of the ePCR. The organizational structure of the ePCR may include data field sections organized according to medical procedure categories and/or medical condition categories. The data field section may include one or more of a dispatch section, a patient assessment section, or a respiratory/cardiac section. The at least one processor may be configured to execute operations to identify the at least one second data field as being procedurally related to the at least one first data field and generate the at least one prompt in response to the identification of the procedural relationship. The procedural relationship may correspond to a relationship between steps in an iterative diagnosis procedure based on a patient's presentation. The at least one first data field may include one of observation data, intervention data, physiological sensor data, and diagnosis data, and the at least one second data field may include at least one other of the observation data, the intervention data, the physiological sensor data, and the diagnosis data related to the at least one first data field. The at least one first data field and the at least one second data field may be procedurally related by being associated with a same treatment protocol. The same treatment protocol may be defined within at least one of a diagnostic sequence of activities and/or data entry and an intervention sequence of activities and/or data entry.

In the patient data charting device, the at least one processor may be configured to execute the operations through execution of a digital assistant. The at least one output device may include at least one of a speaker coupled to the at least one processor and a touchscreen coupled to the at least one processor, and the digital assistant may be configured to render the one or more prompts via one or more of the speaker or the touchscreen.

The patient data charting device may further include a camera configured to acquire images, and the digital assistant may be configured to process the images to record one or more of an identifier of medication from a medication label, handwritten text, electrocardiogram (ECG) information from an ECG tape and/or a screen shot of a medical device display, driver's license information, insurance card information, or patient information from a face sheet.

In the patient data charting device, the identifier of the medication may be a quick response code. The digital assistant may be further configured to identify, based on the text, a first physiologic sensor that generated the at least one first value; convert additional speech to additional text; identify at least one third value of the at least one first data field based on the additional text; identify, based on the additional text, a second physiologic sensor that generated the at least one third value; identify the second physiologic sensor as being a sensor of record for the at least one first data field based on a clinically derived sensor preference; and replace the at least one first value in the at least one first data field with the at least one third value.

In the patient data charting device, the digital assistant may be further configured to operate in two or more of a plurality of interactivity modes and switch from a first interactivity mode to a second interactivity mode based on additional speech. The plurality of interactivity modes may include two or more of a user-driven mode in which the digital assistant is configured to follow express commands of the caregiver articulated in the additional speech; a predictive mode in which the digital assistant is configured to autonomously navigate to one or more sections of the ePCR procedurally related to a data field of the plurality of data fields referenced in the additional speech; a confirmation mode in which the digital assistant is configured to prompt the caregiver to confirm values of data fields referenced in the additional speech prior to population of the data fields with the values; an observational mode in which the digital assistant is configured not to prompt the caregiver to confirm the values of the data fields referenced in the additional speech prior to population of the data fields with the values; and a conversational mode in which the digital assistant is configured to prompt the caregiver for additional values of additional data fields procedurally related to a data field of the plurality of data fields referenced in the additional speech.

In the patient data charting device, the digital assistant may include a locally executed natural language processor configured to convert the unstructured text to structured text. The speech may include language directed to one or more of a patient, a caregiver, a bystander, or another device. The natural language processor may be trained to identify, within communications articulated in a human language, data elements defined in an ePCR standard.

The ePCR standard may be one or more of a National Emergency Medical Service Information System (NEMSIS) standard or a Fast Healthcare Interoperability Resources (FHIR) standard. To identify the at least one first value of the at least one first data field may include to identify, via the natural language processor, an intent in the text to document a value of a data element defined in the ePCR standard, extract, via the natural language processor, a first slot value from the text that specifies an identifier of the data element, extract, via the natural language processor, a second slot value from the text that specifies a value of the data element, and map the identifier of the data element to an identifier of the at least one first data field; and to populate the at least one first data field may include to convert the value of the data element to the at least one value. The digital assistant may be further configured to determine whether the value of the data element is valid according to the ePCR standard.

In the patient data charting device, the at least one processor may be configured to identify the at least one second data field as being procedurally related to the at least one first data field based on a predictive workflow. The predictive workflow may identify procedurally related fields based on one or more of a geolocation, an EMS transport mode, a type of EMS service, and a medical protocol. The EMS transport mode may include a medivac service or an ambulance service. The type of EMS service may include a scheduled call or an emergency call. The type of EMS service may include a medical emergency identification from a dispatch service. The predictive workflow may be customizable by an EMS organization.

In the patient data charting device, the device may include one or more of a smartphone, a tablet, a portable computing device, a wearable computing device, and combinations thereof. The patient data charting device may further include a network interface coupled to the at least one processor and configured to communicably couple to at least one distinct device via the network interface. In the patient data charting device, the at least one distinct computing device may include a medical device and the at least one processor may be further configured to receive, via the network interface a medical device identifier transmitted from the medical device; and store the medical device identifier with the ePCR. The at least one distinct computing device may include a medical device and the at least one processor may be further configured to receive, via the network interface, a summary report transmitted from the medical device and including at least one of patient treatment information and patient physiologic information; identify at least one third value for at least one third data field from the summary report; and populate the at least one third data field with the at least one third value. The at least one processor may be further configured to identify unfilled data fields in the stored ePCR, transmit the stored ePCR and information indicative of the unfilled data fields to a cloud server accessible by the distinct computing device via the network interface, and the at least one distinct computing device may have a larger form factor than the patient data charting device. The distinct computing device may include a tablet computer, a laptop computer, and/or an edge server.

The patient data charting device may further include a network interface coupled to the at least one processor and configured to communicate with a remote server, the at least one processor being further configured to generate a quick response (QR) code; associate the QR code with the stored ePCR; and transmit the QR code with the stored ePCR to the remote server via the network interface. The remote server may be configured to receive the transmitted QR code and ePCR; store the transmitted ePCR at the remote server; and store the QR code as a pointer to the transmitted ePCR stored at the remote server. The remote server may be an edge server located in mobile computing environment or a cloud server located in a cloud environment. The caregiver may include one or more of an emergency medical technician, a paramedic, a medic, a physician, a nurse, and a medical scribe.

In another example, a patient data charting device is provided. The patient data charting device is configured for automatically capturing electronic patient care record (ePCR) data from a caregiver. The device includes a memory storing an ePCR including a plurality of data fields, the plurality of data fields including at least one first ePCR data field; at least one user interface device configured to receive input including unstructured data corresponding to a human language communication regarding a patient encounter; and at least one processor. The at least one processor is configured to execute operations to identify at least one first ePCR data field corresponding to the unstructured data, transform at least a portion of the unstructured data to structured data including at least one data field value based on a validation requirement for the at least one first data field, and populate the at least one first data field in the ePCR with the structured data.

Examples of the patient data charting device can include one or more of the following features.

In the patient data charting device, the at least one user interface device may include a microphone and the at least one processor may be configured to transform the unstructured data based on a speech-to-text conversion from at least a first portion of the input received via the microphone. The at least one user interface device may include one or more of a touchscreen, a scanner, a camera, a keyboard, and a virtual reality device. The validation requirement may include at least one of a data field format requirement and a data field rule. To identify the at least one first ePCR data field corresponding to the unstructured data may include to identify, using at least one natural language processor, at least one intent expressed within the unstructured data to document at least one value of at least one data element defined in an ePCR standard; and extract at least one slot value from the unstructured data that specifies an identifier of the at least one first data field. The at least one user interface device may further include a speaker and the at least one processor may be configured to identify at least one second ePCR data field as being procedurally related to the at least one first ePCR data field based on a workflow for the ePCR, generate at least one prompt associated with the at least one second ePCR data field, and present the at least one prompt to the caregiver via the speaker and the touchscreen. The workflow may be a predictive workflow.

In the patient data charting device, the at least one processor may be configured to identify a context for natural language processing based on the unstructured data, and select the predictive workflow based on the identified context. The context may correspond to one or more EMS interventions and procedures. The predictive workflow may provide an order of population for fields of the ePCR based on one or more of a medical care protocol, historic medical outcomes, and an observed order of population for fields of the ePCR. The predictive workflow may be customizable by an EMS organization. The at least one prompt may include a request for input corresponding to at least one second value for the at least one second ePCR data field. The at least one prompt may include one or more of an instruction, a reminder, and an alarm corresponding to an intervention associated with the at least one second ePCR data field. The at least one prompt may include a request for confirmation of the at least one data field value prior to population of the at least one first ePCR data field. The at least one first ePCR data field and the at least one second ePCR data field may correspond to different sections of the ePCR.

The patient data charting device may further include a camera configured to acquire images, and the at least one processor may be configured to process the images to record one or more of an identifier of medication from a medication label, electrocardiogram (ECG) information from an ECG tape and/or a screen shot of a medical device display, driver's license information, insurance information, or patient information from a face sheet. The patient data charting device may further include a camera configured to acquire images of handwritten text, and the at least one processor may be configured to process the images to generate the unstructured data from the handwritten text.

In the patient data charting device, the images of handwritten text may include images of handwritten text on a medical glove. The at least one processor may be configured to identify a context of the handwritten text, identify at least one element of the handwritten text that is inconsistent with the context, and replace the at least one element of the handwritten text with a new element that is consistent with the context. The at least one processor may be configured to transform the at least a portion of the unstructured data to structured text using a locally executed natural language processor configured to convert unstructured text to structured text. The natural language processor may be trained to identify, within communications articulated in a human language, data elements defined in an ePCR standard.

The ePCR standard may be one or more of a National Emergency Medical Service Information System (NEMSIS) standard or a Fast Healthcare Interoperability Resources (FHIR) standard. The at least one processor may be further configured to validate the at least one data field value. The patient data charting device may further include one or more of a smartphone, a tablet, a portable computing device, a wearable computing device, or combinations thereof. The caregiver may include one or more of an emergency medical technician, a paramedic, a medic, a physician, a nurse, and a medical scribe.

In another example, a system for providing digital assistance for automated patient charting by a caregiver is provided. The system includes a memory including an electronic patient care record (ePCR); a user interface configured to interact with the caregiver; and at least one processor coupled to the memory and the user interface. The at least one processor is configured to execute a digital assistant configured to: receive unstructured data from the caregiver; identify at least one data field of the ePCR related to the unstructured data; identify a user interface (UI) control related to the at least one data field of the ePCR; and render, via the user interface, the UI control to the caregiver.

Examples of the system can include one or more of the following features.

In the system, the digital assistant may be configured to transform at least a portion of the unstructured data to structured data based on a validation requirement for the at least one data field. The system may further include a microphone coupled to the at least one processor and configured to acquire an audio signal, and the at least one processor may be configured to derive speech data from the audio signal. The unstructured data may include the derived speech data. In the system, the UI may include a speaker and the digital assistant may be further configured to identify at least one first value of the at least one first ePCR data field; populate the at least one first ePCR data field with the at least one first value; identify at least one second ePCR data field; and prompt the caregiver via a human language communication from the speaker to input at least one second value of the at least one second ePCR data field. The user interface may include a touchscreen and to prompt may include to duplicate the prompts from the speaker at the touchscreen. The digital assistant may be further configured to identify, based on the speech data, a first physiologic sensor that generated the at least one first value; receive additional speech data; identify at least one third value of the at least one first ePCR data field based on the additional speech data; identify, based on the additional speech data, a second physiologic sensor that generated the at least one third value; identify the second physiologic sensor as being a sensor of record for the at least one first ePCR data field based on a clinically derived sensor preference; and replace the at least one first value in the at least one first ePCR data field with the at least one third value. The digital assistant may be further configured to generate a quick response (QR) code; and associate the ePCR with the QR code. The digital assistant may be further configured to receive a medical device identifier; and store the medical device identifier with the ePCR.

In the system, the digital assistant may be further configured to receive a summary report generated by a medical device and including at least one of patient treatment information and patient physiologic information; identify at least one third value for at least one third data field from the summary report; and populate the at least one third data field with the at least one third value. The system may further include a camera configured to acquire images, and the digital assistant may be further configured to process the images to record one or more of an identifier of medication from a medication label, text from handwriting on a glove, electrocardiogram (ECG) information from an ECG tape and/or a screen shot of a medical device display, driver's license information, patient insurance card information, or patient information from a face sheet. In the system, the digital assistant may be further configured to store the acquired images in storage private to the digital assistant. The digital assistant may be further configured to identify a wake-up word in the speech data prior to executing other operations. The digital assistant may be further configured to operate in two or more of a plurality of interactivity modes; and switch from a first interactivity mode to a second interactivity mode based on additional speech data.

In the system, the plurality of interactivity modes may include a user-driven mode in which the digital assistant is configured to follow express commands in the additional speech data. The express commands may include one or more of a command to navigate to a specific UI control within the user interface or a command to store values in ePCR data fields. The plurality of interactivity modes may include a predictive mode in which the digital assistant is configured to autonomously navigate to one or more UI controls within the user interface based on the additional speech data. The one or more UI controls may be associated with one or more ePCR data fields and, while in predictive mode, the digital assistant may be further configured to prompt the caregiver for at least one value of at least one ePCR data field related to the one or more ePCR data fields; and populate the at least one ePCR data field with the at least one value. The at least one data field of the ePCR may be within a same organizational section of the ePCR as the one or more ePCR data fields. The same organizational section may include one or more of a dispatch section, a patient assessment section, or a respiratory/cardiac section. The at least one ePCR data field may be related to the one or more ePCR data fields based on an iterative diagnosis procedure corresponding to a patient's presentation.

In the system, the at least one ePCR data field may include one of observation data, intervention data, physiological sensor data, and diagnosis data, and the one or more ePCR data fields may include at least one other of the observation data, the intervention data, the physiological sensor data, and the diagnosis data related to the at least one ePCR data field. The at least one ePCR data field and the one or more ePCR data fields may be associated with a same treatment protocol. The same treatment protocol may be defined within at least one of a diagnostic sequence of activities and/or data entry and an intervention sequence of activities and/or data entry. The one or more UI controls may be within a threshold number of navigation interactions of a UI control associated with an ePCR data field referenced in the additional speech. The plurality of interactivity modes may include a confirmation mode in which the digital assistant is configured to prompt the caregiver to confirm operations identified by the digital assistant prior to execution of the operations. The operations identified by the digital assistant may include one or more of navigation to a specific UI control within the user interface or storage of values in ePCR data fields.

In the system, the plurality of interactivity modes may include an observational mode in which the digital assistant is configured not to prompt the caregiver to confirm operations identified by the digital assistant prior to execution of the operations. The operations identified by the digital assistant include storage of values in ePCR data fields based on one or more of patient information or intervention information articulated in the additional speech. The plurality of interactivity modes may include a conversational mode in which the digital assistant is configured to prompt the caregiver for additional information needed to complete operations identified by the digital assistant. The operations identified by the digital assistant may include storage of values in ePCR data fields for an incomplete section of the ePCR; and to prompt may include to prompt the caregiver for additional values of additional ePCR data fields with a same section as an ePCR data field referenced in the additional speech data. The digital assistant may be further configured to receive, via the user interface, input specifying a default interactivity mode of the plurality of interactivity modes; and operate in the default interactivity mode.

In the system, the digital assistant may be further configured to receive, via the user interface, input specifying a fallback interactivity mode of the plurality of interactivity modes; calculate a chaos score based on the audio signal; and operate in a fallback interactivity mode where the chaos score transgresses a threshold. The digital assistant may include a natural language processor trained to identify, within communications articulated in a human language, data elements defined in an ePCR standard. The ePCR standard may be one or more of a National Emergency Medical Service Information System (NEMSIS) standard or a Fast Healthcare Interoperability Resources (FHIR) standard. The natural language processor may be hosted locally within the system and the system may be a mobile computing device.

In another example, a mobile computing device is provided. The mobile computing device includes a memory storing at least one natural language processor trained to identify intents related to completion of an electronic patient care record (ePCR); a user input device; and at least one processor coupled to the memory and the user input device. The at least one processor is configured to receive unstructured information expressed in human language; identify, using the at least one natural language processor, an intent expressed within the unstructured information to document at least one value of at least one data element defined in an ePCR standard; and store, in the memory and responsive to identification of the intent, the at least one value in association with an identifier of the at least one data element.

Examples of the mobile computing device can include one or more of the following features.

In the mobile computing device, the user input device may include a microphone and the at least one processor may be configured to receive the unstructured information as an audible utterance, render the audible utterance as text using an automated speech recognition (ASR) engine, and identify the intent expressed within the text. The user input devices may include a keyboard or a touch screen and the at least one processor may be configured to receive the unstructured information as typed text input and identify the intent expressed within the text. The ePCR standard may be one or more of a National Emergency Medical Service Information System (NEMSIS) standard or a Fast Healthcare Interoperability Resources (FHIR) standard. To store the at least one value may include to extract, via the at least one natural language processor, a first slot value from the text that specifies an identifier of the data element; and extract, via the at least one natural language processor, a second slot value from the text that specifies a value of the data element. The at least one processor may be further configured to determine whether the value of the data element is valid according to the ePCR standard. The memory may store an ePCR including a plurality of fields and the at least one processor may be further configured to map the identifier of the data element to a data field of the plurality of fields; and populate the data field with the value of the data element. The at least one processor may be further configured to transform the value of the data element to generate a transformed value, wherein to populate the data field includes to populate the data field with the transformed value. The at least one natural language processor may be trained using textual structures used by caregivers. The caregivers may include EMS personnel. The caregivers may include a medic, a physician, a nurse, and a medical scribe.

In the mobile computing device, the textual structures used by the caregivers may include individual sentences that include one or more slot values that specify identifiers of data elements defined in the ePCR standard and one or more slot values that specify values for the data elements. The one or more slot values may include, for example, at least one slot value, at least two slot values, at least three slot values, or four or more slot values. In some examples, the number of slot values may vary with the information density of the textual structures. The textual structures may be constructed using the data elements defined in the ePCR standard and valid values of the data elements. The textual structures may be specific to one or more of a period of time, a location of caregivers, and a type of medical service conducted by the caregivers. The type of medical service may include emergency medical care in a mobile environment, medical care in a mobile environment, or non-emergency medical transport. The at least one natural language processor may include a plurality of natural language processors trained using a plurality of training data sets. The plurality of training data sets may include a context data set and a section data set for each section in the ePCR standard. The intent may include an intent to document one or more of patient data, a task undertaken by a caregiver, or information required by a section of the ePCR. The intent may include an intent to control operation of the mobile computing device. The intent to control operation of the mobile computing device may include one or more of an intent to navigate to an identified user interface control or an intent to select a default interactivity mode of a digital assistant. The intent may include an intent to send a communication to a device distinct from the mobile computing device. To identify the intent may include to generate a metric that indicates a confidence that the intent is an actual intent. The at least one processor may be further configured to switch a default interactivity mode of a digital assistant to confirmation mode in response to the metric being less than a threshold value. The at least one processor may be further configured to switch a default interactivity mode of a digital assistant to observational mode in response to the metric being greater than a threshold value.

In the mobile computing device, the at least one processor may be further configured to identify, based on at least one value of the at least one data element, a first source device that generated the at least one value; receive, additional unstructured information expressed in the human language; identify at least one additional value of the at least one data element based on the additional unstructured information; identify, based on the additional unstructured information, a second source device that generated the at least one additional value; identify the second source device as being a device of record for the at least one data element; and store the at least one additional value in association with the identifier of the at least one data element. The at least one natural language processor may be hosted locally within the mobile computing device. The mobile computing device may include an smartphone and/or an edge server communicably coupled with the smartphone via a local area network. The at least one natural language processor may include one or more natural language processors trained using data sourced from one or more of an ePCR standard, a medical device, shorthand terminology, or a customer specific vocabulary.

In another example, a caregiver assistance device for assisting a caregiver providing care to a subject is provided. The caregiver device includes a memory storing one or more caregiver activity sequence models; at least one user input device; an output device for providing prompts to the caregiver; and at least one processor coupled to the memory and the at least one user input device. The at least one processor is configured to receive, from the user input device, unstructured information expressed in human language; identify at least one intent expressed within the unstructured information; identify a position within a sequence of caregiving activities based on the at least one intent and the one or more caregiver activity sequence models; and provide, using the output device, one or more prompts to the caregiver regarding subsequent caregiving activities based on the identified position within the sequence of caregiving activities.

Examples of the caregiver assistance device can include one or more of the following features.

In the caregiver assistance device, the plurality of prompts may relate to probable subsequent activities to be performed by the caregiver. The caregiver assistance device may further include a display output device, the plurality of prompts may be displayed concurrently on the display output device, and the at least one user input device may include a microphone for receiving the human language input. In the caregiver assistance device, the at least one processor may be configured to receive the unstructured information as human language input and record entries concerning the caregiving process in an electronic patient care record based on the human language input. The at least one processor may be configured to calculate a chaos score for the mobile environment, and operate in a plurality of interactivity modes including a default interactivity mode and a fallback interactivity mode; and switch between the default interactivity mode and the fallback interactivity mode automatically based on the chaos score. The at least one processor may be configured to receive an ambient noise signal via the user interface device, calculate the chaos score based on the ambient noise signal, compare the chaos score to a threshold, and automatically switch between the default interactivity mode and the fallback interactivity mode based on the comparison between the chaos score and the threshold. The at least one processor may be configured to delay a delivery of caregiver prompts until the chaos score drops below the threshold. The at least one processor may be configured to identify a context based on the ambient noise signal and provide the one or more prompts based on the identified context. The at least one processor may be configured to generate haptic caregiver prompts while the chaos score exceeds the threshold. The at least one processor may be configured to record audio input and identify the unstructured information from the recorded audio input while the chaos score exceeds the threshold. The at least one processor may be configured to discriminate between the unstructured information and ambient noise. The default interactivity mode may be a conversational mode and the fallback interactivity mode may be an observational mode. The caregiver providing care may include performing a method of treatment or diagnosis on the subject. The caregiver assistance device may be a mobile device, and the at least one processor may operate locally at the caregiver assistance device.

In another example, a caregiver assistance device for assisting a caregiver providing care to a subject is provided. The caregiver assistance device includes a memory storing natural language processor (NLP) models including a general NLP model and a plurality of caregiving context-specific NLP models; at least one user input device; and at least one processor coupled to the memory and the at least one user input device. The at least one processor is configured to receive, from the user input device, human language input; identify, using the general NLP model, at least one intent regarding a type of care to be administered to the subject expressed within the human language input; and invoke, for processing subsequent human language input, at least one of the plurality of caregiving context-specific NLP models based on the type of care to be administered.

Examples of the caregiver assistance device can include one or more of the following features.

In the caregiver assistance device, the memory may further store a plurality of caregiver activity sequence models, and each caregiver activity sequence model may be associated with at least one caregiving context-specific NLP model. The at least one processor may be configured to identify a position within a sequence of caregiving activities based on the human language input. The at least one processor may be configured to provide the user guidance based on the invoked at least one model. Assisting the caregiver may include generating a plurality of prompts for the caregiver based on the position within the sequence of caregiving activities, wherein the plurality of prompts relates to probable subsequent activities to be performed by the caregiver.

The caregiver assistance device may further include a display output device, the plurality of prompts may be displayed concurrently on the display output device, and the at least one user input device may include a microphone for receiving the human language input. Assisting a caregiver may include recording, based on the human language input, entries concerning the caregiving process in an electronic subject care record. In the caregiver assistance device, the at least one processor may be configured to calculate a chaos score for the mobile environment, and operate in a plurality of interactivity modes including a default interactivity mode and a fallback interactivity mode; and switch between the default interactivity mode and the fallback interactivity mode automatically based on the chaos score. The at least one processor may be configured to receive an ambient noise signal via the user interface device, calculate the chaos score based on the ambient noise signal, compare the chaos score to a threshold, and automatically switch between the default interactivity mode and the fallback interactivity mode based on the comparison between the chaos score and the threshold. The default interactivity mode may be a conversational mode and the fallback interactivity mode may be an observational mode. The caregiver providing care may include performing a method of treatment or diagnosis on the subject. The caregiver assistance device may be a mobile device, and the at least one processor may operate locally at the caregiver assistance device.

In some examples, an edge server hosts the general NLP model and/or the context-specific NLP models.

In another example, a system for providing digital assistance for an emergency medical services (EMS) record by a user is provided. The system includes a memory including the EMS record; one or more user interface devices configured to interact with the user; and at least one processor coupled to the memory and the one or more user interface devices. The at least one processor is configured to execute a digital assistant configured to receive unstructured data from the user corresponding to a human language communication, identify at least one data field of the EMS record related to the unstructured data, transform at least a portion of the unstructured data to structured data including at least one data field based on a validation requirement for the at least one data field, and populate the at least one data field in the EMS record with the structured data.

Examples of the system can include one or more of the following features.

In the system, the digital assistant may be configured to identify a user interface (UI) control related to the at least one data field in the EMS record, and render, via the one or more user interface devices, the UI control to the user. The EMS record may include an electronic patient care record. The EMS record may include a trip file for EMS dispatch. The EMS record may include a billing record. The EMS record may include a request form for patient records from a remote server. The digital assistant may be configured to transform the at least a portion of the unstructured data to structured data based on a validation requirement for the at least one data field. The validation requirement may correspond to one or more of a National Emergency Medical Service Information System (NEMSIS) standard or an HL7 Fast Healthcare Interoperability Resources (FHIR) standard. The validation requirement may include a rule for one or more required fields in the EMS record, and the digital assistant may be configured to confirm that the one or more required fields include data values, identify unfilled required fields, and prompt the user to provide the unstructured data for the unfilled required fields.

In the system, the digital assistant may be configured to identify at least one second data field as being procedurally related to the at least one first data field based on a predictive workflow, generate at least one prompt that requests at least one second value of at least one second data field in the EMS record based on the at least one first data field, and present the at least one prompt to the user via the one or more user interface devices. The predictive workflow may identify procedurally related fields based on one or more of a geolocation, an EMS transport mode, a type of EMS service, one or more medical provider preferences, one or more medical protocols, one or more medical procedures, one or more medical assessments, one or more environmental attributes, presence of one or more medical diagnostic devices, one or more patient historical medical conditions, one or more patient demographic attributes, one or more crew capabilities or certifications, one or more patient current medications, and one or more patient allergies. The EMS transport mode may include a medivac service or an ambulance service. The type of EMS service may include a scheduled call or an emergency call. The type of EMS service may include a medical emergency identification from a dispatch service. The predictive workflow may be customizable by an EMS organization.

In the system, the digital assistant may be further configured to operate in two or more of a plurality of interactivity modes; and switch from a first interactivity mode to a second interactivity mode based on additional unstructured data captured by the user interface device. The plurality of interactivity modes may include two or more of a user-driven mode in which the digital assistant is configured to follow express commands of the user; a predictive mode in which the digital assistant is configured to autonomously navigate to one or more sections of the EMS record procedurally related to a data field of the plurality of data fields referenced in the additional unstructured data; a confirmation mode in which the digital assistant is configured to prompt the user to confirm values of data fields referenced in the additional unstructured data prior to population of the data fields with the values; an observational mode in which the digital assistant is configured not to prompt the user to confirm the values of the data fields referenced in the additional unstructured data prior to population of the data fields with the values; and a conversational mode in which the digital assistant is configured to prompt the user for additional values of additional data fields procedurally related to a data field of the plurality of data fields referenced in the additional unstructured data.

In the system, the express commands may include one or more of a command to navigate to a specific UI control within the user interface or a command to store values in specific data fields of the EMS record. The one or more user interface devices may include one or more of a scanner, a keyboard, a touch screen, a microphone, a virtual reality device, and a speaker. The one or more user interface devices may include a camera and the digital assistant may be configured to process a camera image to generate structured text from one or more of a medication label, handwritten text, an ECG tape and/or a screen shot of a medical device display, a driver's license, an insurance card, a payer explanation of benefits, and a hospital or billing company statement. The memory and the at least one processor may be disposed in a mobile computing device. The mobile computing device may include a smartphone.

In the system, at least a portion of the one or more user interface devices may be disposed in the mobile computing device. To identify the at least one first ePCR data field corresponding to the unstructured data may include to identify, using at least one natural language processor, at least one intent expressed within the unstructured data to document at least one value of at least one data element defined in an ePCR standard; and extract at least one slot value from the unstructured data that specifies an identifier of the at least one first data field. The at least one natural language processor may be trained using textual structures used by the users of the EMS record. The users may include one or more of EMS caregivers, hospital caregivers, hospital administrators, EMS dispatch operators, billing personnel, payer personnel, and third-party collection agencies. The textual structures used by the users may include individual sentences that include at one or more slot values that specify identifiers of data elements required by the EMS record and one or more slot values that specify values for the data elements. The textual structures may be constructed using data elements defined in a data standard for the EMS record and valid values of the data elements.

In the system, the textual structures may be specific to one or more of a period of time, a location of the users, and a type of EMS medical services. The at least one natural language processor may include a plurality of natural language processors trained using a plurality of training data sets. The plurality of training data sets may include a context data set and a section data set for each section in the EMS record. The digital assistant may be provided at a mobile computing device and the intent may include an intent to control operation of a mobile computing device. The intent to control operation of the mobile computing device may include one or more of an intent to navigate to an identified user interface control or an intent to select a default interactivity mode of a digital assistant. The intent may include an intent to send a communication to a device distinct from the mobile computing device. To identify the intent may include to generate a metric that indicates a confidence that the intent is an actual intent, and the at least one processor may be configured to switch a default interactivity mode of the digital assistant to a confirmation mode in response to the metric being less than a threshold value and to switch the default interactivity mode of the digital assistant to an observational mode in response to the metric being greater than a threshold value.

In the system, the memory and the at least one processor may be disposed in a mobile computing device and the at least one natural language processor may be hosted locally within the mobile computing device. The at least one natural language processor may include one or more natural language processors trained using data sourced from one or more of an ePCR standard, historical ePCR records, publicly available historical NEMSIS records, historical dispatch records, historical billing account records, and historical billing claims.

In another example, a method of automatically capturing electronic patient care record (ePCR) data from a caregiver is provided. The method includes acquiring speech regarding a patient encounter, converting the speech to text, identifying at least one first value of at least one first data field of the plurality of data fields based on the text, populating the at least one first data field with the at least one first value, generating at least one prompt that requests at least one second value of at least one second data field of the plurality of data fields based on the at least one first data field, and presenting the at least one prompt to the caregiver via at least one output device.

Examples of the method can include one or more of the following features.

The method may further include identifying the at least one second data field based on an organizational structure of the ePCR. The method may further include identifying the at least one second data field as being procedurally related to the at least one first data field and generating the at least one prompt in response to the identification of the procedural relationship. The method may further include rendering the one or more prompts via one or more of a speaker or a touchscreen. The method may further include acquiring camera images and processing the camera images to record one or more of an identifier of medication from a medication label, handwritten text, electrocardiogram (ECG) information from an ECG tape and/or a screen shot of a medical device display, driver's license information, insurance card information, or patient information from a face sheet. The method may further include identifying, based on the text, a first physiologic sensor that generated the at least one first value; converting additional speech to additional text; identifying at least one third value of the at least one first data field based on the additional text; identifying, based on the additional text, a second physiologic sensor that generated the at least one third value; identifying the second physiologic sensor as being a sensor of record for the at least one first data field based on a clinically derived sensor preference; and replacing the at least one first value in the at least one first data field with the at least one third value. The method may further include operating in two or more of a plurality of interactivity modes; and switching from a first interactivity mode to a second interactivity mode based on additional speech.

In the method, the plurality of interactivity modes may include two or more of a user-driven mode; a predictive mode; a confirmation mode; an observational mode; and a conversational mode. The method may further include locally executing a natural language processor configured to convert unstructured text to structured text. In the method, the natural language processor may be trained to identify, within communications articulated in a human language, data elements defined in an ePCR standard. In the method, identifying the at least one first value of the at least one first data field may include identifying, via the natural language processor, an intent in the text to document a value of a data element defined in the ePCR standard, extracting, via the natural language processor, a first slot value from the text that specifies an identifier of the data element, extracting, via the natural language processor, a second slot value from the text that specifies a value of the data element, and mapping the identifier of the data element to an identifier of the at least one first data field, and populating the at least one first data field may include to convert the value of the data element to the at least one value. The method may further include determining whether the value of the data element is valid according to the ePCR standard. The method may further include identifying the at least one second data field as being procedurally related to the at least one first data field based on a predictive workflow.

In another example, a method of natural language processing is provided. The method includes receiving unstructured information expressed in human language; identifying, using at least one natural language processor, an intent expressed within the unstructured information to document at least one value of at least one data element defined in an ePCR standard; and storing, in the memory and responsive to identification of the intent, the at least one value in association with an identifier of the at least one data element.

Examples of the method may include one or more of the following features.

The method may further include receiving the unstructured information as an audible utterance, rendering the audible utterance as text using an automated speech recognition (ASR) engine, and identifying the intent expressed within the text. The method may further include receiving the unstructured information as typed text input and identify the intent expressed within the text. In the method, the ePCR standard may be one or more of a National Emergency Medical Service Information System (NEMSIS) standard or a Fast Healthcare Interoperability Resources (FHIR) standard. In the method, storing the at least one value includes extracting, via the at least one natural language processor, a first slot value from the text that specifies an identifier of the data element; and extracting, via the at least one natural language processor, a second slot value from the text that specifies a value of the data element. The method may further include determining whether the value of the data element is valid according to the ePCR standard. The method may further include mapping the identifier of the data element to a data field of a plurality of fields in an ePCR; and populating the data field with the value of the data element. The method may further include transforming the value of the data element to generate a transformed value, wherein to populate the data field includes to populate the data field with the transformed value. The method may further include training, the at least one natural language processor using textual structures used by caregivers including EMS personnel.

In the method, the textual structures used by the caregivers may include individual sentences that include slot values that specify identifiers of data elements defined in the ePCR standard and slot values that specify values for the data elements. The method may further include constructing the textual structures using the data elements defined in the ePCR standard and valid values of the data elements. In the method, the textual structures may be specific to one or more of a period of time, a location of caregivers, and a type of medical service conducted by the caregivers. In the method, the type of medical service may include emergency medical care in a mobile environment, medical care in a mobile environment, or non-emergency medical transport. In the method, the at least one natural language processor may include a plurality of natural language processors trained using a plurality of training data sets. In the method, the plurality of training data sets may include a context data set and a section data set for each section in the ePCR standard. In the method, the intent may include an intent to document one or more of patient data, a task undertaken by a caregiver, or information required by a section of the ePCR. In the method, the intent may include an intent to control operation of the mobile computing device.

In the method, the intent to control operation of the mobile computing device may include one or more of an intent to navigate to an identified user interface control or an intent to select a default interactivity mode of a digital assistant. In the method, the intent may include an intent to send a communication to a device distinct from the mobile computing device. In the method, identifying the intent may include generating a metric that indicates a confidence that the intent is an actual intent. The method may further include switching a default interactivity mode of a digital assistant to confirmation mode in response to the metric being less than a threshold value. The method may further include switching a default interactivity mode of a digital assistant to observational mode in response to the metric being greater than a threshold value. The method may further include identifying, based on at least one value of the at least one data element, a first source device that generated the at least one value; receiving, additional unstructured information expressed in the human language; identifying at least one additional value of the at least one data element based on the additional unstructured information; identifying, based on the additional unstructured information, a second source device that generated the at least one additional value; identifying the second source device as being a device of record for the at least one data element; and storing the at least one additional value in association with the identifier of the at least one data element. In the method, the at least one natural language processor may be hosted locally within the mobile computing device. The mobile computing device may include an smartphone and/or an edge server communicably coupled with the smartphone via a local area network. In the method, the at least one natural language processor may include one or more natural language processors trained using data sourced from one or more of an ePCR standard, a medical device, shorthand terminology, or a customer specific vocabulary.

In another example, a patient data charting device for automatically capturing electronic patient care record (ePCR) data from a caregiver is provided. The device includes a memory storing an ePCR comprising a plurality of data fields, the plurality of data fields comprising at least one first ePCR data field; at least one user interface device configured to receive input comprising unstructured data corresponding to a human language communication regarding a patient encounter; and at least one processor configured to execute operations to identify at least one first ePCR data field corresponding to the unstructured data, transform at least a portion of the unstructured data to structured data comprising at least one data field value based on a validation requirement for the at least one first data field, and populate the at least one first data field in the ePCR with the structured data.

The patient data charting device can include one or more of the following features.

In the patient data charting device, the at least one user interface device may include a microphone and the at least one processor may be configured to transform the unstructured data based on a speech-to-text conversion from at least a first portion of the input received via the microphone. The at least one user interface device may include one or more of a touchscreen, a scanner, a camera, a keyboard, and a virtual reality device. The validation requirement may include at least one of a data field format requirement and a data field rule. To identify the at least one first ePCR data field corresponding to the unstructured data may include to identify, using at least one natural language processor, at least one intent expressed within the unstructured data to document at least one value of at least one data element defined in an ePCR standard; and extract at least one slot value from the unstructured data that specifies an identifier of the at least one first data field. The at least one user interface device may further include a speaker and the at least one processor may be configured to identify at least one second ePCR data field as being procedurally related to the at least one first ePCR data field based on a predictive workflow for the ePCR, generate at least one prompt associated with the at least one second ePCR data field, and present the at least one prompt to the caregiver via the speaker and the touchscreen. The at least one processor may be configured to identify a context corresponding to one or more of emergency medical services interventions and procedures for natural language processing based on the unstructured data, and select the predictive workflow based on the identified context. The predictive workflow may provide an order of population for fields of the ePCR based on one or more of a medical care protocol, historic medical outcomes, and an observed order of population for fields of the ePCR. The predictive workflow may be customizable by an EMS organization. The at least one prompt may include a request for input corresponding to at least one second value for the at least one second ePCR data field. The at least one prompt may include one or more of an instruction, a reminder, and an alarm corresponding to an intervention associated with the at least one second ePCR data field. The at least one prompt may include a request for confirmation of the at least one data field value prior to population of the at least one first ePCR data field. The at least one first ePCR data field and the at least one second ePCR data field may correspond to different sections of the ePCR.

The patient data charting device may further include a camera configured to acquire images. In the patient data charting device, the at least one processor may be configured to process the images to record one or more of an identifier of medication from a medication label, electrocardiogram (ECG) information from an ECG tape and/or a screen shot of a medical device display, driver's license information, insurance information, or patient information from a face sheet.

The patient data charting device may further include a camera configured to acquire images of handwritten text. The at least one processor may be configured to process the images to generate the unstructured data from the handwritten text. The images of handwritten text comprise images of handwritten text on a medical glove. In the patient data charting device, the at least one processor may be configured to identify a context of the handwritten text, identify at least one element of the handwritten text that is inconsistent with the context, and replace the at least one element of the handwritten text with a new element that is consistent with the context.

In the patient data charting device, the at least one processor may be configured to transform the at least a portion of the unstructured data to structured text using a locally executed natural language processor configured to convert unstructured text to structured text, the natural language processor being trained to identify, within communications articulated in a human language, data elements defined in an ePCR standard. The ePCR standard may be one or more of a National Emergency Medical Service Information System (NEMSIS) standard or a Fast Healthcare Interoperability Resources (FHIR) standard. The at least one processor may be further configured to validate the at least one data field value.

The patient data charting device may further include one or more of a smartphone, a tablet, a portable computing device, a wearable computing device, or combinations thereof. In the patient data charting device, the caregiver may include one or more of an emergency medical technician, a paramedic, a medic, a physician, a nurse, and a medical scribe. The at least one processor may be configured to transform the at least a portion of the unstructured data to structured data and populate the at least one first data field in the ePCR via interoperations with one or more processors of a server computer distinct from the patient data charting device. The server computer may be either a cloud server or an edge server based on availability of a network connection to the cloud server. The interoperations may include at least one request for the one or more processors to execute natural language processing.

The patient data charting device may include an edge server configured to communicatively couple to a cloud server and the at least one user interface device. The edge server may be disposed at an emergency transport vehicle or in a medical device carrying case. The edge server may be integrated into a medical device.

Other capabilities may be provided and not every implementation according to the disclosure must provide any, let alone all, of the capabilities discussed. Further, it may be possible for an effect noted above to be achieved by means other than that noted, and a noted item/technique may not necessarily yield the noted effect.

### BRIEF DESCRIPTION OF THE DRAWINGS

Various aspects of the disclosure are discussed below with reference to the accompanying figures, which are not intended to be drawn to scale. The figures are included to provide an illustration and a further understanding of various examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure. A quantity of each component in a particular figure is an example only and other quantities of each, or any, component could be used.

FIGS. 1A, 1B, and 1C are a schematic diagram illustrating an example patient encounter involving an EMS digital assistant in accordance with an example of the present disclosure.

FIGS. 2A through 2J are front views of user interface screens displayed by an EMS digital assistant in accordance with an example of the present disclosure.

FIG. 3A is a schematic diagram of a patient charting system that includes multiple EMS digital assistants in accordance with an example of the present disclosure.

FIG. 3B is a schematic diagram of a patient charting system that includes multiple EMS digital assistants in accordance with an example of the present disclosure.

FIGS. 4A through 4F are front views of user interface screens displayed by a patient charting system and an EMS digital assistant in accordance with an example of the present disclosure.

FIG. 5A is a schematic diagram illustrating an EMS digital assistant in detail and in accordance with an example of the present disclosure.

FIGS. 5B and 5C are schematic illustrations of examples of reciprocal modifications in the functioning of the EMS digital assistant and the caregiver workflow.

FIG. 6 is a flow diagram illustrating another dialog process executed by an EMS digital assistant in accordance with an example of the present disclosure.

FIG. 7A is a flow diagram illustrating a user interface navigation process executed by an EMS digital assistant in accordance with an example of the present disclosure.

FIG. 7B is a flow diagram illustrating an ePCR data recordation process executed by an EMS digital assistant in accordance with an example of the present disclosure.

FIG. 7C is a flow diagram illustrating an ePCR image capture process executed by an EMS digital assistant in accordance with an example of the present disclosure.

FIG. 7D is a flow diagram illustrating an ePCR data reporting process executed by an EMS digital assistant in accordance with an example of the present disclosure.

FIG. 8 is a flow diagram illustrating an ePCR population process executed by an EMS digital assistant in accordance with an example of the present disclosure.

FIG. 9 is a data flow diagram illustrating a training system and process in accordance with an example of the present disclosure.

FIG. 10A is a schematic block diagram illustrating an example of a logical and physical architecture of an EMS digital assistant as part of an EMS SaaS platform.

FIG. 10B is a schematic block diagram illustrating an example of a logical and physical architecture of an EMS digital assistant as part of an EMS SaaS platform.

### DETAILED DESCRIPTION

Often in an emergency encounter, an EMS caregiver interacts with a critically ill patient for the first time and with no prior medical knowledge about the patient. The emergency encounter is often in a non-medical environment like a home, office, or gym. In many cases, the encounter occurs in the chaotic environment of a fire scene, a car accident, or a mass casualty scene.

Within this challenging environment, the EMS caregiver is tasked not only with helping patients but also recording information descriptive of the encounter and the patient. Recordation of information enables a system, for example, a digital assistant system as described herein, to provide caregiver guidance. Such guidance improves the efficiency and accuracy of patient care which in turn improves the efficacy of this care. As discussed herein, caregiver tasks may be procedurally related based on the workflow of a caregiver in providing interventions (e.g., to triage a patient or prolong life until comprehensive diagnosis is available) and/or in diagnosing an etiology. These procedural relationships may be learned by a digital assistance system, for example, based on historical patterns of caregiver workflow, medical treatment protocols, differential diagnosis procedures, and context of care (e.g., geography, mode of transport, locale or municipality, presenting conditions, etc.). Based on this learning, the digital assistance system may provide caregiver guidance and predictive prompting to ensure that the caregiver provides comprehensive and accurate interventions. Further, the care process flow may improve the functioning of the digital assistance system. For example, the digital assistance system may adapt its model selection and utilization based on the care process flow to increase the efficiency and accuracy of a natural language processor and to enable implementation of the natural language processor on a limited capacity computing device, such as a smartphone without an Internet connection, or within a mobile distributed computing system made up of the limited capacity computing device and a mobile edge server, as described herein. This technical advantage is critical in practice where the scene of an emergency may lack Internet connectivity (e.g., a rural highway, a parking garage, an individual residence, etc.). Additionally, given the currently ubiquitous nature of smartphones, a caregiver may receive guidance and record information using a readily accessed and familiar device.

Some of the first activities undertaken by the EMS caregiver within a patient encounter are to observe, examine, and/or communicate with the patient to collect information relevant to the patient's medical condition. This patient information can include, for instance, patient biographical information, past medical conditions, medications, allergies, vital signs, mental state, and the like. An accurate understanding of patient information is critical for efficacious medical treatment during the encounter with the patient and during follow-on care at a medical facility. The patient information informs both impressions reached and interventions performed by the EMS caregiver during the patient encounter and diagnoses determined and treatments performed by physicians subsequent thereto.

For example, consider an illustrative scenario of a crew of EMS caregivers in an ambulance being called upon to treat a patient suffering from an emergency medical condition (e.g., cardiac arrest, trauma, respiratory distress, drug overdose, etc.) and to transport the patient to a hospital. During the course of this emergency encounter, the EMS caregivers may be required to travel to a patient's scene, determine and record patient information, such as patient symptoms observed during the encounter, patient physiological parameters (such as heart rate, ECG traces, temperature, blood-oxygen data, and the like) measured during the encounter, triage classification, and treatments or medications administered during the encounter. Other patient information recorded may include patient demographic information and billing/insurance information. In addition to patient information, the EMS caregivers may be also expected to record information regarding the encounter itself, such as the type of service requested, response mode, and the like.

To provide a complete and accurate record of each encounter that includes patient and encounter information and that provides comprehensive and adapted caregiver guidance, as described above, an EMS caregiver may complete an ePCR. ePCRs include data fields configured to store a comprehensive set of patient and encounter information according to a schema that controls the structure of the data provided to the digital record. In some examples, the schema may be a multi-agency standard that provides a compliance architecture to allow transfer of data and data interoperability between individual agency systems and enables entry of data in a centralized database. An example of such a standard is the National Emergency Medical Services Information Standard (NEMSIS) for emergency care medical record data collection. Additionally, the schema may utilize standardized data formatting that enables communication between medical record systems. For example, the HL7®FHIR® (Health Level Seven Fast Healthcare Interoperability Resources) standard defines how healthcare information can be exchanged between different computer systems such as those servicing emergency care and those servicing hospitals. Other examples of standards include, but are not limited to, an HL7 version 2, version 3 or CDA standard, an Electronic Data Interchange (EDI) Healthcare including, 270, 271, 276, 277, 278, 820, 834, 835, 837P and 8371 standard, SNOMED CT standard, diagnosis classification ICD standard, and procedure code HCPCS and CPT standards.

From a theoretical perspective, ePCRs are completed contemporaneously with, i.e., during, the ongoing encounter. However, entering this data during the encounter diverts the attention of the EMS caregiver away from the patient and reduces the amount of time the EMS caregiver can devote to patient care. This is particularly true if the documentation process relies on hands-on data entry. For example, data entry to a computing device, such as a tablet, laptop, or other mobile device processing the ePCR may require manual entry via a touchscreen, keyboard, stylus, or another manual data entry device. This aspect of ePCR screens can make it time consuming and difficult to enter patient and encounter information. In some implementations, the ePCR may include 50-1000 fields for which a data entry is required (e.g., required by laws of a state or another jurisdiction and/or required for adherence to a data collection standard). Since the user may not be able to reduce or customize the number of data entry fields, at least at the point of care, the accuracy and completeness of the ePCR may improve as a result of automated filling of at least a portion of these fields. The voluminous number of required fields may cause users to skip or rush through these fields, particularly in the context of an emergency response. However, skipped, inaccurate, and/or incomplete data entry may negatively affect patient care and patient outcomes. Such reduction or inaccuracy reduces the ability of a digitally assisted recordation system to provide caregiver guidance and results in a reduction in the accuracy and completeness of information passed from an initial emergency care encounter to a subsequent hospital encounter.

NEMSIS is just one example of an official EMS data collection standard for EMS agencies which allows transfer of data between systems and provides a national EMS repository for reporting and research. NEMSIS provides consistent definitions of data elements used in EMS and other pre-hospital care settings. The NEMSIS data collection via NEMSIS-compliant ePCRs may enable analysis of this data for evaluation of and evidence-based improvements in patient care across an array of EMS agencies. In particular, the NEMSIS-compliant ePCRs conform to a structured XML standard for the ePCR data. NEMSIS and the XML standard are examples only and other formats and/or content requirements are within the scope of this disclosure. As a practical matter, ePCRs are often only partially completed during the encounter, or require a dedicated documentarian, because the attention and focus of the EMS caregiver are properly with the patient. In fact, the combination of the length and complexity of ePCRs and the state of existing technology make their completion so onerous that EMS caregivers often resort to recordation short-cuts, such as writing notes on scrap paper, backs of gloves, ECG tape, or other readily available handwriting stock. Worse still, some EMS caregivers wait until an encounter has concluded to start and/or complete an ePCR. Post hoc completion of the ePCR increases inaccuracies and introduces delay into the overall continuity of care provided to the patient because this practice requires the caregiver to remember what transpired during the encounter and, in some instances, what portions of the ePCR have and have not been completed. While some ePCR programs include reminders to complete required fields, this feature does not guarantee that all optional fields have been properly populated to reflect the encounter.

Thus, and in accordance with at least some examples disclosed herein, an EMS digital assistant is provided. The EMS digital assistant addresses the issues articulated above, among others, through implementation of a unique combination of features. For example, in some implementations, the EMS digital assistant is a computer-implemented process that provides EMS caregivers with a voice-controlled, predictive workflow implemented on a smartphone for guiding a caregiver and completing an ePCR. Some implementations can additionally control a camera to provide scanning capabilities and a user interface to render prompts to caregivers to perform predefined activities and/or enter charting input that specifies ePCR data. In certain examples, an EMS digital assistant hosted by a smartphone and/or an edge server can transfer ePCR data to the edge server, a cloud server, a tablet, or laptop to enable EMS caregivers to complete an ePCR on the larger form factor device. In some examples, the ePCR data transferred to the cloud server is accessible by the tablet, laptop, or other large form factor device.

In certain examples, the EMS digital assistant is configured to recognize and respond to human language. In these examples, the EMS digital assistant can execute a variety of helpful operations without requiring the caregiver's attention—e.g., by detecting, recognizing, and acting on human language communications that naturally occur within a patient encounter. In addition, in situations where an EMS caregiver opts to interact with the EMS digital assistant directly, such interactions can be carried out verbally and without the need to manually navigate one or more user interface screens, thereby increasing efficiency. Thus the natural language processing features of the EMS digital assistant allow the EMS caregiver to focus on patient treatment rather than device interaction.

In some examples, the helpful operations that the EMS digital assistant is configured to execute include verbal device control; population of ePCR portions based on recognized human language; and prediction of, and follow-up regarding, workflows procedurally related to recognized human language. For instance, in certain examples, the EMS digital assistant recognizes values of ePCR data fields specified within the unstructured text that makes up human language communications and autonomously validates, transforms, and stores the recognized values within the ePCR data fields. Moreover, in some examples, the EMS digital assistant follows-up on these recognized values by prompting the EMS caregiver to perform procedurally related tasks and/or to provide procedurally related patient or encounter information. These prompts can be verbal and/or visual, depending on the user interface modality being utilized by the EMS caregiver.

Thus, in at least some implementations, the EMS digital assistant provides for efficient, intuitive, and predominantly hands-free population of at least portions of an ePCR via natural language processing. Further, in these implementations, the EMS digital assistant prompts the EMS caregiver to input ePCR data relevant to the current activities being performed by the EMS caregiver. These prompts can include, for example, prompts to scan medication, handwritten materials, and other visually communicated information as well as verbally communicated information. Additionally, in some implementations, the EMS digital assistant confirms and/or corrects possibly erroneous ePCR data during its validation and transformation processes and by following up with the EMS caregiver. These validation and transformation processes can be based on, and ensure compliance with the schema, reporting format, and/or content standard associated with the ePCR. Moreover, in some examples, the EMS digital assistant increases the efficiency of direct interactions with the EMS caregiver by navigating to particular user interface screens in response to direct commands issued by the EMS caregiver and/or by navigating to user interface screens relevant (e.g., procedurally related) to the EMS caregiver's current activities.

It should be noted that activities performed by healthcare providers are procedurally related when those activities are part of a same medical workflow. As a result of the procedural relationship between activities, the ePCR data fields corresponding to these activities are procedurally related data fields.

The workflow may correspond to a diagnostic workflow aimed at iteratively diagnosing a patient's condition and/or a treatment workflow aimed at providing interventions and treatments for a presenting and evolving patient condition. In some cases, the treatment workflow may occur without diagnosis, for example, in a triage environment where the goal is to stabilize a patient based on presenting conditions without necessarily attempting to diagnose, or succeeding at diagnosing, an etiology for those conditions. However, in some cases the treatment workflow may include iterative or differential diagnosis.

The procedural relationship of steps in the diagnostic or treatment workflow may be pre-established based on generally accepted standards of care, expressly defined policies of healthcare organizations, a medical treatment protocol, or even crew or caregiver specific modus operandi to name a few example sources. This procedural relationship may depend on a mandated protocol or order of operations and/or observed historic behavior. For example the procedural relationship may also be an expected workflow based on past observed workflows of a particular caregiver, a caregiver or EMS crew, an EMS agency, etc.

To illustrate, consider activities performed by a caregiver in providing interventions directed to a patient presenting with difficulty breathing and activities performed by the caregiver in diagnosing and treating an asthma attack. In this illustration, the group of activities performed by the EMS caregiver to address the patient's breathing difficulties are procedurally related. Likewise, the group of activities performed to diagnose and treat the patient's asthma are procedurally related. In addition, both groups of activities may be procedurally related to one another as they may address the same underlying condition.

The elements of information, e.g., the ePCR data fields, that are descriptive of, or generated by, procedurally related activities are also, themselves, procedurally related. So, for example, electrocardiogram (ECG) data collected from a patient suffering from chest pain may be procedurally related to data specifying, for example, the patient's measured heart rate, data specifying a blood oxygen level, and/or data indicating the patient's responsiveness. Additionally, the ECG data and/or a combination of the ECG data with one or more other data fields may be procedurally related evolving conditions of the patient. For example, a patient presentation of chest pain may evolve to a cardiac arrest. Such an evolution may procedurally relate the ECG data to data fields for interventions like defibrillation, administration of pharmaceuticals, ventilation procedures, and/or transport procedures.

Examples of factors that may indicate a procedural relationship between data fields and/or caregiver activities include but are not limited to: geolocation, an EMS transport mode, a type of EMS service, one or more medical provider preferences, one or more medical protocols, one or more medical procedures, one or more medical assessments, one or more environmental attributes, presence of one or more medical diagnostic devices, one or more patient historical medical conditions, one or more patient demographic attributes, one or more crew capabilities or certifications, one or more patient current medications, and one or more patient allergies

In an implementation, the data fields may be organized into data set sections that cover various aspects of the emergency encounter. These data set sections may include, for example, data sets for airway, cardiac arrest, EMS crew, medical device, dispatch, patient disposition, patient examination, patient history, injury, laboratory results, and medications. There may also be custom configurations and sections. As an example, a patient history section may include the data fields indicated below in Table 1. Examples of field values for the data fields are also provided in Table 1. The data field values may be associated with an MCD code (e.g., International Classification of Diseases code) for billing purposes.

TABLE 1

| Data field | Field value |
|---|---|
| Last name of Patient's Practitioner | Smith |
| First name of Patient's Practitioner | Chris |
| Advance Directives | none |
| Medication Allergies | Penicillin |
| Environmental/Food Allergies | Peanut |
| Medical/Surgical History | Type 2 diabetes |
| Medical History Obtained From | Patient's husband |
| Patient's Immunization | Flu |
| Immunization Year | Current year |
| Current Medications | Metformin |
| Current Medication Dose | 500 |
| Current Medication Dosage Unit | mg |
| Current Medication Administration | Oral |
| Alcohol/Drug Use | none |
| Pregnancy | yes |
| Last Oral Intake | none |

As another example of ePCR data, Table 2 below shows examples of data fields and data field values for a pre-scheduled dialysis transport.

TABLE 2

| Data field | Field value |
|---|---|
| Call Source | Phone call |
| Dispatch Center | Verifast EMS Services |
| Run Number | 47 |
| Incident Number | 56-87 |
| Dispatched Complaint | Palliative Care |
| Patient Acuity at Dispatch | Priority 4 (Non-Acute) |
| Changed Priority | Pre-Scheduled |
| Trauma call type | Medical and trauma |
| Call type | BLS |
| Response Mode | Pre-Scheduled |
| Additional Response Mode | No lights or Sirens |
| Pickup Zone | 16 |
| Response Delay | None |
| Type of Service | Interfacility transport |
| Patient Disposition | Treated & transported |

In certain examples, to recognize and respond to unstructured human language communications, the EMS digital assistant includes an NLP. The NLP can be implemented using a combination of hardware and software, such as a general or special-purpose processor (e.g. a graphics processing unit (GPU)) configured to execute a trained machine learning process. In some examples, the machine learning process is trained using data generated based on one or more data schemas which may encompass a reporting format and/or content standard for the ePCR.

As such, the NLP implemented within the EMS digital assistant is specially configured to transform unstructured text to structured data according to the schema, reporting format, and/or content standard.

In one example, the EMS digital assistant receives the following recitation, “Heart rate is 90, blood pressure is 120/80, pulse ox is 98, respiratory rate is 20.” In this example, the NLP identifies the intent of the statement as being vital signs recordation and sub-classifies the vital signs into heart rate, blood pressure, pulse ox, and respiratory rate. Next, the EMS digital assistant maps the values (90, 120/80, 98, 20) of the subclasses to corresponding ePCR data fields, transforms the values of the subclasses to the values required by the ePCR data fields, validates the transformed values, and stores the validated values in the ePCR data fields. It should be noted that transformation of the values is non-trivial in that such transformation can require more than simply changing data type. For instance, consider the blood pressure value of 120/80. This value must be parsed into systolic and diastolic components prior to validation and storage.

In another example, the EMS digital assistant receives the following recitation, “Patient reports pain as 8.” In this example, the NLP identifies the intent of the statement as being pain scale recordation. The EMS digital assistant maps the pain value (8) to a corresponding ePCR data field, transforms the pain value to the value required by the ePCR data field, validates the transformed value, and stores the validated value in the ePCR data field.

In another example, the EMS digital assistant receives and recognizes the following recitation from a patient, “I'm allergic to latex!” In this example, the NLP identifies the intent of the statement as being allergy recordation. The EMS digital assistant maps the allergy value (latex) to a corresponding ePCR data field, transforms the allergy value to the value required by the ePCR data field, validates the transformed value, and stores the validated value in the ePCR data field. It should be noted that the source of speech recognized by the EMS digital assistant can be the patient or another audio source distinct from the caregiver, in some examples.

In another example, the EMS digital assistant receives and recognizes the following recitation, “Do you take any medications regularly?Yes, I take an aspirin daily.” In this example, the NLP identifies the intent of the statement as being medication recordation. The EMS digital assistant maps the medication value (aspirin) to a corresponding ePCR data field, transforms the medication value to the value required by the ePCR data field, validates the transformed value, and stores the validated value in the ePCR data field. It should be noted that the speech recognized by the EMS digital assistant can be part of an overall conversation regarding the patient or between the patient and the caregiver, in some examples.

In another example, the EMS digital assistant receives and recognizes the following recitation, “Patient's skin is cold and clammy.” In this example, the NLP identifies the intent of the statement as being skin examination recordation. The EMS digital assistant maps the skin values (cold, clammy) to corresponding ePCR data fields, transforms the skin values to the values required by the ePCR data fields, validates the transformed values, and stores the validated values in the ePCR data fields.

In one example, the EMS digital assistant receives and recognizes the following recitation, “Intubation successful, chest rise observed.” In this example, the NLP identifies the intents of the statement as being procedure recordation and confirmation and sub-classifies the procedure into intubation and the confirmation method as chest rise. Next, the EMS digital assistant maps the values of the subclass (intubation) and method of confirmation (chest rise) to corresponding ePCR data fields, transforms the values of the subclasses and confirmation method to the values required by the ePCR data fields, validates the transformed values, and stores the validated values in the ePCR data fields.

In another example, the EMS digital assistant receives and recognizes the following recitation, “Patient refuses transport.” In this example, the NLP identifies the intent of the statement as being disposition recordation. The EMS digital assistant maps the disposition value (no transport) to a corresponding ePCR data field, transforms the disposition value to the value required by the ePCR data field, validates the transformed value, and stores the validated value in the ePCR data field.

In another example, the EMS digital assistant receives and recognizes the following recitation, “Patient is self-insured.” In this example, the NLP identifies the intent of the statement as being insurance recordation. The EMS digital assistant maps the insurance value (self-insured) to a corresponding ePCR data field, transforms the insurance value to the value required by the ePCR data field, validates the transformed value, and stores the validated value in the ePCR data field.

In some examples, the EMS digital assistant includes a machine learning process trained to identify procedural relationships between caregiver activities and ePCR data fields. In some examples, the machine learning process is trained using data generated from formal procedural guidelines and/or data generated from actual EMS calls and patient encounters. The procedural guidelines used can be standards of care that define industry-wide medical protocols and/or procedural guidelines defined by policy specific to one or more medical organizations. It should be noted that the data generated from actual EMS call and patient encounters can be retrieved from call logs and medical devices utilized within the patient encounters. As such, this data can train the machine learning process to identify procedural relationships that are both practical and specific to the organization and/or the caregiver.

In one example, the EMS digital assistant receives the following recitation, “Patient complains of chest pain.” In this example, the NLP identifies the intent of the statement as being complaint recordation and executes steps required to store the complaint value (chest pain) in a corresponding ePCR data field. In addition, the EMS digital assistant executes a procedural relationship process to identify ePCR data fields procedurally related to a chest pain protocol and prompts the caregiver to input values for the identified ePCR data fields within an order established by the chest pain protocol.

In certain examples, the EMS digital assistant implements a number of features to increase its availability to EMS caregivers. For instance, in some examples, the EMS digital assistant is configured to execute on a variety of computing devices, including personal devices routinely carried by EMS caregivers, such as smartphones. In these examples, the EMS digital assistant can accompany EMS caregivers in patient encounters with no additional cognitive load to the EMS caregiver, as EMS caregivers usually carry such devices on their person as a matter of habit. Moreover, some examples of the EMS digital assistant are configured to execute natural language processing routines locally, so that a network connection is not required for the EMS digital assistant to operate. Alternatively or additionally, in some examples, the EMS digital assistant executes within a mobile distributed system that includes an edge server that is coupled to a personal device via a local area network (LAN) connection and/or personal area network (PAN) connection. In these examples, some routines of the EMS digital assistant are executed by the personal device (e.g., within an “app”) while other routines are executed by the edge server, so that a wide area network connection is not required for the EMS digital assistant to operate. These features increase the likelihood that the EMS digital assistant will be available for use within patient encounters and helps EMS caregivers focus on patient care.

In some examples, the EMS digital assistant is configured to execute on portable devices, such as tablets or laptops, that are larger than a smartphone. For instance, in these examples, a tablet-based EMS digital assistant can transfer, into an ePCR stored locally on the tablet or an edge server, ePCR data originally gathered by a smartphone-based EMS digital assistant. The larger form factor of the tablet device or edge server hardware may be preferable to an EMS caregiver for completion of certain portions of the ePCR, such as patient disposition, final signatures, etc. In certain examples, an ePCR at least partially completed by the EMS digital assistant can be subsequently uploaded to a web-based service for post-care services and record processing.

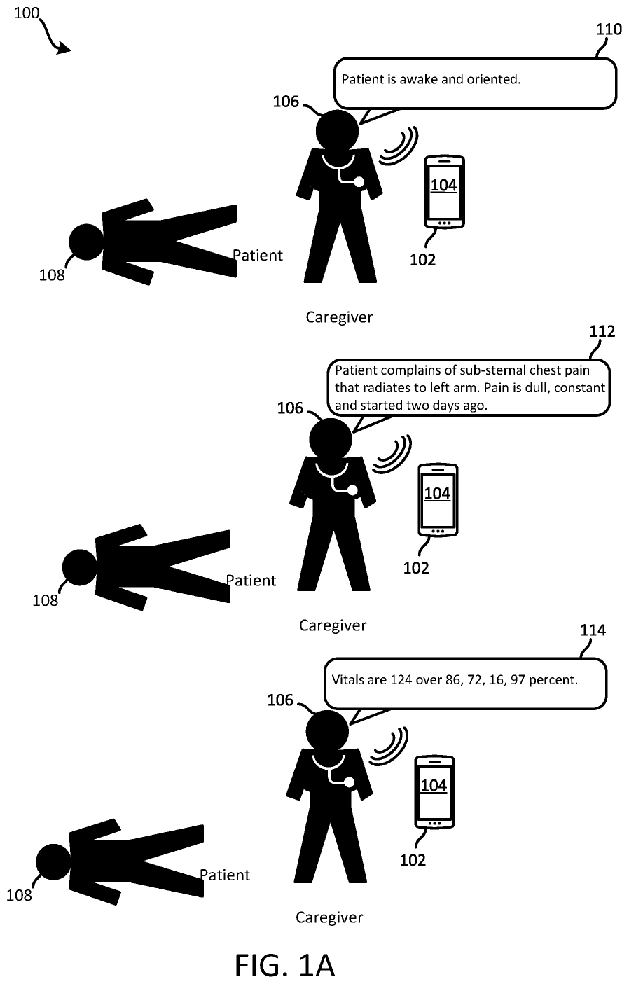

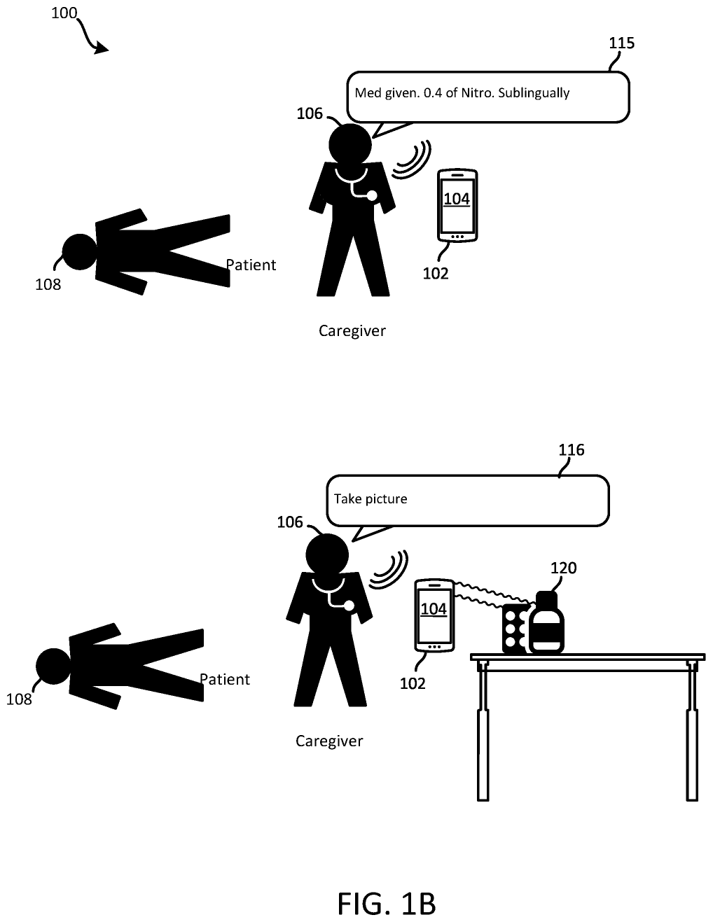

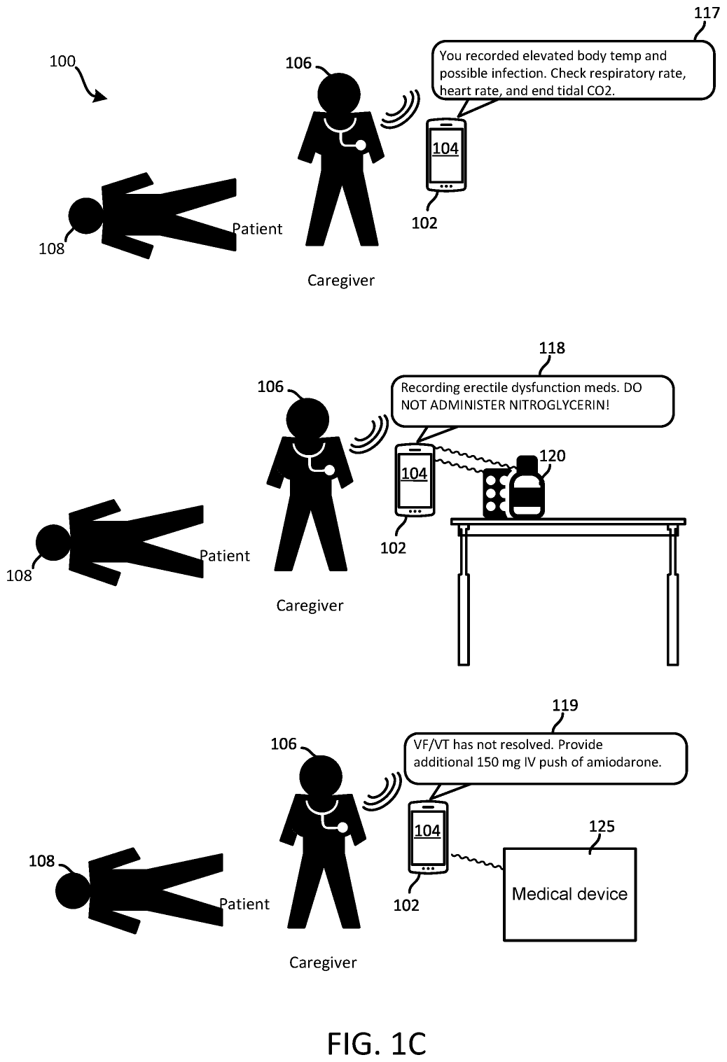

Referring to FIGS. 1A, 1B, and 1C, an example patient encounter 100 is illustrated. The encounter 100 involves a patient 108 and a caregiver 106. The caregiver carries a smartphone mobile computing device 102 (e.g., a smartphone) that includes an EMS digital assistant 104. The smartphone 102 may be, for example, a personal device of the caregiver 106 that is normally carried by the caregiver 106. The smartphone 102 may include a memory, a touchscreen, a microphone, a speaker, a network interface, and a camera. These devices may be coupled to one or more processors within the smartphone 102 that control their operation. In some examples, the one or more processors are configured to initiate and/or execute the EMS digital assistant 104. The EMS digital assistant 104 is configured to control and/or otherwise interoperate with the touchscreen, the microphone, the network interface, and the camera, as discussed further below. In certain examples, the EMS digital assistant 104 is a software application (“app”) stored in the memory, although hardware-only implementations are possible.

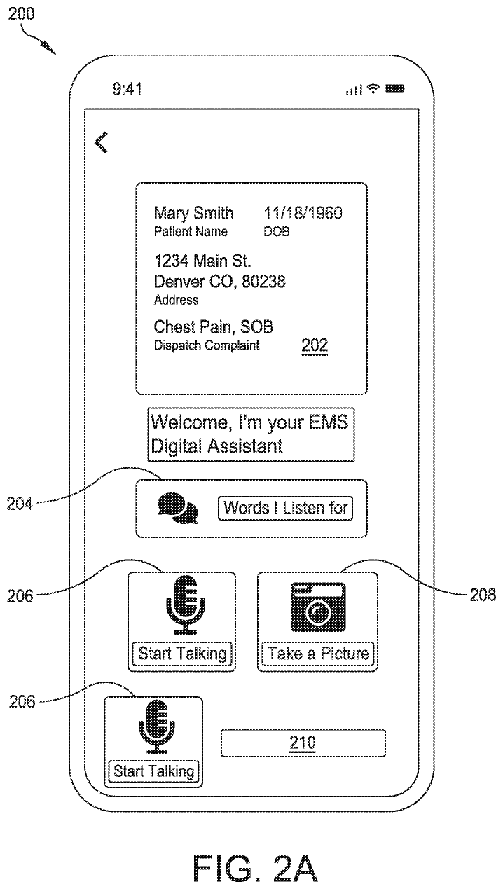

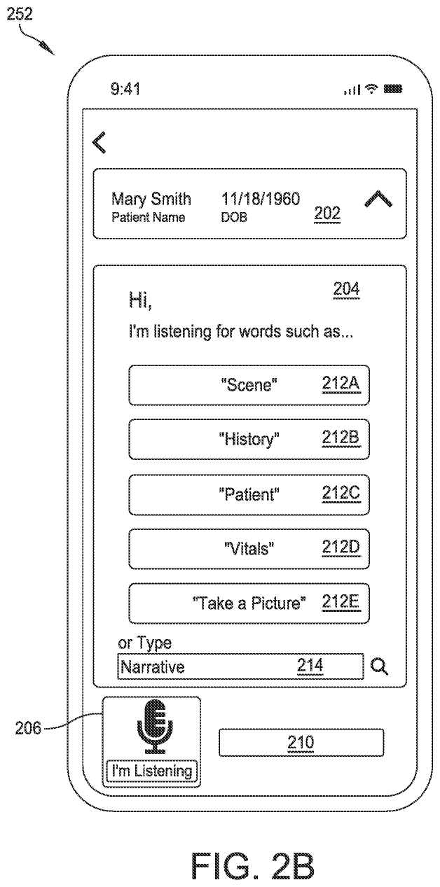

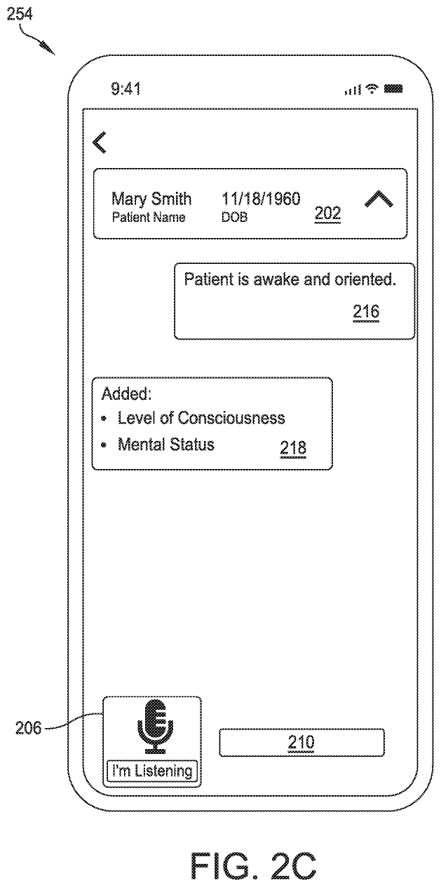

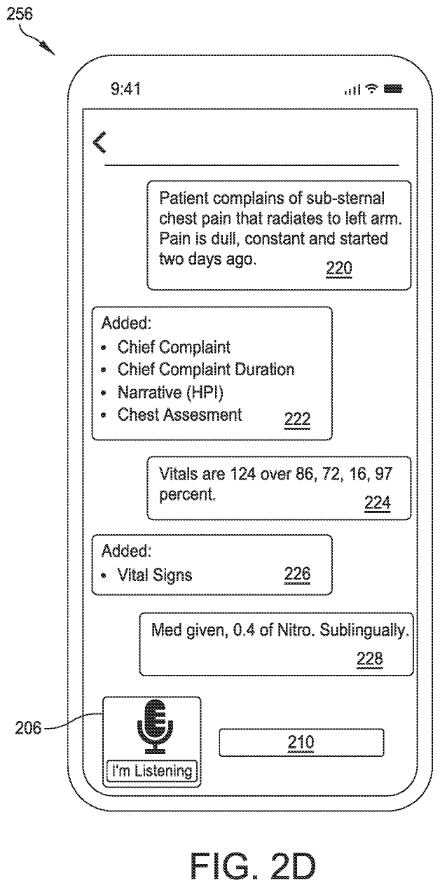

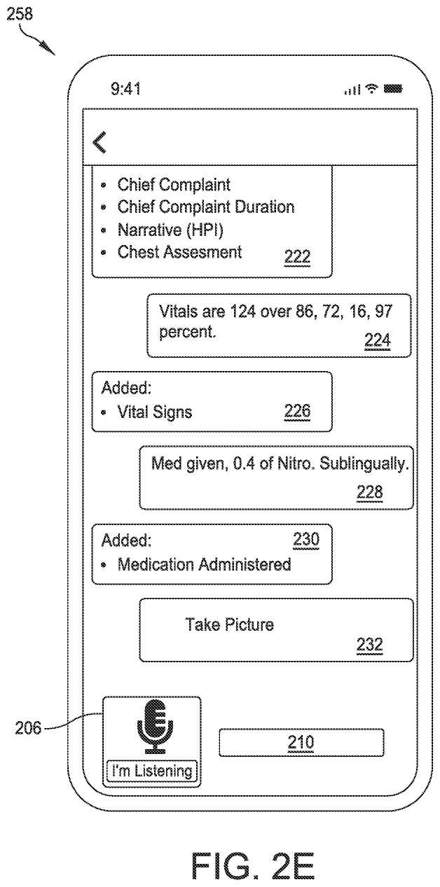

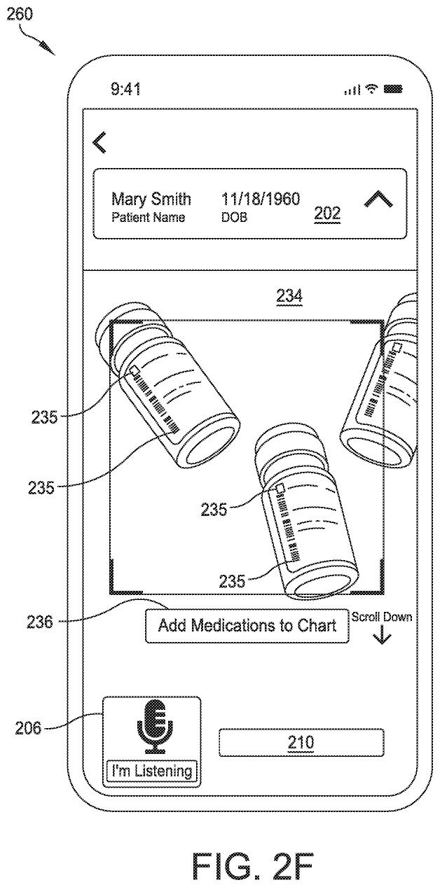

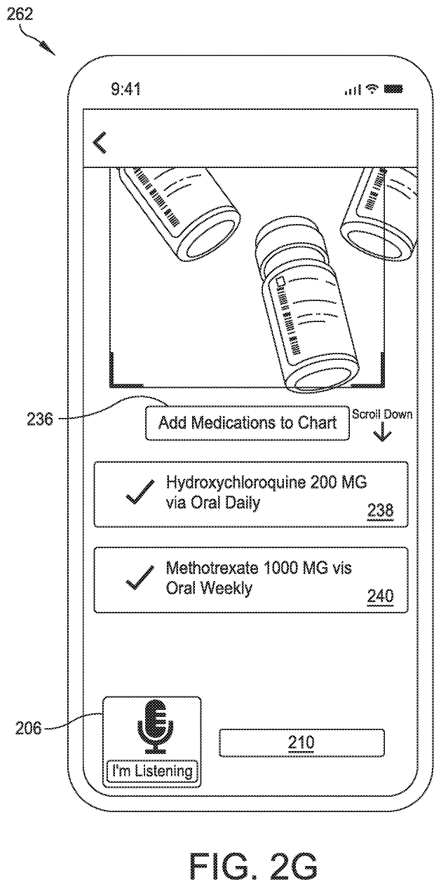

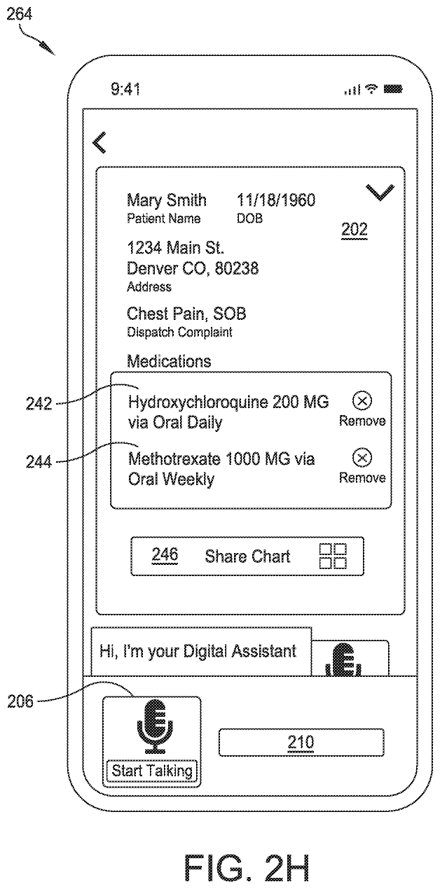

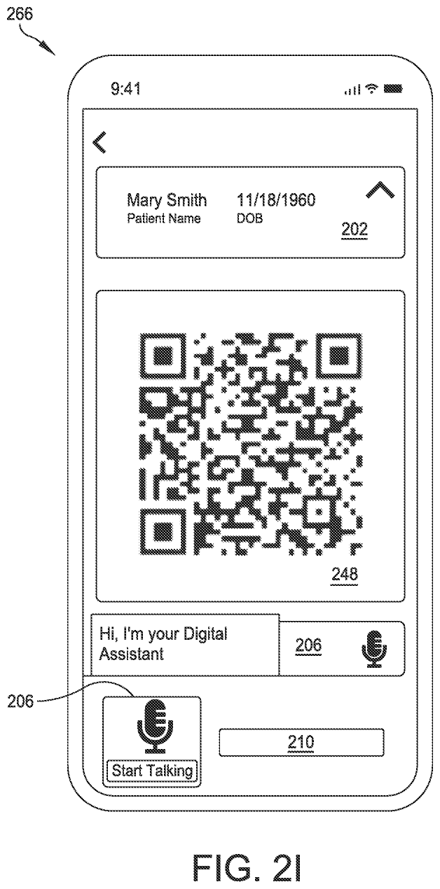

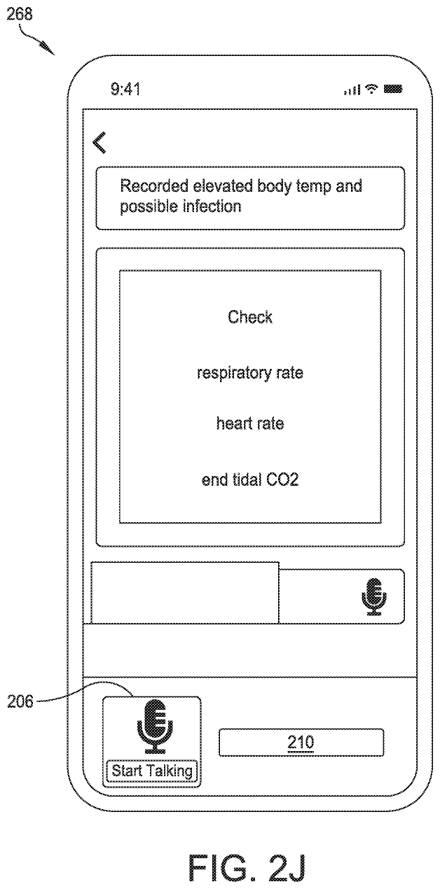

The encounter 100 will now be described with reference to FIGS. 1A-1C in combination with FIGS. 2A through 2J, which illustrate user interface screens that the EMS digital assistant 104 is configured to display during the encounter 100. In this example, prior to arriving at the scene of the encounter 100, the EMS digital assistant 104 receives dispatch information regarding the patient 108. This dispatch information includes the patient's name, date of birth, address, and complaint. The EMS digital assistant 104 is configured to display, via the touchscreen, a user interface screen 200 as shown in FIG. 2A in response to reception of the dispatch information.

As illustrated in FIG. 2A, the screen 200 includes an encounter information control 202, a recognizable words control 204, wakeup controls 206, an image capture control 208, and a text entry control 210. The EMS digital assistant 104 is configured to display the received dispatch information via the encounter control 202. Further in this example, the EMS digital assistant 104 is configured to receive tactile input via any of the controls 204-210. For instance, in one example, the EMS digital assistant 104 is configured to receive tactile input via the words control 204 and, in response thereto, expand the words control 204 to list examples of spoken words recognizable by the EMS digital assistant 104. An example of the words control 204 in an expanded state is illustrated in screen 252 of FIG. 2B. As shown in FIG. 2B, the words control 204 includes text controls 212A-212E, each of which is configured to display a recognizable word. The words control 204 also includes a word search control 214 that is configured to receive text input. The EMS digital assistant 104 is configured to search, in response to reception of such text input, a list of words recognizable by the EMS digital assistant 104 for words that match the text input. The EMS digital assistant 104 is further configured to display via the search control 214 a recognizable word that best matches the text input.

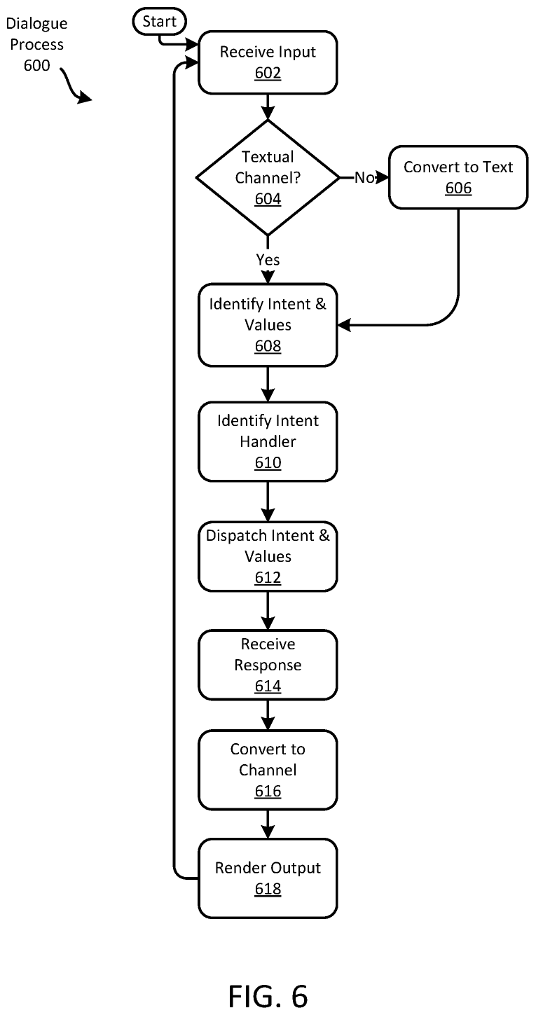

Returning to the screen 200 of FIG. 2A, the EMS digital assistant 104 is configured to initiate, in response to reception of tactile input via either of the wakeup controls 206, dialogue processing of audio data generated by the microphone and associated digitization circuitry. One example of such dialogue processing is described further below with reference to FIG. 6. Thus, each wakeup control 206 serves a purpose similar to that of a wakeup word processed by some examples of the EMS digital assistant 104 as described further below.

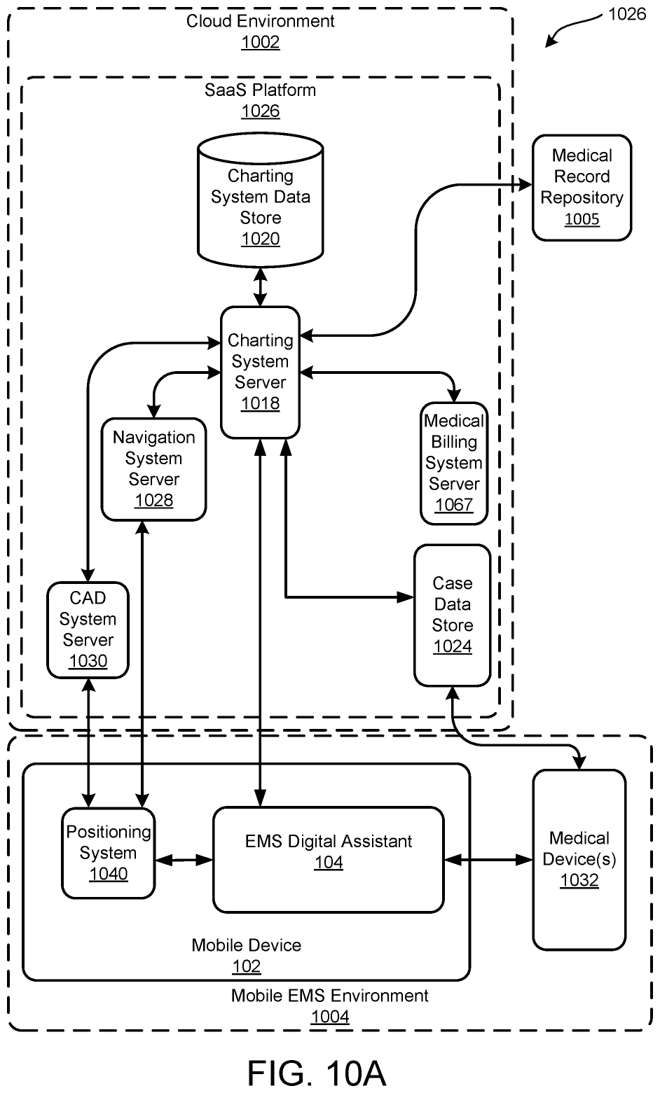

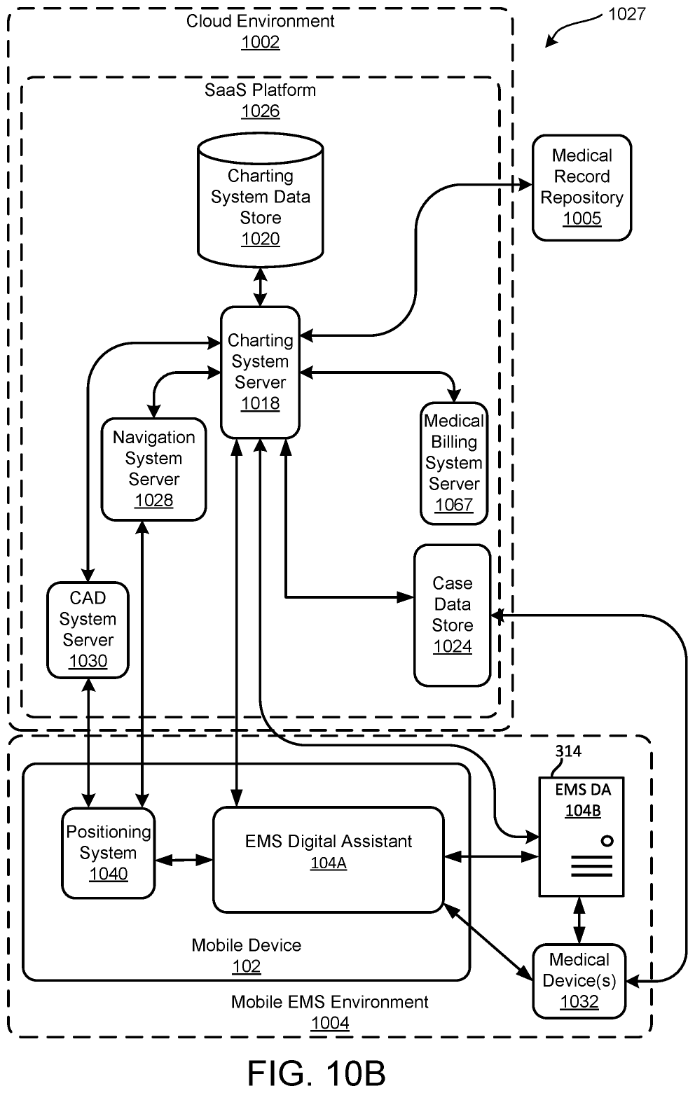

Continuing with the screen 200 of FIG. 2A, the EMS digital assistant 104 is configured to capture, in response to reception of tactile input via the capture control 208, images generated by the camera and associated digitization circuitry. In certain examples, the EMS digital assistant 104 is further configured to scan the images for symbols that encode information relevant to one or more ePCR data fields, such as barcodes, quick response (QR) codes, and typed or handwritten text. By processing these symbols, the EMS digital assistant 104 can identify information such as medications, driver's license information, insurance information, patient identifiers, hospital face sheets, physiologic parameters of the patient, and the like. As other examples, the digital assistant is configured to process a camera image to generate structured text from one or more of a medication label, handwritten text, an ECG tape and/or a screen shot of a medical device display, a driver's license, an insurance card, a payer explanation of benefits, and a hospital or billing company statement. In an implementation, the digital assistant 104 may receive one or more of the images from other entities in an EMS SaaS platform (e.g., the platforms 1026 and 1027 as shown in FIGS. 10A and 10B).

Continuing with the screen 200 of FIG. 2A, the EMS digital assistant 104 is configured to receive text input via the text input control 210. Further, the EMS digital assistant 104 is configured to initiate, in response to reception of such text input, dialog processing of text generated by the text input. One example of such dialog processing is described further below with reference to FIG. 6.

Returning to the encounter 100 of FIG. 1A, the caregiver 106 examines and/or interacts with the patient 108 and verbally notes 110 that the “Patient is awake and oriented.” In this example, the EMS digital assistant 104 is configured to receive audio data specifying this verbalization via the microphone and to execute an automatic speech recognition (ASR) process to generate a textual rendering of the verbalization. In some examples, the EMS digital assistant 104 is configured to confirm the textual rendering by displaying the textual rendering within a text control, such as text control 216 as illustrated in screen 254 of FIG. 2C. The EMS digital assistant 104 is also configured to execute a NLP that recognizes, within the textual rendering, intents to record level of consciousness and mental status of the patient. The EMS digital assistant 104 is further configured to record, based on the recognized intents, ePCR data that specifies the level of consciousness and mental status of the patient. In some examples, the EMS digital assistant 104 is configured to confirm that this recordation operation is correct by displaying an indication of the recordation operation within a text control, such as text control 218 illustrated in the screen 254.

Returning to the encounter 100 of FIG. 1A, the caregiver 106 interacts with the patient 108 and verbally notes 112 that the “Patient complains of sub-sternal chest pain that radiates to left arm. Pain is dull, constant and started two days ago.” In this example, the EMS digital assistant 104 is configured to receive audio data specifying this verbalization via the microphone and to execute the ASR process to generate a textual rendering of the verbalization. In some examples, the EMS digital assistant 104 is configured to confirm the textual rendering by displaying the textual rendering within a text control, such as text control 220 illustrated in screen 256 of FIG. 2D. The EMS digital assistant 104 is also configured to execute a NLP that recognizes, within the textual rendering, intents to record chief complaint, chief complaint duration, narrative information, and chest assessment of the patient. The EMS digital assistant 104 is further configured to record, based on the recognized intents, ePCR data specifying the chief complaint, chief complaint duration, narrative information, and chest assessment of the patient. In some examples, the EMS digital assistant 104 is configured to confirm that this recordation operation is correct by displaying an indication of the recordation operation within a text control, such as text control 222 illustrated in the screen 256.

Returning to the encounter 100 of FIG. 1A, the caregiver 106 examines the patient 108. This examination includes the use of one or more medical devices configured to detect physiologic parameters of the patient 108. The caregiver 106 verbally notes 114 that “vitals are 124 over 86, 72, 16, 97 percent.” In this example, the EMS digital assistant 104 is configured to receive audio data specifying this verbalization via the microphone and to execute the ASR process to generate a textual rendering of the verbalization. In some examples, the EMS digital assistant 104 is configured to confirm the textual rendering by displaying the textual rendering within a text control, such as text control 224 illustrated in FIG. 2D. The EMS digital assistant 104 is also configured to execute a NLP that recognizes, within the textual rendering, an intent to record vital signs of the patient. The EMS digital assistant 104 is further configured to record, based on the recognized intent, ePCR data that specifies the vital signs of the patient. In some examples, the EMS digital assistant 104 is configured to confirm that this recordation operation is correct by displaying an indication of the recordation operation within a text control, such as text control 226 illustrated in FIG. 2D. The vital signs example described above illustrates an abbreviated language feature implemented by the EMS digital assistant 104 in some examples. In these examples, the EMS digital assistant 104 is configured to execute an NLP specially trained to recognize medical terminology, syntax, and grammar utilized by caregivers. Incorporation of this specialized NLP enables the EMS digital assistant 104 to communicate with the caregiver 106 more efficiently than through the use of formal human language. For instance, in this particular example, the NLP is trained to recognize “vitals are 124 over 86, 72, 16, 97 percent” means that the patient's systolic blood pressure is 124 mmHg, the patient's diastolic blood pressure is 86 mmHg, the patient's heart rate is 72 beats per minute, the patient's respiratory rate is 16 beats per minute, and the patient's pulse oxygen is 97 percent. In various implementations, the NLP may be trained on language and textual structures of one or more of EMS caregivers, hospital caregivers, hospital administrators, EMS dispatch operators, billing personnel, payer personnel, and third-party collection agencies. In some implementations, entities across the healthcare spectrum may provide unstructured text to the EMS digital assistant, for example, via a platform such as the platforms 1026 and 1027 as shown in FIGS. 10A and 10B.

Turning to FIG. 1B, the caregiver 106 administers nitroglycerin to the patient 108 in accordance with a chest pain protocol and verbally notes 115, “Med given. 0.4 of Nitro. Sublingually.” In this example, the EMS digital assistant 104 is configured to receive audio data specifying this verbalization via the microphone and to execute the ASR process to generate a textual rendering of the verbalization. In some examples, the EMS digital assistant 104 is configured to confirm the textual rendering by displaying the textual rendering within a text control, such as text control 228 illustrated in screen 258 of FIG. 2E. The EMS digital assistant 104 is also configured to execute a NLP that recognizes, within the textual rendering, an intent to record administration of medication to the patient. The EMS digital assistant 104 is further configured to record, based on the recognized intent, ePCR data that specifies information regarding the medication administered to the patient. In some examples, the EMS digital assistant 104 is configured to confirm that this recordation operation is correct by displaying an indication of the recordation operation within a text control, such as text control 230 illustrated in the screen 258.

It should be noted that, in some examples of the recordation operations described above, the EMS digital assistant 104 is configured to transform and validate data prior to storing the ePCR data. Such transformation may include changes to data type and format (e.g., from a string to a numeric value) as well as translations to different symbols (e.g., from the word “now” to a timestamp reflecting the current time). In some cases, the transformation and validation operations are performed to ensure that the data stored in ePCR data fields meets the requirements of the schema, reporting format, and/or content standard associated with the ePCR. Also, in some examples, the EMS digital assistant 104 may prompt the caregiver 106 for additional information that is procedurally related to the populated ePCR data fields, depending on the current mode of operation of the EMS digital assistant 104, as will be described further below.

Returning to the encounter 100 of FIG. 1B, the caregiver 106 discovers medication 120 prescribed to the patient 108 and verbally commands 116 the EMS digital assistant to “take a picture” of the medication 120. In this example, the EMS digital assistant 104 is configured to receive audio data specifying this verbalization via the microphone and to execute the ASR process to generate a textual rendering of the verbalization. In some examples, the EMS digital assistant 104 is configured to confirm the textual rendering by displaying the textual rendering within a text control, such as text control 232 illustrated in FIG. 2E. The EMS digital assistant 104 is also configured to execute a NLP that recognizes, within the textual rendering, an intent to capture an image using the camera. The EMS digital assistant 104 is further configured to display, based on the recognized intent, a viewfinder control, such as the viewfinder control 234 illustrated in screen 260 of FIG. 2F. The EMS digital assistant 104 is additionally configured to interoperate with the camera to capture images, to scan the images to find symbols encoding information relevant to ePCR data fields, and to display the images within the viewfinder control 234. For instance, in some examples, the EMS digital assistant 104 is configured to scan the images for National Drug Code barcodes to find medications shown within the images. Additionally or alternatively, in certain examples, the EMS digital assistant 104 is configured to scan for other symbols, such as typed or handwritten text, that encode information relevant to ePCR data fields. In some examples, the EMS digital assistant 104 is configured to highlight symbols found within the image. For instance, as shown in the screen 260, the EMS digital assistant 104 is configured to overlay the image with one or more indicators 235 of the symbols. These indicators 235 provide the caregiver 106 with confirmation that symbols encoding information relevant to ePCR data fields were found.

In the examples of FIG. 1C, prompts from the EMS digital assistant 104 are provided to the caregiver 106. For example, the EMS digital assistant 104 may provide a prompt 117 for procedurally related activities. After recording an elevated body temperature and a possible infection in the ePCR based on information received from the caregiver 106, the EMS digital assistant may predict that the next steps in care should be to check respiratory rate, heart rate, and end tidal CO2 and prompt the caregiver 106 to perform these steps. As another example, in response to automatically recording a medical bar code indicating that patient 108 takes erectile dysfunction medications, the EMS digital assistant 104 may provide a warning that, despite a previously recordation by the caregiver 106 of a cardiac condition, the caregiver 106 should not administer nitroglycerin because of a contraindication with the erectile dysfunction medication. As a further example, the EMS digital assistant 104 may receive medical data from a medical device 125. Based on that medical data, the EMS digital assistant may automatically provide a medication alarm or reminder 119.

Turning to FIG. 2G, as illustrated in screen 262 the EMS digital assistant 104 is configured to display, in response to finding symbols encoding medication information within the image, an add medications control 236. In some examples, the EMS digital assistant 104 is configured to record, in response to reception of tactile input via the mediations control 236, ePCR data specifying identifiers of the medication information symbolized within the image. Further, in these examples, the EMS digital assistant 104 is configured to confirm successful storage of the medication information by displaying medication configuration controls 238 and 240, each of which lists the type and dosage regimen for an identified medication.

Turning to FIG. 2H, as shown in screen 264 the EMS digital assistant 104 is configured to display an expanded version of the encounter control 202 in response to input from the caregiver 106 that indicates the caregiver 106 is prepared to share the recorded ePCR data. As illustrated in screen 264, the expanded encounter control 202 includes medication controls 242 and 244 and a share chart control 246. Each of the controls 242 and 244 displays, and is associated with, medication information associated with the patient 108. The EMS digital assistant 104 is configured to receive tactile input via any of the controls 242-246. For instance, in one example, the EMS digital assistant 104 is configured to receive tactile input via the medication control 242 and, in response thereto, to delete the medication information associated with the medication control 242. In this example, the EMS digital assistant 104 is also configured to receive tactile input via the medication control 244 and, in response thereto, to delete the medication information associated with the medication control 244.

Continuing with FIG. 2H, in certain examples, as shown in the screen 164 the EMS digital assistant 104 is configured to receive tactile input via the chart control 246 and, in response thereto, to generate a unique identifier of the encounter 100, encode the identifier into a QR code, and display the QR code within a QR code control, such as the QR code control 248 illustrated in screen 266 of FIG. 2I.

Turning to FIG. 2J, in some examples, the EMS digital assistant 104 may provide visual prompts as an alternative or in addition to the verbal prompts. For example, the verbal prompt 117 from FIG. 1C is shown as a visual prompt 268.

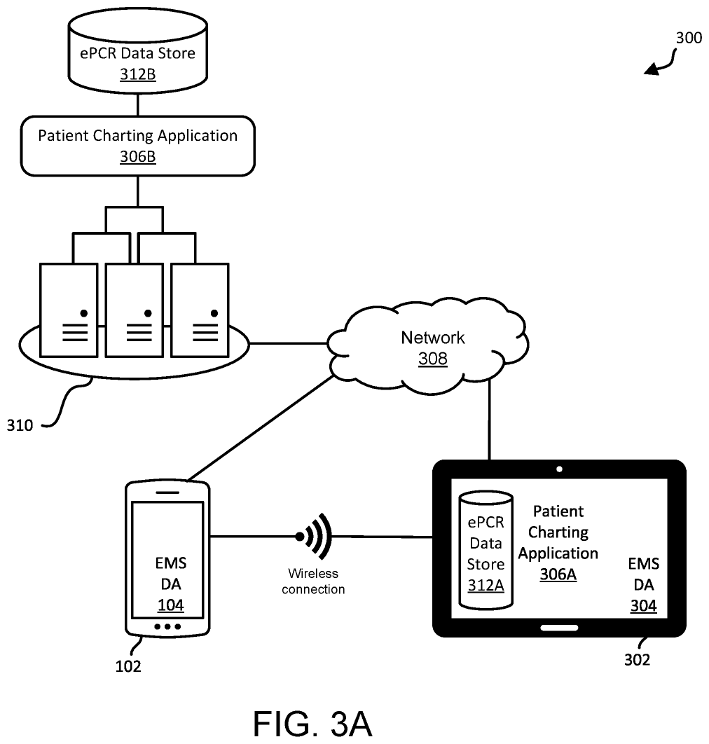

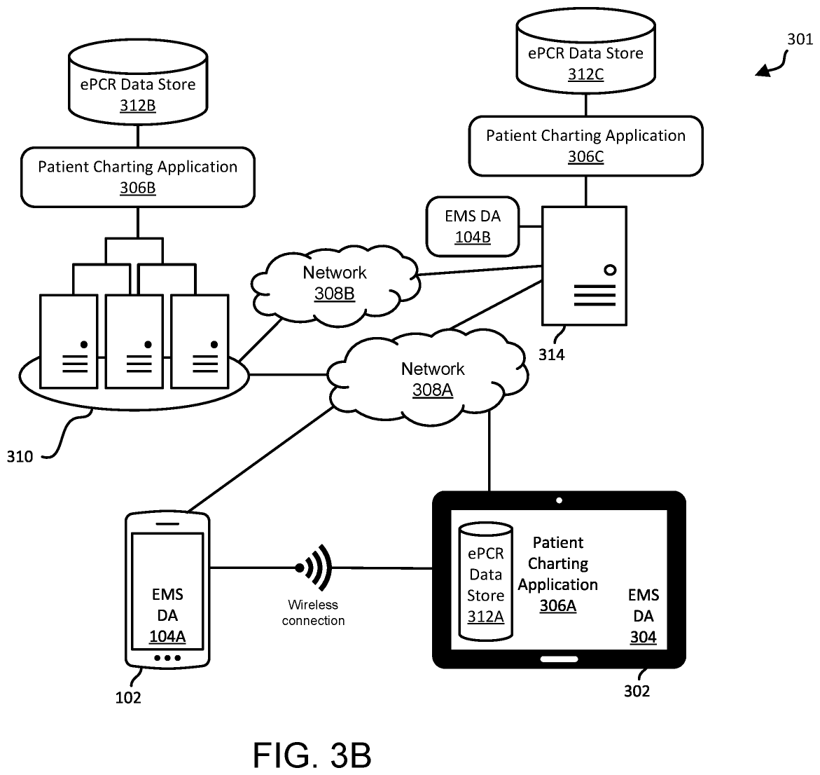

In some situations, the caregiver 106 may prefer to complete certain portions of an ePCR using a computing device that has a form factor that is larger than that of the smartphone 102. In some implementations, the EMS digital assistant 104 may be configured to transfer populated ePCR data fields to a patient charting application (e.g., emsCHARTS® patient charting application commercially available from ZOLL Medical Corporation of Chelmsford, Massachusetts in the United States) that is hosted by a computing device other than the smartphone 102. In some implementations, the EMS digital assistant 104 may incorporate the patient charting application. The EMS digital assistant 104 may be an application hosted on a portable device and capable of operation with and without a server connection (e.g., a cloud server or an edge server). With a cloud server and/or edge server connection, data recorded by the digital assistant in the ePCR at the smartphone may be accessible from other devices. For example, the digital assistant may be served to the smartphone by a charting system on the cloud server (e.g., the charting system server 1018 in FIG. 10A or 10B). The cloud server may access and store data fields populated by the digital assistant with or without a cloud server connection. Alternatively or additionally, the digital assistant may be a distributed application made up of collaborative processes hosted on the smartphone and the edge server (e.g., as illustrated and described with reference to FIG. 3B below). The smartphone and other devices, including larger form factor devices, like a laptop, tablet, server monitor connected to an edge server, etc., may access the data stored at the cloud server or the edge server. In the absence of the cloud server or edge server connection, the digital assistant at the local device (e.g., the portable device) may store the data on the local device until a cloud or edge connection is established. In an implementation, the local storage is within the application such that there is no data storage footprint once the cloud server connection or the edge server connection is established. Such an arrangement protects the privacy and security of the stored data. As part of the overall charting application, the digital assistant may be available on any device regardless of form factor, e.g., on the smartphone, the laptop, the tablet, a server monitor of the edge server, etc.

FIG. 3A illustrates one example of a system 300 that supports the implementations described herein. As shown in FIG. 3A, the system 300 includes the smartphone 102 of FIG. 1, a tablet computing device 302, a network 308, and a server environment 310. The tablet 302 hosts an EMS digital assistant 304, a patient charting application 306A, and an ePCR data store 312A. The server environment 310 hosts a patient charting application 306B and an ePCR data store 312B. The smartphone 102 may be configured to connect to the tablet 302 via a short-range wireless connection (e.g., a personal area network (PAN) connection, such as a BLUETOOTH connection, or a local area network (LAN) connection, such as a WIFI connection) and to the network 308 via a long-range wireless connection (e.g., a wide area network (WAN) connection, such as a Code-division Multiple Access (CDMA) connection or Global System for Mobile Communication (GSM) connection). Similarly, the tablet 302 may be configured to connect to the smartphone 102 via a short-range wireless connection, such as a PAN connection or LAN connection, and to the network 308 via a long-range wireless connection, such as a WAN connection. In an implementation, the tablet 302 may be pre-configured to be associated with a medical treatment, diagnostic device, and/or edge server so as to streamline wireless communication pairing without having to undergo a time-consuming inquiry and response negotiation for a secure connection to be established. The tablet 302 may be a companion device of a medical treatment and/or diagnostic device. In some implementations, the companion device is dedicated to communicating only with its corresponding medical and/or diagnostic device. In some implementations, the companion device can display sensor data in real-time from one or more physiological sensors connected to the medical treatment device. In some examples, the companion device can display a visual reproduction of the information displayed at the medical treatment device in a first display. In some examples, the visual reproduction may encompass an exact replication of the data displayed at the medical treatment device. In other examples, the visual reproduction may include data and formatting variations that can enhance viewing and comprehension of the case information by the companion device user. In some examples, display layout, magnification of each data section, physiologic waveform selection, physiologic numeric readout selection, resolution, waveform duration, waveform size, text size, font, and/or display colors may vary from what is displayed at the medical treatment device(s).

The server environment 310, which includes one or more physical and/or virtual servers, is configured to connect to the network via a robust network connection, such as a dedicated and redundant service provider connection. The network 308 is a high-availability public or private network, such as the Internet, through which computing devices exchange (transmit and/or receive) communications. The computer-implemented processes illustrated in FIG. 3A (e.g. the EMS digital assistant 104, the EMS digital assistant 304, and the patient charting applications 306A and 306B) interoperate with one another over the connections described above via one or more application programming interfaces (APIs) implemented by the processes.

It should be noted that the charting applications 306A and 306B and the data stores 312A and 312B can be configured to operate collaboratively or independently, depending on the design goals of a particular installation. For instance, in some examples, the charting application 306B serves the charting application 306A as a browser-based user interface to the tablet 302. In these examples, the charting application 306A is a thin client and relies on periodic communications with the charting application 306B to operate properly. Moreover, in these examples, the data store 312A may be maintained in browser session storage and, thus, may contain a limited amount of data that is updated periodically with data from the data store 312B. Alternatively or additionally, in some examples, the charting application 306A is an independent application configured to execute natively under an operating system of the tablet 302. In these examples, the data store 312A may contain all of the data needed for the charting application 306A to operate properly. In either case, it should be noted that the data stores 312A and 312B may exchange information periodically or in real time to maintain data currency.

In some examples, the EMS digital assistant 104 transfers recorded ePCR data to remote data stores (e.g., the data store 312A and/or the data store 312B). The EMS digital assistant 104 may be configured to execute this transfer in real time or in batches based on occurrence of one or more events (e.g., according to a time-based schedule, based on availability of sufficient network bandwidth, in response to caregiver input, etc.). This transfer may be effected by, for example, one or more API calls from the EMS digital assistant 104 to the EMS digital assistant 304, the patient charting application 306A, the patient charting application 306B, the data stores 312A and/or the data store 312B. For instance, in some examples, the EMS digital assistant 104 transfers populated ePCR data fields to the data store 312A hosted by the tablet 302. Alternatively or additionally, in some examples, the EMS digital assistant 104 transfers recorded ePCR data to the data store 312B hosted by the server environment 310. In either case, transferred ePCR data can be accessed by the EMS digital assistant 104, the EMS digital assistant 304, the patient charting application 306A and/or the patient charting application 306B. In this way, the system 300 enables the charting application 306A and/or the charting application 306B to access ePCR data fields populated by the EMS digital assistant 104. This access, in turn, enables the charting application 306A and/or the charting application 306B to interact with a caregiver (e.g., the caregiver 106 of FIG. 1) to complete or review administrative portions of an ePCR.

FIG. 3B illustrates one example of a system 301 that supports the implementations described herein. As shown in FIG. 3B, the system 301 includes many of the features of the system 300 of FIG. 3A (e.g., the smartphone 102, the tablet computing device 302, the network 308, and the server environment 310). The system 301 further includes an edge server 314. In the system 301, the smartphone 102 hosts an EMS digital assistant 104A, and the edge server 314 hosts an EMS digital assistant 104B, a patient charting application 306C and an ePCR data store 312C.

In some examples, the edge server 314 is a computing device configured to execute processor intensive operations that are sometimes involved when executing machine learning processes, such as NLP operations. Some implementations of the edge server 314 include, for example, one or more GPUs that are capable of efficiently executing matrix operations and substantial cache or other high-speed memory to service the GPUs. In some examples, the edge server 314 is a separate, ruggedized physical device that travels with EMS personnel in the field. In some examples, the edge server 314 is incorporated into other EMS field equipment such as a medical device and/or may be located in the EMS vehicle. Alternatively or additionally, the edge server 314 may be located within a carrying case for a medical device. In an implementation, the smartphone 102 and/or the tablet 302 may operate as the edge server 314 if the processing capability of these devices is sufficient to provide computing services associated with the edge server 314. The smartphone 102, the tablet 302, and the edge server 314 may all be local devices because the devices 102, 302, 314 are located in proximity to one another and to the EMS personnel and/or the emergency victim. The server environment 310 may be or include a remote device because the server environment 310 may be hosted in a cloud service comprising one or more cloud servers located remotely from all of the devices 102, 302, 314. The edge server 314 moves more computing capability into the local environment so that the computation intensive NLP models can run accurately and efficiently to support the digital assistant 104A even in the absence of a connection with the remote cloud server 310. In some instances, the smartphone 102 and/or the tablet 302 may lack the processing capability necessary to support these models.

Regardless of its physical form, the edge server 314 can be configured to interoperate with other devices of the system 301 directly or via the network 308. For instance, the edge server 314 can include a wireless network interface (e.g., a PAN interface, LAN interface, WAN interface, or the like) through which the edge server 314 can communicate with the smartphone 102, the tablet 302, and/or the server environment 310. In a reciprocal manner, the smartphone 102 and/or the tablet 302 may be configured to connect directly or indirectly to, and interoperate with the edge server 314, via a short-range wireless connection, such as a PAN connection or a LAN connection. In an implementation, the smartphone 102 and/or the tablet 302 may communicate via a short range wireless connection (e.g., network 308a) to the edge server 314 and, in turn, the edge serve 314 may communicate via a long range wireless connection (e.g., network 308b) to the server environment 310. The computer-implemented processes illustrated in FIG. 3B (e.g. the EMS digital assistant 104A, the EMS digital assistant 104B, the EMS digital assistant 304, and the patient charting applications 306A, 306B, and 306C) interoperate with one another over the connections described above via one or more APIs implemented by the processes.

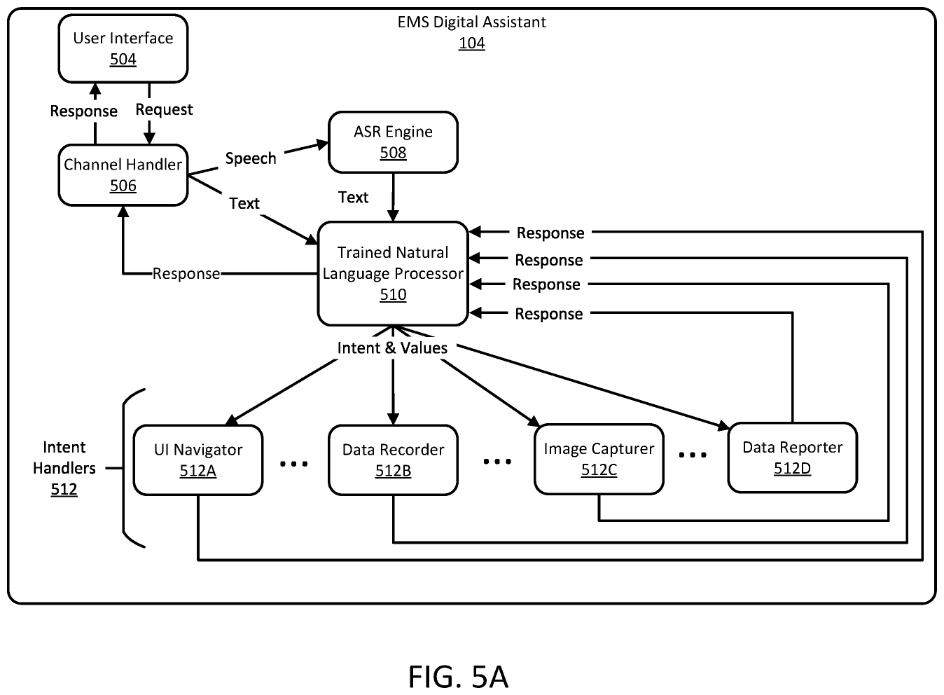

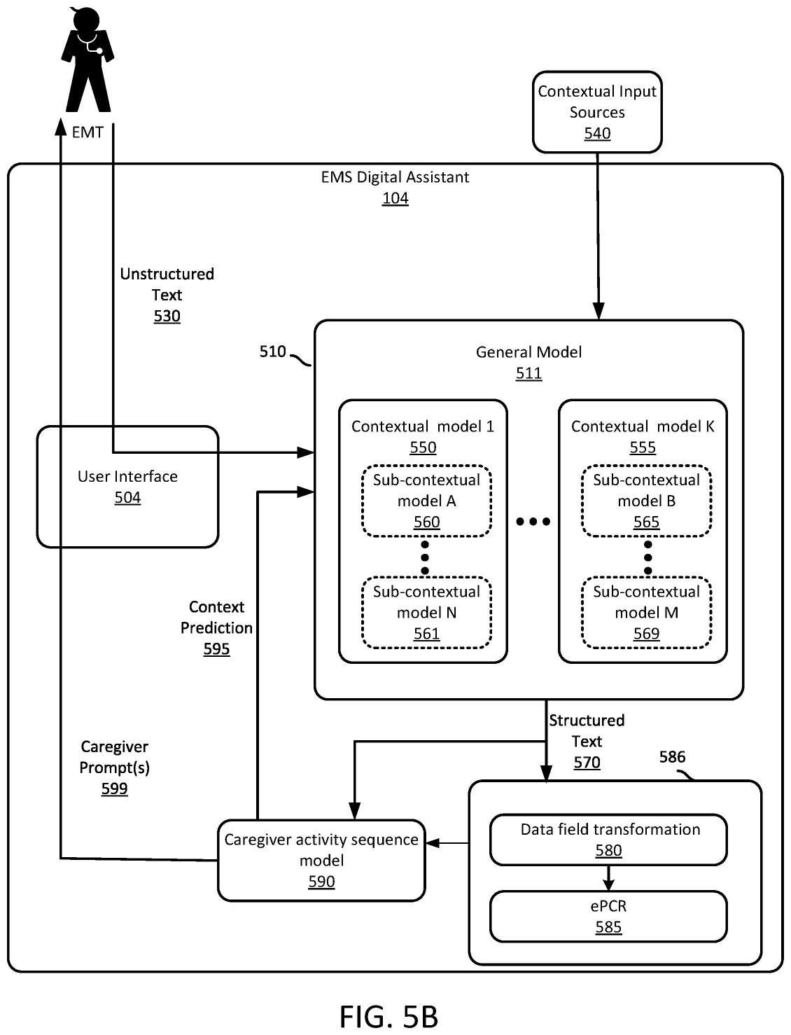

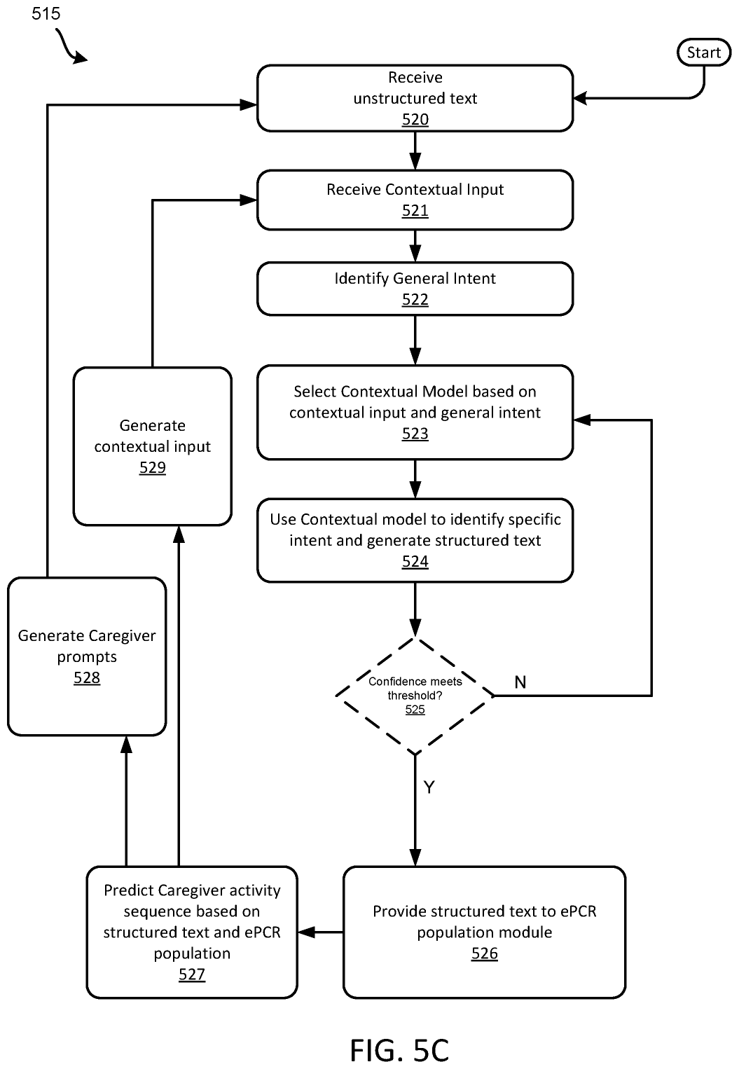

As shown in FIG. 3B, the EMS digital assistant 104A and the EMS digital assistant 104B are collectively configured to implement the EMS digital assistant 104 of FIG. 3A. In some examples, the EMS digital assistant 104B serves the EMS digital assistant 104A as a browser-based user interface to the smartphone 102. In these examples, the EMS digital assistant 104A is a thin client and relies on periodic communications with the EMS digital assistant 104B to operate properly. For instance, the EMS digital assistant 104A may rely on the EMS digital assistant 104B to execute some or all NLP processing, as is described further below with reference to FIGS. 5A-9. Moreover, in these examples, the data store of the EMS digital assistant 104A may be maintained in browser session storage and, thus, may contain a limited amount of data that is updated periodically with data from a data store of the EMS digital assistant 104B. In some examples, the EMS digital assistant 104A and/or 104B includes a service worker that caches data for subsequent transmission to the patient charting application 306B (e.g., periodically or in real-time if an operable network connection exists between the smartphone 102 and/or the edge server 314 and the remote environment 310). The data stores of the EMS digital assistant 104A and the EMS digital assistant 104B may exchange information periodically or in real time to maintain data currency.

In some examples, the EMS digital assistant 104A and/or the EMS digital assistant 104B is configured to transfer recorded ePCR data to remote data stores (e.g., the data store 312A, the data store 312B, and/or the data store 312C). The EMS digital assistant 104A and/or the EMS digital assistant 104A may be configured to execute this transfer in real time or in batches based on occurrence of one or more events (e.g., according to a time-based schedule, based on availability of sufficient network bandwidth, in response to caregiver input, etc.). This transfer may be effected by, for example, one or more API calls from the EMS digital assistant 104A and/or the EMS digital assistant 104B to the EMS digital assistant 304, the patient charting application 306A, the patient charting application 306B, the patient charting application 306C, the data store 312A, the data store 312B, and/or the data store 312C. For instance, in some examples, the EMS digital assistant 104A and/or the EMS digital assistant 104B transfers populated ePCR data fields to the data store 312A hosted by the tablet 302. Alternatively or additionally, in some examples, the EMS digital assistant 104A and/or the EMS digital assistant 104B transfers recorded ePCR data to the data store 312B hosted by the server environment 310. Alternatively or additionally, in some examples, the EMS digital assistant 104A and/or the EMS digital assistant 104B transfers recorded ePCR data to the data store 312C hosted by the edge server 314. In any case, transferred ePCR data can be accessed by the EMS digital assistant 104A and/or the EMS digital assistant 104B, the EMS digital assistant 304, the patient charting application 306A, the patient charting application 306B, and/or the patient charting application 306C. In this way, the system 301 enables the charting application 306A, the charting application 306B, and/or the charting application 306C to access ePCR data fields populated by the EMS digital assistant 104A and/or the EMS digital assistant 104B. This access, in turn, enables the charting application 306A, the charting application 306B, and/or the charting application 306C to interact with a caregiver (e.g., the caregiver 106 of FIG. 1) to complete or review administrative portions of an ePCR.

It should be noted that the charting applications 306A, 306B, and/or 306C and the data stores 312A, 312B, and 312C can be configured to operate collaboratively or independently, depending on the design goals of a particular installation and the current operating environment. For instance, in some examples, the charting application 306B serves the charting application 306A as a browser-based user interface to the tablet 302. Alternatively or additionally, in some examples, the charting application 306C serves the charting application 306A as a browser-based user interface to the tablet 302. In these examples, the charting application 306A is a thin client and relies on periodic communications with the charting applications 306B and/or the charting application 306C to operate properly. Moreover, in these examples, the data store 312A may be maintained in browser session storage and, thus, may contain a limited amount of data that is updated periodically with data from the data stores 312B and/or 312C. Alternatively or additionally, in some examples, the charting application 306A is an independent application configured to execute natively under an operating system of the tablet 302. In these examples, the data store 312A may contain all of the data needed for the charting application 306A to operate properly. In any case, it should be noted that the data stores 312A, 312B, and 312C may exchange information periodically or in real time to maintain data currency.

The additional computing resources provided by the edge server 314 can add several capabilities to the system 301. For instance, in some implementations, the edge server 314 enables the smartphone 102, the tablet 302, and the edge server 314 to tolerate faults and operate robustly in the face of an inoperable WAN connection to the server environment 310. For instance, in some examples, the patient charting application 306A is configured to interoperate with the patient charting application 306C by default and the patient charting application 306C or the data store 312C is configured to replicate data from the data store 312C to the data store 312B when an operable WAN connection to the server environment 310 is available. This implementation tolerates WAN connection faults well because the patient charting application 306C can store ePCR data in the ePCR data store 312 for extended WAN connection outage periods and relay the stored ePCR data to the ePCR data store 312B when the WAN connection becomes available. Other approaches to establishing a high-available and fault tolerant system that the edge server 314 enables will be apparent in view of this disclosure. For instance, in some examples, the edge server 314 operates as a proxy server and can failover from the patient charting application 306B to the patient charting application 306C upon detection of a WAN connection fault.

Other advantages realized via the edge server 314 include faster and more accurate execution of NLP processes and less latency in data availability between instances of the EMS digital assistant 104A, the EMS digital assistant 304, and the patient charting application 306A. These benefits are realized by virtue of the edge server's powerful hardware and central storage and synchronization of EMS digital assistant and ePCR data. Some implementations that leverage these distributed processing advantages are described further below with reference to FIGS. 5A-9.

It should be noted that some implementations of the system 300 can be configured to convert to the system 301 upon introduction and detection of the edge server 314 by any of the processes of the system 300.

Alternatively or additionally, in some examples, the EMS digital assistant 104A is an independent application configured to execute natively under an operating system of the smartphone 102. In these examples, processing capability of the smartphone 102 and/or the tablet 302 may be sufficient to provide the computing power necessary for NLP models and/or the NLP models may execute in a streamlined manner so as to reduce the computational complexity but retain accuracy.

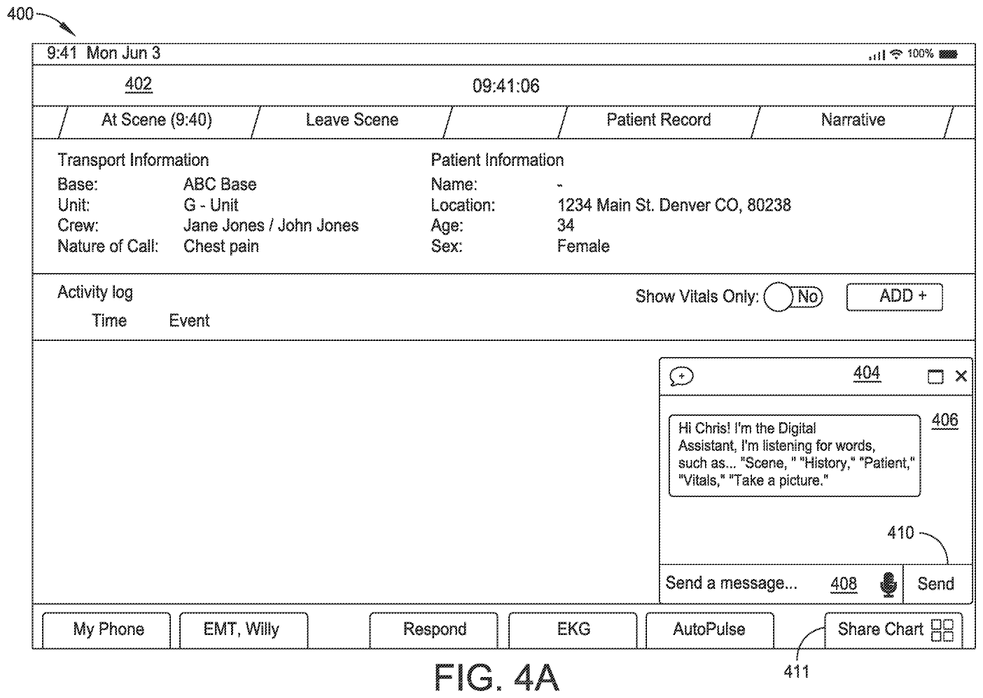

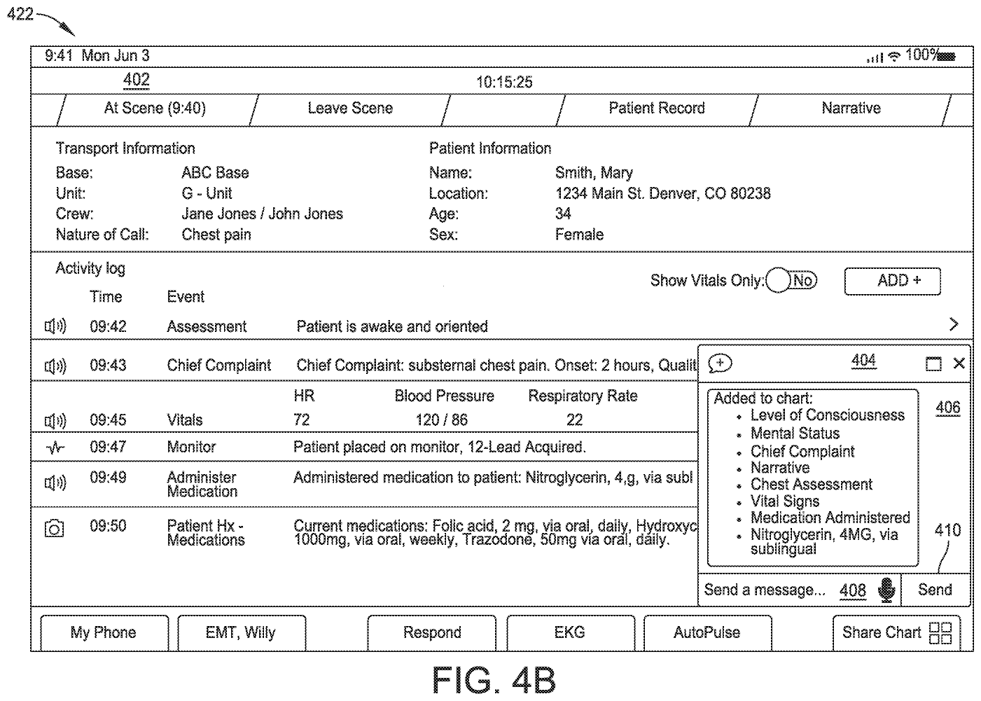

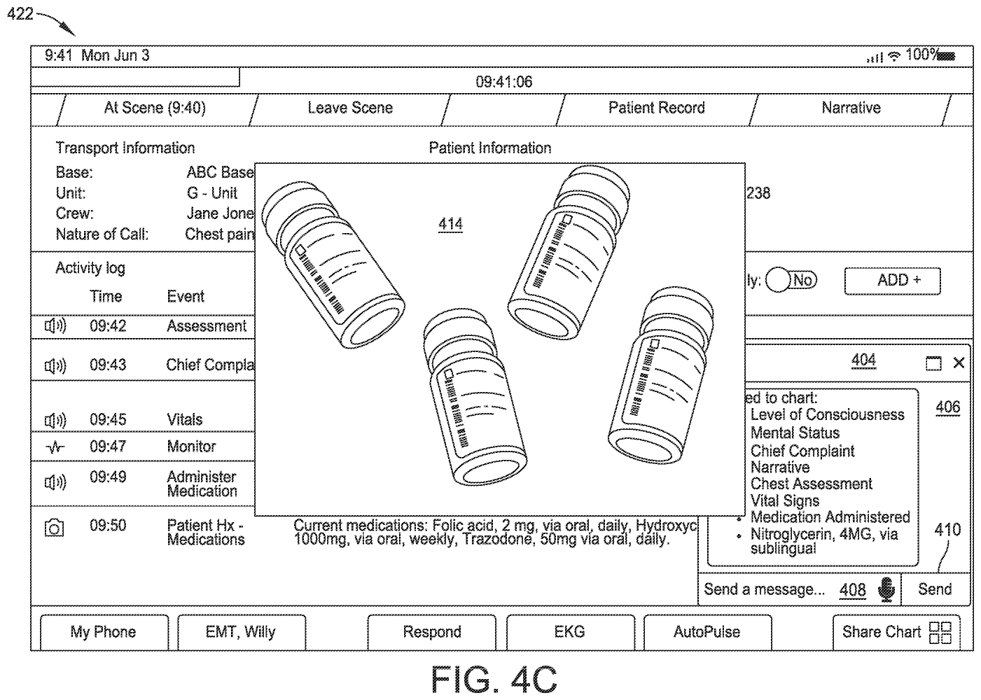

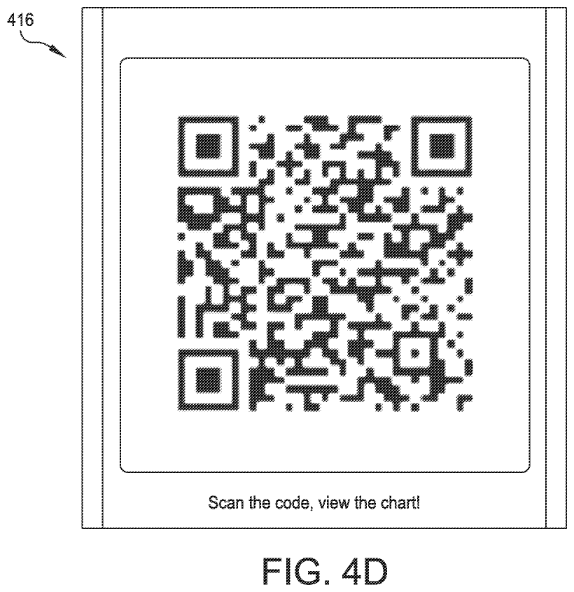

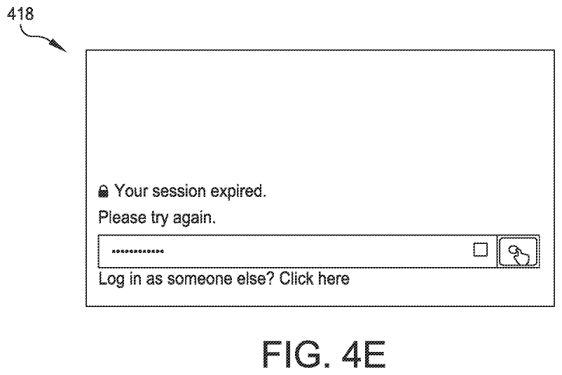

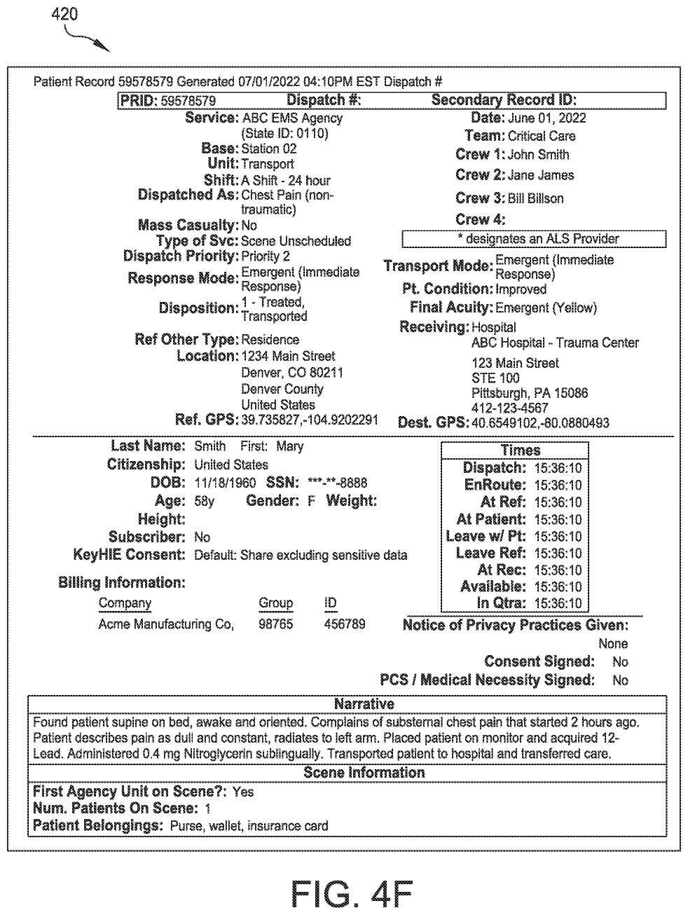

FIGS. 4A through 4F illustrate operations executed by a charting application (e.g., the patient charting application 306A and/or the patient charting application 306B of FIG. 3A or 3B) and a digital assistant (e.g., the EMS digital assistant 304 of FIG. 3A or 3B) relative to transferred ePCR data fields. For instance, FIG. 4A illustrates a user interface screen 400 displayed by the charting application and the digital assistant subsequent to initialization. The screen 400 includes a chart window 402 that is displayed by the charting application and a chat window 404 displayed by the digital assistant. The chat window 404 includes a conversation control 406, a message input control 408, a send control 410, and a share chart control 411. The conversation control 406 is configured to display communications between the digital assistant and a caregiver (e.g., the caregiver 106 of FIG. 1). The message control 408 is configured to receive voice and/or text input from the caregiver. The send control 410 and the chart control 411 are each configured to receive tactile input. The digital assistant is also configured to post, in response to reception of tactile input via the send control 410, input received by the message control 408 to the conversation control 406 for processing by the digital assistant. As shown in screen 400, the digital assistant has initiated a conversation with a caregiver by posting a message including recognizable words to the conversation control 406.

FIG. 4B illustrates a user interface screen 422 displayed by the charting application and the digital assistant subsequent to both applications gaining access to transferred ePCR data, such as that recorded by the EMS digital assistant 104 of FIG. 1. In some examples, the digital assistant gains access to the transferred ePCR data fields by scanning an image of an identifier of a patient encounter, such as the QR code displayed in the QR control 248 of FIG. 2I, decoding the identifier from the image, and requesting the transferred ePCR data fields associated with the identifier from a data store (e.g., the data store 312A, 312B, and/or 312C).

As shown in FIG. 4B, the screen 422 includes the windows 402 and 404 of FIG. 4A, however, in the screen 422 the conversation control 406 includes a message listing an audit trail of ePCR data fields transferred to the charting application. Moreover, in the screen 422 the window 402 displays populated ePCR data fields that are accessible and editable by the charting application. As shown, these ePCR data fields contain data gathered by the EMS digital assistant 104 during the encounter 100 described above with reference to FIG. 1. As illustrated in FIG. 4C, these ePCR data fields can include an image 414 of the medication 120 of FIG. 1.

Returning to FIG. 4A, the digital assistant is configured to receive tactile input via the chart control 411 and, in response thereto, to generate a unique identifier of the ePCR, encode the identifier into a QR code, and display the QR code within a QR code control, as illustrated in screen 416 of FIG. 4D. In some examples, the identifier of the ePCR can include an API endpoint, such as a uniform resource identifier, implemented by the charting application that provides secure access to a copy of the ePCR. In these examples, to access the copy of the ePCR, a caregiver must provide security credentials to the charting application via a screen such as screen 418 of FIG. 4E. Where the security credentials are authentic and authorized, the charting application provides the copy of the ePCR, as shown in screen 420 of FIG. 4F.

As can be seen from the examples described above, an EMS digital assistant 104 can provide a caregiver with a variety of useful functionality. However, in some situations, a caregiver may wish to configure an EMS digital assistant to interact with the caregiver in a particular manner. For instance, when in a chaotic environment, the caregiver may wish to minimize the number of direct interactions requested by the EMS digital assistant and/or may wish to that the EMS digital assistant only confirm observations, rather than request new additional information. As another example, where the caregiver is addressing an unfamiliar or critical medical condition, the caregiver may wish for the EMS digital assistant to prompt the caregiver for actions in accord with an established treatment protocol.

Given the variety of types of interaction that may be preferable to a caregiver, some examples of a digital assistant (e.g., the EMS digital assistants 104 and/or 304 of FIG. 3A or 3B) are configured to implement any of a variety of operational interactivity modes. For instance, in some examples, the EMS digital assistant is configured to operate in a user-driven mode, a predictive mode, an observation mode, a confirmation mode, and/or a conversation mode.

When configured to operate in user-driven mode, the digital assistant follows express commands from a caregiver. In some implementations, these express commands may enable the caregiver to navigate the user interface for data entry to the ePCR and/or to recall information from previously entered data. Examples of commands that the digital assistant is configured to execute while in user-driven mode include commands to record ePCR data, commands to navigate to particular fields within ePCR data, commands to control the computing device hosting the EMS digital assistant, commands to provide notifications regarding ePCR data to the caregiver or others now or in the future, and the like. Table 3 lists some examples of commands recognizable by the digital assistant and responses thereto.

TABLE 3

| Command | Digital Assistant Response |
|---|---|
| “Take me to the | Displays one or more UI controls |
| vitals section” | configured to accept vital signs |
| “What were vitals | Displays one or more UI controls |
| 5 minutes ago” | configured to display vital signs as of 5 |
| | minutes in the past and/or provides audible |
| | vital sign data |
| “Tell me the pulse of | Sounds a notification of the |
| the patient every minute | patient's pulse as |
| for the next ten” | recorded in local ePCR data |
| “Show me current | Displays and/or provides audible |
| medications” | medication information |

When configured to operate in predictive mode, the digital assistant observes the environment, forecasts parts of the ePCR that are likely to help the caregiver and navigates to those portions. Table 4 lists some examples of observations recognizable by the digital assistant and responses thereto.

TABLE 4

| Observation | Digital Assistant Response |
|---|---|
| Speech stating “patient | Navigate to monitor setup |
| presents with chest pain” | and ECG strip entry |
| Speech stating “patient | Navigate to respiratory |
| presents hypoxia” | system parameter entry |

When configured to operate in observational mode, the digital assistant observes the environment and records ePCR data but does not interact with the caregiver. Table 5 lists some examples of observations recognizable by the digital assistant and responses thereto.

TABLE 5

| Observation | Digital Assistant Response |
|---|---|
| Speech including wake-up word | Enter observational mode |
| Speech stating “BP | Record ePCR data indicating that |
| is 120/80” | the patient's blood pressure |
| | is 120/80 at the current time |
| Speech stating “get | Record ePCR data with a code that |
| him on a stretcher” | indicates patient was moved to the |
| | ambulance via a stretcher |

When configured to operate in confirmation mode, the digital assistant observes the environment and records ePCR data but does not interact with the caregiver other than to confirm observations. These confirmations can be auditory, visual, tactile, etc.

When configured to operate in conversational mode, the digital assistant observes the environment, records ePCR data, and interacts with the caregiver to resolve any ambiguities in the observations, and/or to provide information. Table 6 lists some examples of observations recognizable by the digital assistant and responses thereto.

TABLE 6

| Observation | Digital Assistant Response |
|---|---|
| Speech stating | Prompt with “What gauge of needle was |
| “IV established” | used?” |
| Speech stating | Record ePCR data indicating that the IV |
| “18” | was established with an 18-gauge needle at |
| | the present time |
| Speech stating | Prompt with “What was the tube size?” |
| “patent intubated” | “What was the intubation depth?” “Who |
| | confirmed success?” “How was success |
| | confirmed?” |
| Speech stating | Enters observational mode |
| “later” | |

It should be noted that the EMS digital assistant 104 can switch between the interactivity modes introduced above autonomously, depending on the intents expressed by the caregiver and/or based on environmental observations. For instance, in at least one example, the EMS digital assistant is configured to monitor the ambient noise level and, where the noise level exceeds a threshold value, automatically switch to a mode preferred by the caregiver for chaotic environments (e.g., observational mode). It should also be noted that the EMS digital assistant may assume several of the interactivity modes during a single patient encounter. Alternatively, in some examples, the caregiver can configure the EMS digital assistant to operate solely within one or more default modes, based on the preferences of the caregiver.

In an implementation, the EMS digital assistant 104 may calculate a chaos score based on the ambient background noise level as indicated by the audio input. The EMS digital assistant 104 may operate in a default or predetermined fallback interactivity mode when the chaos score exceeds the threshold value. For example, the EMS digital assistant 104 may identify the observational mode as the predetermined fallback interactivity mode and automatically switch from a conversational mode, for example, to the observational mode when the chaos score exceeds the threshold. Similarly, the EMS digital assistant 104 may resume the conversational mode when the chaos score drops below the threshold value. In the observational mode with a high chaos score, the EMS digital assistant 104 may record all of the data from the encounter and operate the trained NLP processor on this recorded data. Further, the EMS digital assistant 104 may automatically switch from a verbal and/or visual feedback mode for caregiver prompts to a haptic mode. In an implementation, the EMS digital assistant 104 may evaluate the duration of a chaos score. For example, the audible noise may increase temporarily due to a siren, a stretcher rumble, a scream, etc. to name a few examples of shorter duration noises. The EMS digital assistant 104 may, in some cases, remain in a particular mode and just postpone audible interactions until a high chaos score of a shorter duration subsides. Further, the EMS digital assistant 104 may record and identify sounds during a high chaos score and use this information as contextual input for the trained NLP model 104 (e.g., as described in regard to FIG. 5B) and as contextual input for generating caregiver prompts. In an implementation, the EMS digital assistant 104 may analyze the audio recording to discriminate between unstructured text relevant to patient care and the ambient noise.

FIG. 5A is a block diagram of one implementation of the EMS digital assistant 104 of FIG. 1. As shown, the EMS digital assistant 104 includes a user interface 504, a channel handler 506, an ASR engine 508, a trained NLP 510, and intent handlers 512.

In some examples, the user interface 504 is configured to interoperate with devices that make up the physical user interface of the computing device that hosts the EMS digital assistant 104. For instance, in one example, these physical user interface devices include the touchscreen, the microphone, and the speaker of the smartphone 102 of FIG. 1. Moreover, in some examples, the user interface 504 is configured to receive input from the physical user interface devices and to render output via the physical user interface devices. Each physical user interface device used for communication with a caregiver may be associated with a channel. Input data received via a channel can specify inbound communications from a caregiver. In some examples, the user interface 504 is configured to transmit requests that include input data received via a channel and an identifier of the channel to the channel hander 506 for processing. Output rendered via a channel can articulate outbound responses for the caregiver. In some examples, the user interface 504 is configured to receive responses from the channel handler 506 and to render output data included therein via a channel identified in the response.

In certain examples, the channel handler 506 is configured to process requests received from the user interface 504 and responses received from the NLP 510. In these examples, to process a request the handler 506 is configured to generate a communication identifier, store an association between the communication identifier and the channel identifier received in the request, and identify a type of the input data (text, audio, etc.) stored in the request. The handler 506 is further configured to transmit the communication identifier and the input data specified in the request to either the ASR engine 508 (i.e., where the input data is audio data) or the NLP 510 (i.e., where the input data is text data).

In some examples, to process a response received from the NLP 510, the handler 506 is configured to identify a channel identifier associated with the communication identifier specified in the response, generate output data based on a type of channel (audio, visual, etc.) identified by the channel identifier and the text specified in the response, and transmit a response to the user interface 504 that includes the channel identifier and the output data. It should be noted that, in some examples, the channel handler 506 is configured to render audio that articulates human speech when generating output data for a channel associated with an audio device, such as a speaker. It should also be noted that, in some examples, the channel hander 506 is configured to generate output data and transmit responses on multiple channels (e.g., both audio and visual) either generally or in response to certain requests.

Continuing with FIG. 5A, the ASR engine 508 is configured to receive the communication identifier and the audio data from the handler 506 and to process the same. In some examples, this processing includes rendering text data from speech recognizable within the audio data. In some examples, the ASR engine 508 renders the text data from the audio data by executing an ASR process (for example, but not limited to, Apple Dictation, Google Gboard, Nuance Dragon Anywhere, Amazon Transcribe, Microsoft Azure Speech to Text, IBM Watson Speech to Text, Windows 10 Speech Recognition, etc.). The processing that the ASR engine 508 is configured to execute can further include transmitting the text data and the communication identifier to the NLP 510.

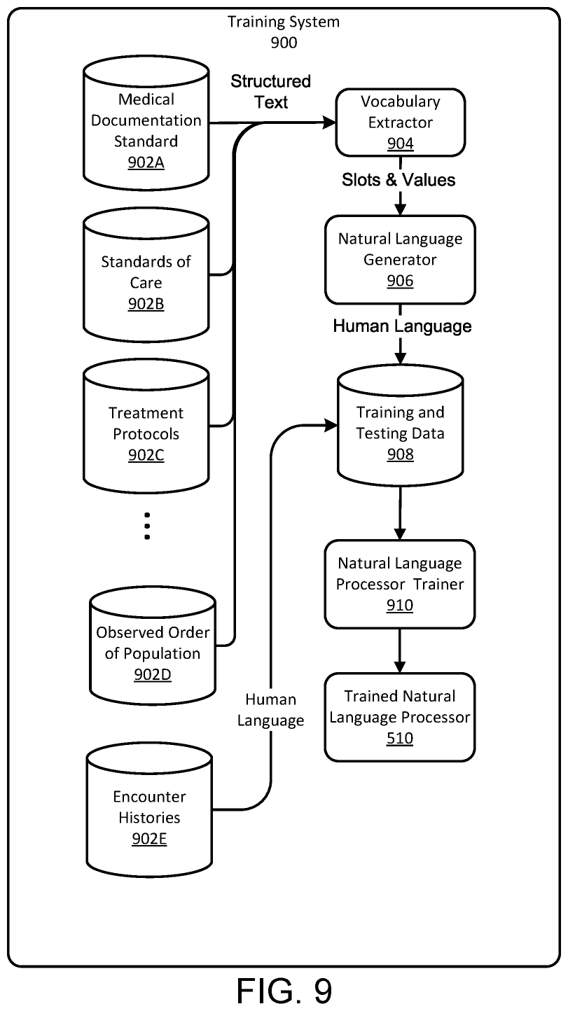

Continuing with FIG. 5A, the NLP 510 is configured to process a communication identifier and text data received from either the channel handler 506 or the ASR engine 508 and to respond to the handler 506 based on responses received from the intent handlers 512. In some examples, this processing includes receiving the communication identifier and the text data and extracting one or more intents and one or more associated values articulated within the text data. In some examples, the NLP 510 extracts intents and values specified within the text data by applying one or more specialized natural language processing models trained to understand medical terminology, syntax, and grammar utilized by caregivers. In certain examples, these natural language processing models are trained machine learning models based on a data science and machine learning platform as described further below with reference to FIG. 9. In some examples, the NLP 510 awaits a wakeup word to begin applying the natural language processing models to inbound text data. Further, in some examples, the natural language processing models produce a metric that indicates a confidence that the extracted intents and values have been correctly identified. In certain examples, the NLP 510 is configured to abort processing where the confidence metric is below a threshold value. Further, in these examples, the NLP 510 may generate a response without output text indicating that the EMS digital assistant 104 was unable to understand the last input.

In some examples, the processing that the NLP 510 is configured to execute can further include passing the values associated with the extracted intents in calls to one or more of the intent handlers 512 associated with the intents. These calls can be associated with the communication identifier received with the text data from which the intents are extracted. To process a message text generated by a call to an intent handler 512, the NLP 510 is configured to receive the message text, generate a response that includes the message text and the communication identifier associated with the call, and transmit the response to the channel handler 506.

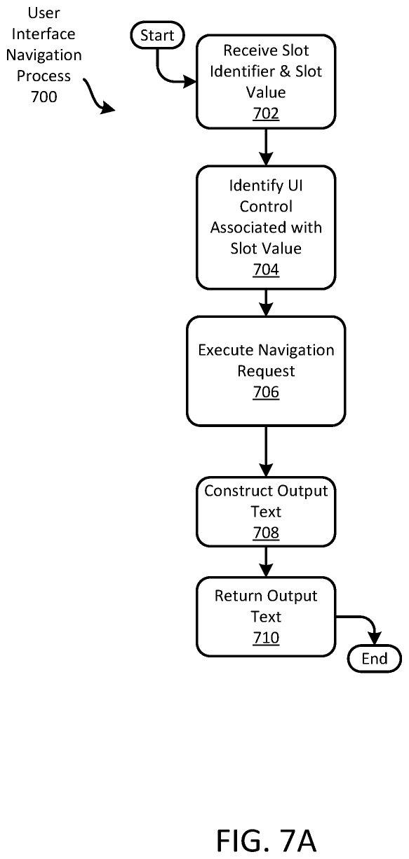

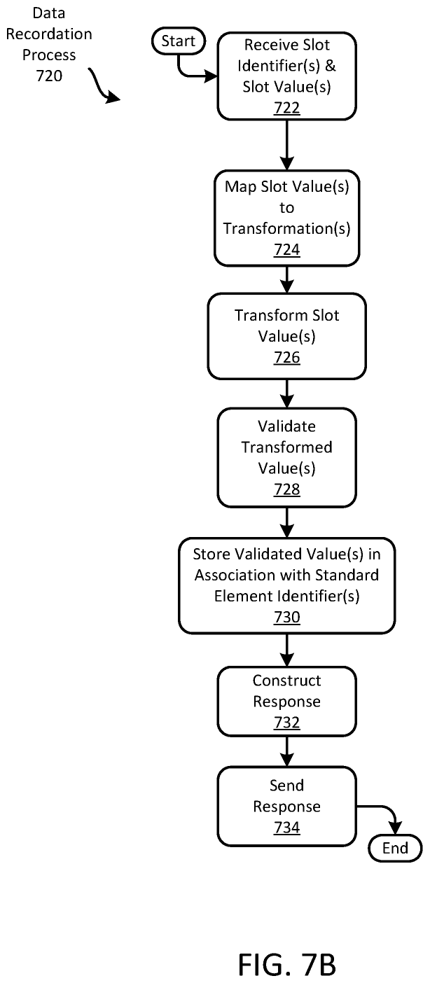

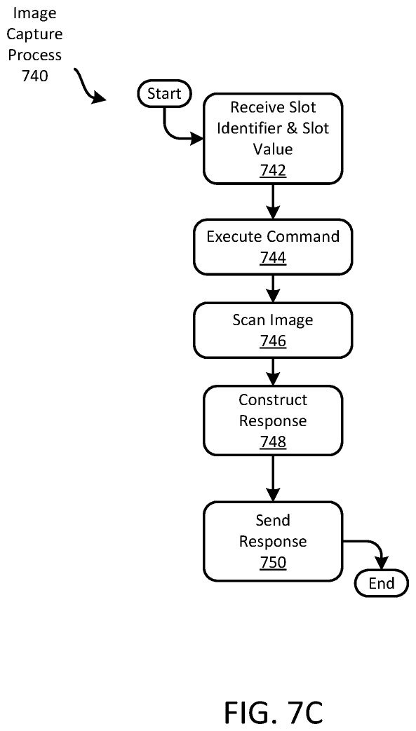

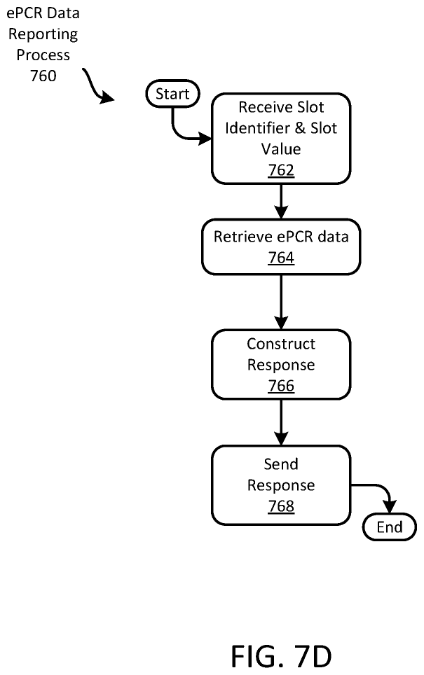

Continuing with FIG. 5A, a wide variety of intent handlers 512 can be included in various examples. In general, the intent handlers 512 are configured to receive values from the NLP 510, execute some useful automation that is responsive to the intent based on the values, and transmit message text relevant to the executed automation to the NLP 510 for further processing. The message text can articulate a message to be rendered to a caregiver. The example illustrated in FIG. 5A includes four intent handlers—a user interface navigator 512A, a data recorder 512B, an image capturer 512C, and a data reporter 512D. The navigator 512A is configured to interoperate with a charting application (e.g., the patient charting application 306A and/or the patient charting application 306B) to cause the charting application to display a user interface control specified by a value passed to the navigator 512A. One example of a process that the navigator 512A is configured to execute is described below with reference to FIG. 7A. The recorder 512B is configured to record ePCR data specified by one or more values passed to the recorder 512B. One example of a process that the recorder 512B is configured to execute is described below with reference to FIG. 7B. The capturer 512C is configured to interoperate with a computing device hosting the digital assistant 104 to cause the computing device to capture an image. One example of a process that the capturer 512C is configured to execute is described below with reference to FIG. 7C. The reporter 512D is configured to report previously recorded ePCR data to the caregiver. One example of a process that the reporter 512D is configured to execute is described below with reference to FIG. 7D. Many other intent handlers are possible, and the scope of this disclosure is not limited to the specific intent handlers 512 described herein.

Within implementations that include an edge server (e.g., the edge server 314 of FIG. 3B), the processes executed by the EMS digital assistant 104 illustrated in FIG. 5A can be distributed across a first EMS digital assistant (e.g., the EMS digital assistant 104A of FIG. 3B) hosted by a first device and a second EMS digital assistant (e.g., the EMS digital assistant 104B of FIG. 3B) hosted by the edge server. For instance, in some examples, the first EMS digital assistant executes the user interface 504, and the second EMS digital assistant executes the remaining processes. In another example, the first EMS digital assistant executes the user interface 504 and the channel handler 506, and the second EMS digital assistant executes the remaining processes. Both of these examples advantageously leverage the edge server's ability to efficiently execute compute-intensive machine learning operations.

In an implementation with an edge server, various tasks may be relegated to one or the other of the smartphone and the edge server. For example, navigation to particular ePCR fields and/or recognition of keywords may be relegated to the smartphone or tablet processor. On the other hand, predictions, either with regard to the ePCR field population or clinical guidance, image recognition, object detection, and interpretation of streams of conversation may be relegated to the edge server processor. The streams of conversation require more complex models to recognize sentence structure and/or grammar and may be better served by the processing capability of the edge server than the smartphone or tablet.

Referring to FIGS. 5B and 5C, schematic illustrations of examples of reciprocal modifications in the functioning of the EMS digital assistant and the caregiver workflow are shown. The EMS digital assistant 104 provides the user interface 504 and the trained natural language processor (NLP) 510. As discussed herein, the trained NLP 510 receives unstructured text 530 (e.g., verbal input converted to text and/or textual input) from the caregiver 106. The trained NLP 510 converts the unstructured text 530 to structured text 570 via application of one or more models and provides the structured text 570 to an ePCR population module 586. It should be noted that, in some examples, the ePCR population module 586 is implemented as one or more intent handlers 512. The ePCR population module 586 transforms 580 and maps the structured text to data fields of the ePCR 585 according to the particular schema of the ePCR 585. Additionally, the ePCR population module 586 provides the structured text 570 and/or information about the correlation of this text to the data fields (e.g., from the data field transformation 580) to a caregiver activity sequence model 590. The model 590 enables the EMS digital assistant 104 to generate caregiver prompts 599 and generate context predictions 595 based on the recognized values in the structured text 570. The model 590 identifies procedurally related caregiver activities along with procedurally related ePCR data fields to predict future caregiver activities and generate appropriate prompts for these activities. The prompts 599 may include instructions to perform procedurally related tasks and/or may provide procedurally related patient or encounter information. These prompts can be verbal and/or visual, depending on the modality of the user interface 504 provided by the EMS digital assistant 104. Further, the model 590 provides the context prediction 595 back to the trained NLP 510.

The trained NLP 510 may include, for example, a general model (e.g., the general model 511), one or more contextual models (e.g., the contextual models 550-555), and/or one or more sub-contextual models (e.g., the sub-contextual models 560-569). Further, the trained NLP 510 may receive contextual input from external contextual input sources 540 and from the context prediction 595 from the model 590. For example, the external contextual input sources 540 may include a GPS and/or cellular location device (e.g., the positioning system 1040 of FIG. 10A or 10B) and/or other devices and/or applications associated with the mobile device 102 (e.g., a clock, a camera, a microphone, a navigation application, a calendar application, a dispatch application, a billing application, etc.). The model 590 may generate the context prediction 595 based on procedural relationships between caregiver activities and/or between data fields in the ePCR 585. As one example, the structured text 570 from the trained NLP 510 may be “blood pressure” with “160 systolic” and “90 diastolic.” The model 590 may combine this with other data indicating a location at a senior center or a nightclub along with other patient demographic data and/or medical data in the ePCR 585 to predict next steps of a chronic heart condition or a drug overdose. As a further example, the EMS digital assistant 104 may monitor the ePCR 585 and use the model 590 to identify missing data and generate prompts to solicit or query for the missing data from caregivers and/or or other devices. As another example, the model 590 may include rules based on physiological facts (e.g., pregnant=female and/or pregnant male), medical treatment protocols, and/or machine learning that may identify missing or inferable data fields. The prompts 599 may include requests for confirmation of inferred data. As a further example, the medical treatment protocols may specify specific transport conditions for trauma or specific examination procedures for a bleeding head wound. The model 590 may predict procedures and context and generate prompts based on these conditions. As yet another example, in an implementation, the model 590 may identify procedural relationships from the structured text 570 based on one or more medical protocols. For example, the caregiver 106 may record the observations “mobile,” “no pain,” and “walking” in the ePCR 585 for a trauma victim. The model 590 may predict a context 595 including “no spinal immobilization,” “no backboard,” and “seated” based on the recorded observations. These data field values correspond to medical protocols which indicate that a mobile walking patient that is not reporting pain can be transported without spinal immobilization, without a backboard, and in a seated position. The model 590 may also link data fields according to ICD codes associated with the data fields. The model 590 may then generate appropriate prompts 599 based on the predicted sequence for the trauma patient. As another example, the model 590 may correlate medications and conditions and determine a probability that a medication indicates a particular condition. As an example, if this probability is 99% or higher, the model 590 may generate a prompt indicating a likelihood of a particular condition and/or interventions for the indicated conditions based on structured text indicating the medication. If the probability is between 80-99%, the model 590 may prompt the caregiver to ask the patient or a bystander or consult a medical record to confirm the condition. In an implementation, the EMS digital assistant 104 may access and search a victim's medical record as received from a medical record database (e.g., the database 1005 in FIG. 10A or 10B) for the condition and/or the medication.

In an implementation, the general model 511 may function as a state machine that follows a pre-determined path to convert from unstructured text 530 to structured text 570. The pre-determined path may depend on the contextual input.

Alternatively, the general model 511 may orchestrate, direct, and/or coordinate a selection of one or more model(s) applied to the unstructured text 530 based on the contextual input. This contextual input may progressively change over the course of operations of the EMS digital assistant 104 as the caregiver activities proceed, as the ePCR becomes populated, and/or as external context changes. Due to these progressive changes, the interaction between the EMS digital assistant 104 and the caregiver 106 that both guides and assists the caregiver and improves the function of the EMS digital assistant 104 is an ongoing interaction. The general model 511 may identify an intent based on the unstructured text 530 and may then select a contextual model (e.g., contextual model 550, . . . , contextual model 555) and, optionally, a sub-contextual model (sub-contextual model 560, . . . , sub-contextual model 560 and sub-contextual model 565, . . . , sub-contextual model 569), to more efficiently and accurately interpret and understand the unstructured text 530. The general model 511 may evaluate the confidence of intentions and structured text identification to evaluate the model selection. If the confidence is below a certain threshold, the general model may re-select or re-combine various sub-models to re-generate the output and improve the confidence associated with the structured text. Vocabulary, syntax, and/or text structure may vary between contexts and the more refined and tailored to the specific context the model is, the more efficiently and accurately the model can generate the structured text 570. For example, one or more of the sentence subject, verb, numerical variables and constants, etc. may vary in meaning and structure from context to context. Thus the general model 511 may determine a general intent and text values and, based on this, hand off to one or more specific models (e.g., based on the specificity of the intent) to determine the structured text. In turn, the general model 511 can hand off the structured text to the model 590 for predictions of next steps for the caregiver and of the current or upcoming context. The next iteration of the general model 511 may apply the predicted context to anticipate and/or prioritize the next contextual, and optionally, sub-contextual, models for the next set of unstructured text input.

As one more specific example, with an identified intent of a cardiac assessment, the general model 511 may hand off to a contextual model for cardiac assessment. The contextual model for cardiac assessment may, upon receipt of unstructured data indicating an intent of a defibrillation, hand off to a sub-contextual model for an arrhythmogenic cardiac arrest as opposed to a sub-contextual model for a non-arrhythmogenic cardiac arrest. Additionally, with an identified intent of cardiac assessment along with an intent for trauma intervention, the general model 511 may invoke specific combinations of models to handle the specific mix of unstructured text. In some implementations, the model selection may occur on demand at the point of care and/or may be previously provisioned. For example, the general model 511 may be provisioned to utilize particular sub-models for patient conditions and/or EMS operations typically seen by a particular agency or transport crew. Additionally, the general model 511 may be configured to recognize an unexpected patient condition and/or EMS operations and identify and utilize a different sub-model.

The contextual and sub-contextual models reflect specific contexts in terms of at least geo-location, modality of care, protocols, historic patterns of care, type of EMS service, a type or nature of service, etc. For example, the NLP models for structured text related to drowning may vary from a northern climate where cold-water drownings are likely to a southern climate where cold-water drownings are unusual. As another example, the NLP models may be different for a small rural EMS agency that primarily deploys a few helicopters as opposed to a large urban EMS agency with a fleet of ambulances. As a further example, the context of a call to an emergency scene may be different and require a different NLP model than the context of a call to transfer a patient between facilities. The geo-locations of the transport vehicles in these two situations may enable a distinction between these two contexts.

Within implementations that include an edge server (e.g., the edge server 314 of FIG. 3B), the processes executed by the EMS digital assistant 104 illustrated in FIG. 5B can be distributed across a first EMS digital assistant (e.g., the EMS digital assistant 104A of FIG. 3B) hosted by a first device and a second EMS digital assistant (e.g., the EMS digital assistant 104B of FIG. 3B) hosted by the edge server. For instance, in some examples, the first EMS digital assistant executes the user interface 504 and collects input from the contextual sources 540, and the second EMS digital assistant executes the remaining processes. In another example, the first EMS digital assistant executes the user interface 504, collects input from the contextual sources 540 and executes the general model 511, and the second EMS digital assistant executes the remaining processes. Both of these examples advantageously leverage the edge server's ability to efficiently execute compute-intensive machine learning operations, however, the latter example enables the first EMS digital assistant to deal with easily recognized human language (e.g., intents that are directed to device operation rather than specialized, complex medical procedures and nomenclature).

Referring to FIG. 5C, an example of a method 515 of implementing the reciprocal modifications in the functioning of the EMS digital assistant and the caregiver workflow is shown. In this method, the trained NLP 510 receives unstructured text at the stage 520. At the stage 521, the trained NLP 510 receives both external contextual input (e.g., from the contextual input sources 540) and contextual input generated based on a predicted caregiver activity sequence (e.g., as generated at the stage 529 during iterations of the method 515 beyond an initial iteration). The general model 511 identifies a general intent 522 and selects or identifies a contextual model, and, optionally, a sub-contextual model, at the stage 523 based on the generalized intent identified at the stage 522 and based on the contextual input from the stage 521. In an implementation, the general model 511 may invoke multiple contextual models and/or combinations thereof at the stage 523. At the stage 524, the contextual or sub-contextual model(s) identify specific intents to generate the structured text 570. Optionally, at the stage 525, the general model 511 may evaluate a confidence of the structured text 570 as determined by the contextual or sub-contextual model(s). If this confidence fails to meet a pre-determined threshold, then the general model 511 may re-allocate the unstructured text to a different contextual model(s), sub-contextual model(s), or combination thereof. The general model 511 may repeat this procedure until the structured text confidence exceeds the threshold. Once the structured text confidence exceeds the threshold, the trained NLP 510 may provide the structured text to the ePCR population module 586 at the stage 526. In addition to populating the ePCR 585 (e.g., as shown in FIG. 5B), the ePCR population module 586 provides the ePCR population information to the caregiver activity sequence model 590. At the stage 527, the model 590 predicts a caregiver activity sequence based on the structured text and procedural relationships between caregiver activities and ePCR data fields. Further, at the stage 529, the model 590 predicts a context for current and/or subsequent activity and generated contextual input. The contextual input may be based on one or more of the predicted caregiver activity sequence, the populated and/or unpopulated (i.e., fields lacking data entry) ePCR data fields, and/or procedural relationships between populated and/or unpopulated ePCR data fields. At the stage 528, the model 590 generates caregiver prompts based on the structured text outputs from the trained NLP model 510. In this manner, the trained NLP model 510 provides ongoing guidance and, in some cases, modification of caregiver activities in providing care to the patient based on the structured text output.

Within implementations that include an edge server (e.g., the edge server 314 of FIG. 3B), the processes executed by the EMS digital assistant 104 illustrated in FIG. 5C can be distributed across a first EMS digital assistant (e.g., the EMS digital assistant 104A of FIG. 3B) hosted by a first device and a second EMS digital assistant (e.g., the EMS digital assistant 104B of FIG. 3B) hosted by the edge server. For instance, in some examples, the first EMS digital assistant executes the operations 520 and 521, and the second EMS digital assistant executes the remaining operations. In another example, the first EMS digital assistant executes the operations 520-522, and the second EMS digital assistant executes the remaining processes. Both of these examples advantageously leverage the edge server's ability to efficiently execute compute-intensive machine learning operations, however, the latter example enables the first EMS digital assistant to deal with easily recognized human language (e.g., intents that are directed to device operation rather than specialized, complex medical procedures and nomenclature).

This optimization and prediction described in regard to FIGS. 5B and 5C is of particular importance in implementing an EMS digital assistant 104 on a smartphone. In a cloud computing environment (and to a lesser, but still substantial extent on an edge server), a large and non-specific general model can apply and execute an essentially unlimited number of sub-models to optimize confidence in the output at least because of large processing and memory capacity available on a cloud server. However, these resources are more limited on a smartphone as is the time available for natural language processing in the context of emergency care. Thus the improved efficiency and accuracy that evolves as the emergency care progresses as provided by the system in FIG. 5B improves both the functioning of the EMS digital assistant 104 and the care provided to the patient.

Additionally, these particular model selections may enable the EMS digital assistant 104 to provide specific, and possibly limited, options for unstructured text input (e.g., menu options and/or suggestions for speech options) at the user interface 504. This may tailor the unstructured text input to expected input and specific contexts. By guiding the caregiver in providing this type of input, the EMS digital assistant 104 described herein may further improve the efficiency and efficacy of care provided by the caregivers 106.

As explained above, in some examples a digital assistant (e.g., the EMS digital assistant 104 of FIG. 1) is configured to execute a dialogue process in which the digital assistant converses with a caregiver (e.g., the caregiver 106 of FIG. 1). FIG. 6 illustrates an example dialogue process 600 in accord with these examples.

As shown in FIG. 6, the process 600 starts with a user interface (e.g., the user interface 504 of FIG. 5A) receiving 602 input from the caregiver. This input is unstructured text obtained via one or more of a microphone, a keyboard, a touchscreen, computer vision (e.g., information obtained by the application of artificial intelligence and/or machine learning via the digital assistant 104 to a digital image or video), virtual reality, augmented reality, and/or information received via an internal or external application program interface (API). In some examples, the input may be, for example, tactile input in the form of keystrokes on a keyboard or touches on a touchscreen. Alternatively or additionally, the input may be speech. In response to receiving the input, the user interface derives input data from the input, generates a communication request including the input data, and passes the communication request to a channel handler (e.g., the channel handler 506 of FIG. 5A) for processing.

Continuing with the process 600, the channel handler determines 604 whether the input data is text data. For instance, in some examples, the channel handler identifies a type of channel (e.g., audio or tactile) from which the input was received. Alternatively or additionally, in some examples, the channel handler inspects the input data itself or references a flag set in the communication from the user interface to identify whether the input data is text data. Where the channel hander determines 604 that the input data is text data, the channel handler passes the input data to an NLP (e.g., the NLP 510 of FIG. 5A) for subsequent processing. Where the channel handler determines 604 that the input data is audio data, the channel handler passes the input data to an ASR engine (e.g., the ASR engine 508 of FIG. 5A) for subsequent processing.

Continuing with the process 600, the ASR engine converts 606 the input data to text data and passes the converted text to the NLP for subsequent processing. For instance, in some examples, the ASR engine executes an ASR process configured to recognize human language utterances within the input data and to render textual representations of the utterances. Next, the ASR engine passes the text rendered by the ASR process to the NLP for subsequent processing.

Continuing with the process 600, the NLP identifies 608, within the input text data, one or more intents and one or more values associated with each of the one or more intents. For instance, in some examples, the NLP identifies 608 intents and values by applying one or more natural language processing models trained to understand medical terminology, syntax, and grammar utilized by caregivers. These one or more models may include, for example, a general model (e.g., the general model 511 of FIG. 5B), one or more contextual models (e.g., the contextual models 550-555 of FIG. 5B), and/or one or more sub-contextual models (e.g., the sub-contextual models 560-569 of FIG. 5B).

Continuing with the process 600, the NLP identifies 610 one or more intent handlers (e.g., one or more of the intent handlers 512 of FIG. 5A) configured to fulfill the identified intents. For instance, in some examples, the NLP identifies 610 the one or more intent handlers by locating an association between the intents and the intent handlers within a data structure that associates intent identifiers with identifiers of intent handlers.

Continuing with the process 600, the NLP dispatches 612 each intent and its associated one or more values to its associated intent handler. For instance, in some examples, the NLP executes a function call to the intent handler with the one or more values as arguments. Processes executed by some example intent handlers in response to a function call are described further below with reference to FIGS. 7A-7D.

Continuing with the process 600, the NLP receives 614 output data from each intent handler that was dispatched an intent within the operation 612. For instance, in some examples, the NLP receives output data in response to each of the function calls executed in the operation 612. Next, the NLP passes each portion of output data to the channel handler for subsequent processing.

Continuing with the process 600, the channel handler converts 616 the output data to a type associated with an output channel. For instance, in some examples, the channel handler locates an input channel associated with the request corresponding to the output data and converts the output data to the type of the input channel. In certain examples, the channel hander locates the input channel by searching an associative data structure that relates requests with input channels. Next, the channel handler passes a response including the output data and an identifier of the output channel to the user interface.

Continuing with the process 600, the user interface renders 618 the output data via the output channel, thereby responding to the caregiver's request, and the process 600 returns to the operation 602.

Within implementations that include an edge server (e.g., the edge server 314 of FIG. 3B), the processes illustrated in FIG. 6 can be distributed across a first EMS digital assistant (e.g., the EMS digital assistant 104A of FIG. 3B) hosted by a first device and a second EMS digital assistant (e.g., the EMS digital assistant 104B of FIG. 3B) hosted by the edge server. For instance, in some examples, the first EMS digital assistant executes the operations 602 and 604, and the second EMS digital assistant executes the remaining operations. These examples advantageously leverage the edge server's ability to efficiently execute compute-intensive machine learning operations.

As explained above, in some examples a user interface navigator (e.g., the UI navigator 512A of FIG. 5A) is configured to fulfill intents to navigate a user interface of a charting application (e.g., the patient charting application 306A and/or the patient charting application 306B of FIG. 3A or 3B). FIG. 7A illustrates an example navigation process 700 executed by the user interface navigator in accord with these examples.

As shown in FIG. 7, the process 700 starts with the user interface navigator receiving 702 from an NLP (e.g., the NLP 510) an identifier of a slot and a value for the slot. The received slot identifier indicates that the received slot value identifies one or more user interface controls displayable by the charting application and to which the caregiver wishes to navigate. For instance, in some examples, the slot value is a name of an ePCR section, category, sub-category, page, or field.

Continuing with the process 700, the user interface navigator identifies 704 an API call implemented by the charting application to cause the charting application to display a screen that includes the one or more user interface controls, or where no externally invocable screen includes the one or more user controls, an invocable screen nearest the one or more controls within the user interface graph implemented by the charting application. For instance, in some examples, the user interface navigator identifies 704 the API call by locating an association between the one or more user interface controls and the API call within an associative data structure that maps slot values to API calls.

Continuing with the process 700, the user interface navigator transmits 706 a navigation request to the charting application. For instance, in some examples, the user interface navigator executes the API call identified in the operation 704. Also within the operation 706, the user interface navigator receives a response from the API call that indicates whether the API call was successfully processed.

Continuing with the process 700, the user interface navigator constructs 708 output text based on the response to the navigation request. For instance, in some examples, the user interface navigator constructs 708 the output text as a textual human language communication that indicates whether the API call was successfully processed. Next, the user interface navigator returns 710 the output text to the NLP, and the process 700 ends.

As explained above, in some examples a data recorder (e.g., the data recorder 512B of FIG. 5A) is configured to fulfill intents to record ePCR data in manner compliant with the schema, reporting format, and/or content standard associated with the ePCR. FIG. 7B illustrates an example ePCR data recordation process 720 executed by the data recorder in accord with these examples.

As shown in FIG. 7, the process 720 starts with the data recorder receiving 722, from an NLP (e.g., the NLP 510), one or more identifiers of one or more slots paired with one or more values for the one or more slots. The one or more slot identifiers indicate one or more standard ePCR data elements for which the paired values are to be recorded. For instance, in one example, the slot identifiers indicate standard elements such as blood pressure, heart rate, pulse oxygen, and respiratory rate. The one or more slot values indicate values to be recorded for the standard elements. In this example, the slot values include strings such as “120/80”, “72”, “98”, and “18”. In some examples, one of the one or more slot values can further indicate information such as a time associated with the ePCR data to be recorded and the source of the ePCR data as reported by the caregiver.

Continuing with the process 720, the data recorder maps 724 each of the slot values to one or more transformations required to put the slot value in compliance with a standard element associated with the slot value. For instance, in some examples, the data recorder maps 724 each slot value to one or more transformations by locating, within a data structure that associates slot identifiers with transformations, an association between the one or more transformations and the slot identifier paired with the slot value. To illustrate, consider an ePCR documentation standard that requires systolic blood pressure measurements be recorded separately from diastolic pressure measurements and that further requires that each type of blood pressure measurement be recorded as a numeric value. Given these storage requirements, in the operation 724, the data recorder maps a slot value “120/80” paired with a blood pressure slot identifier to a deconstruction transformation and a data type transformation associated with the blood pressure slot identifier.

Continuing with the process 720, the data recorder transforms 726 each of the slot values via the transformations to which the slot is mapped. The examples described herein support an arbitrary number and type of transformations. Some example transformations include deconstructing a slot value to produce two or more sub-values; combining slot values to generate super-values; changing the data type of slot values, sub-values, or super-values; augmenting slot values, sub-values, or super-values with static or dynamic values; and re-encoding values to change symbol sets, to name a few. For example, where transforming a slot value encoding a blood pressure measurement, the data recorder may parse the slot value “120/80” into sub-values of “120” and “80”. Further, in this example, the data recorder may convert the string “120” to a numeric value of 120 and the string “80” to a numeric value of 80. In another example, the data recorder may re-encode a string “today” to a timestamp value for the current day when transforming a slot value paired to a standard element dealing with time.

Continuing with the process 720, the data recorder validates 728 the transformed values to ensure the transformed values meet all validation requirements. For instance, in some examples, the data recorder validates 728 the transformed values by comparing each transformed value to a set of valid values associated with its mapped standard element to ensure that the transformed value falls within the set of valid values. These sets of valid values can be enumerated values or expressed, for example, as one or more regular expressions. In these examples, the data recorder identifies the set of valid values to use for comparison purposes by locating an association between the mapped standard element and the set of valid values within a data structure that associates standard elements and sets of valid values. For instance, in one example, the data recorder may compare the number value of 120 to a range of valid systolic blood pressure values. As another example, the data recorder may compare a date of birth of a patient to the current date to ensure that the date meets an applicable validation rule (e.g., is not a future date) and to ensure it meets an applicable validation format (e.g., YY-MM-DD).

Continuing with the process 720, the data recorder stores 730 an association between the mapped standard element and the validated value in, for example, a data structure that associates standard elements with validated values. For instance, the data recorder may associate the value 120 with the systolic blood pressure standard element and the value 80 with the diastolic blood pressure standard element. It should be noted that, in some instances of the operation 730, the data recorder may determine that a validated value already exists for a mapped standard element at given time. In this situation, the data recorder stores 730 an association between the mapped standard element and the validated value coming from a source with the highest authority (e.g., a device or system of record).

Continuing with the process 720, the data recorder constructs 732 output text based on the results of preceding operations of the process 720. For instance, in some examples, the data recorder constructs 708 the output text as a textual human language communication that indicates whether the slot values were successfully mapped, transformed, and validated. For example, the output text may include “Added: Vital Signs.” In addition, depending on the mode of operation of the EMS digital assistant executing the data recorder, the data recorder may construct 708 further output text that prompts the caregiver to input ePCR data that is procedurally related to the standard elements associated with validated values in the operation 730. In some examples, the data recorder identifies ePCR data that is procedurally related to these standard elements by locating, within a data structure that associates standard element identifiers with procedurally related standard element identifiers. Alternatively or additionally, in some examples, the data recorder identifies ePCR data that is procedurally related to the standard elements by applying a machine learning model, such as the caregiver activity sequence model 590 of FIG. 5B, to the slot identifiers and slot values received in operation 722. Additionally or alternatively, where an incomplete portion of ePCR data was recorded, the data recorder may (depending on the current operational mode) construct 708 output text that prompts the user to input the additional data required to complete the ePCR data, as illustrated above in Table 6. It should be noted that, in some instances of the operation 732, the data recorder may construct 708 output text that prompts the user to specify the source of ePCR data where a validated value already exists for a mapped standard element at given time. Next, the data recorder returns 734 the output text to the NLP, and the process 720 ends.

As explained above, in some examples an image capturer (e.g., the image capturer 512C of FIG. 5A) is configured to fulfill intents to capture images using a camera incorporated within the computing device hosting the image capturer. FIG. 7C illustrates an example image capture process 740 executed by the image capturer in accord with these examples.

As shown in FIG. 7, the process 740 starts with the image capturer receiving 742 from an NLP (e.g., the NLP 510) an identifier of a slot and a value for the slot. The received slot identifier indicates a camera within the host device targeted for control by the intent. Examples of cameras that can be indicated via the slot identifier include a front camera or a back camera, among others. The received slot value indicates a command to issue to the camera identified by the slot identifier. Examples of commands that can be indicated via the slot value include a capture image command and a capture movie command, among others.

Continuing with the process 740, the image capturer executes 744 the command indicated by the slot value. For instance, in some examples, the image capturer executes one or more operating system API calls to control image capture via the targeted camera (e.g., an image of the medication 120 of FIG. 1). In these examples, the image capturer stores the captured image in memory for subsequent processing.

Continuing with the process 740, the image capturer scans 746 captured images for symbols relevant to one or more ePCR data fields (e.g., barcodes, QR codes, and/or typed or handwritten text). For instance, in some examples, the image capturer processes images using any of a variety of commercially available barcode scanning and/or optical character recognition processes. In certain examples, within the operation 746, the image capturer highlights symbols recognized within the images and displays the images with the highlights via a display of the computing device hosting the image capturer. In these examples, the image capturer also stores ePCR data derived from the recognized symbols in association with the images.

Continuing with the process 740, the image capturer constructs 748 output text based on the results of the operation 744. For instance, in some examples, the image capturer constructs 748 the output text as a textual human language communication that indicates whether the command was successfully executed. Next, the image capturer returns 750 the output text to the NLP, and the process 740 ends.

As explained above, in some examples a data reporter (e.g., the data reporter 512D of FIG. 5A) is configured to fulfill intents to report recorded ePCR data values. FIG. 7D illustrates an example data reporting process 760 executed by the data reporter in accord with these examples.

As shown in FIG. 7, the process 760 starts with the data reporter receiving 762 from an NLP (e.g., the NLP 510) one or more identifiers of one or more slots paired with one or more values for the one or more slots. In some examples, one or more of the received slot values indicates one or more elements of ePCR data targeted for reporting by the intent. Examples of the ePCR data targeted for reporting can include an ePCR section, category, sub-category, page, or field. Moreover, in some examples, one or more of the received slot values indicates a point in time for which a value of the ePCR data is requested.

Continuing with the process 760, the data reporter retrieves 764 the requested ePCR data value. For instance, the data reporter may access a local ePCR data store (e.g., the ePCR data store 312A of FIG. 3A or 3B) or a remote data store (e.g., the ePCR data store 312B of FIG. 3A or 3B or the ePCR data store 312C of FIG. 3B) to retrieve the requested ePCR data value.

Continuing with the process 760, the data reporter constructs 766 output text based on the results of the operation 764. For instance, in some examples, the data reporter constructs 766 the output text as a textual human language communication that indicates the requested ePCR data value. Next, the data reporter returns 768 the output text to the NLP, and the process 760 ends.

It should be noted that, in some examples, the data reporter may receive one or more slot values that indicate an intent to report ePCR data at one or more points of time in the future. In these examples, the data reporter configures a timer to repeatedly call the data reporter at the future points in time specified by the one or more slot values.

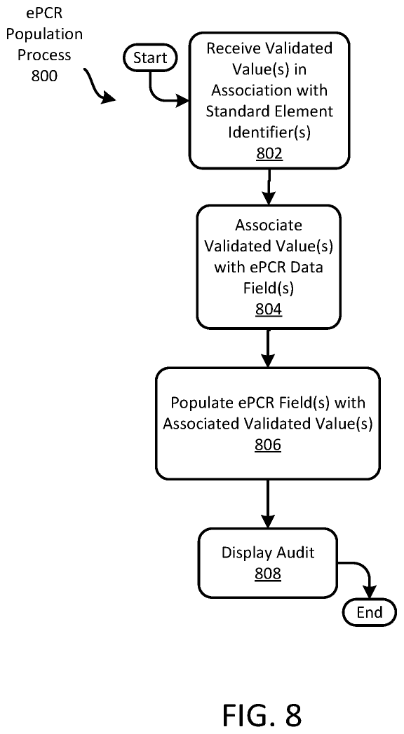

As explained above, in some examples a digital assistant (e.g., the EMS digital assistant 304 of FIG. 3A or 3B) is configured to populate data fields within an ePCR with previously recorded ePCR data. FIG. 8 illustrates an example population process 800 executed by the digital assistant in accord with these examples.

As shown in FIG. 8, the process 800 starts with the digital assistant receiving 802 validated ePCR data values in association with ePCR standard data element identifiers. In some examples, this validated ePCR data is previously stored by an ePCR data recorder (e.g., the data recorder 512B of FIG. 5A) in a storage operation (e.g., the operation 730 of FIG. 7B) executed during a data recordation process (e.g., the data recordation process 720 of FIG. 7B). In some examples, the digital assistant receives 802 the validated ePCR data from an EMS digital assistant (e.g., the EMS digital assistant 104 of FIG. 3A or 3B). For instance, in certain examples, the digital assistant requests the validated ePCR data from the EMS digital assistant via an API call. The digital assistant may make this API call in response to scanning a QR code generated by the EMS digital assistant as described above with reference to FIG. 2I.

Continuing with the process 800, the digital assistant associates 804 the validated ePCR data values received in the operation 802 with ePCR data fields to be populated by the validated ePCR data values. These ePCR data fields may be part of an ePCR accessible via a charting application (e.g., the patient charting application 306A and/or the patient charting application 306B of FIG. 3A or 3B). As such, the ePCR data fields may reside in a data store local to the digital assistant (e.g., the ePCR data store 312A of FIG. 3A or 3B) or a data store remote from the digital assistant (e.g., the ePCR data store 312B of FIG. 3A or 3B or the ePCR data store 312 of FIG. 3B).

In some examples, the digital assistant maps 804 the validated ePCR data values to the ePCR data fields via their common association with standard elements identifiers. In these examples, the digital assistant locates, within a data structure that associates standard element identifiers with ePCR data fields, each association involving a standard element identifier received in the operation 802. Next, in these examples, the digital assistant maps 804 the ePCR data field in each association to the ePCR data value associated with the standard element identifier in the association.

Next, the digital assistant populates 806 the ePCR data fields with the validated ePCR data values paired with the standard elements associated with the ePCR data fields. For instance, in some examples, the digital assistant stores the validated ePCR value in the ePCR data field.

Continuing with the process 800, the digital assistant displays 808 an audit trial that lists the ePCR data fields populated within the operation 806, and the process 800 ends.

Turning now to FIG. 9, a data flow diagram of a training system 900 is shown. The training system 900 processes a variety of data to train one or more natural language processors as described herein. For example, the data may be from sources including, but not limited to, an ePCR standard, historical ePCR records, publicly available historical NEMSIS records, historical dispatch records, historical billing account records, historical billing claims or 837 EDI data, historical payer explanations of benefits or 835 EDI data, X12 Healthcare EDI standard, a medical device, shorthand terminology, a user specific vocabulary, report definitions, Standard Query Language (SQL) examples, HL7 version 2, version 3, CDA and FHIR standards, SNOMED CT clinical terminology, HCPCS and CPT procedure standards, internationalized and localized versions of all of the above, and combinations thereof.

As shown in FIG. 9, the system 900 includes a vocabulary extractor 904, a natural language generator 906, a NLP trainer 910, and the NLP 510 of FIG. 5A. The system 900 also includes a medical documentation standard data store 902A, a standards of care data store 902B, a treatment protocol data store 902C, an observed order of population data store 902D, an encounter histories data store 902E, and a training and testing data store 908. In some examples, the system 900 is implemented using a server environment, such as the server environment 310 of FIG. 3A or 3B, although implementation via less powerful computing devices is possible. For instance, in at least one example, the system 900 is implemented using an edge server (e.g., the edge server 314 of FIG. 3B).

Each of the data stores 902A-902E are curated sources of structured text data that can be used to build training and testing data housed within the training and testing data store 908. This training and testing data specifies natural language communications that use the medical terminology, syntax, and grammar of caregivers. In some examples, the documentation standard data store 902A includes structure text derived from the schema, reporting format, and/or content standard associated with the ePCR. The standards of care data store 902B includes structured text derived from formal guidelines that are generally accepted in the medical community for the treatment of a disease or condition. The treatment protocol data store 902C includes structured text derived from policies established by a particular medical organization (e.g., the organization for which the NLP 510 is being trained). The observed order of population 902D includes structured text that specifies the order in which ePCRs data fields were completed in actual patient encounters. The data store 902E stores unstructured textual renderings of human language communications uttered during actual patient encounters.

Continuing with the system 900, the vocabulary extractor 904 is configured to retrieve and process structured text data from each of the data stores 902A-902E to extract slots and slot values from the text data. In these examples, the vocabulary extractor 904 maintains a list of formats utilized by each of the data stores 902A-902E and processes text data retrieved from each data store using its associated format. In this way, the vocabulary extractor 904 can consistently extract slots and slot values from the text data retrieved from each of the data stores 902A-902E.

Continuing with the system 900, the human language generator 906 is configured to receive slots and slot values from the vocabulary extractor 904 and generate human language communications using a variety of slots and slot values. For example, where vocabulary extractor 904 passes a blood pressure slots having a value of 120/80, the human language generator 906 may construct a sentence such as, “The patient's blood pressure is 120/80.” Next, the human language generator annotates each of the generated human language communications with labels indicating its associated intent, slot(s), and slot value(s) and stores these annotated communications in the data store 908 for subsequent processing.

Continuing with the system 900, the natural language processor trainer 910 is configured to train one or more NLP models that make up the trained NLP 510. In some examples, the trainer 910 retrieves a portion of the annotated human language communications from the data store 908 and trains one or more NLP models by executing a training process (e.g., stochastic gradient descent, transfer learning based on a previously trained model, etc.) using the retrieve data. In some examples, the NLP models may be models based on a data science and machine learning framework, such as, but not limited to, TensorFlow, Brain, Keras, Apache MXNET, etc. Once the models are trained, the natural language processor trainer 910 tests the trained models to determine accuracy. Where the accuracy transgresses a required threshold, the trainer 910 publishes the models, which become a trained NLP for production use (e.g., as the trained NLP 510).

FIG. 10A illustrates an example of a logical and physical architecture of an EMS digital assistant as part of a SaaS platform. In an implementation, the EMS digital assistant 104 executing on the mobile device 102, for example, a smartphone in a mobile EMS environment 1004, may communicatively couple to a charting system server 1018. The EMS digital assistant 104 interoperate with a positioning system 1040 included in the mobile device 102. The positioning system 1040 may use global positioning system (e.g., satellite positioning) and/or cellular positioning data to locate the mobile device 102. The EMS digital assistant 104 may use the positioning data to determine a context for the mobile device 102 and this determined context may enable the EMS digital assistant 104 to select and adapt the model selection as described in regard to FIGS. 5B and 5C. The mobile EMS environment 1004 may further include one or more medical device(s) 1032. In various implementations, the medical device(s) 1032 can include a patient treatment device, or another kind of device that includes patient monitoring and/or patient treatment capabilities, according to examples of the present disclosure. For example, the medical device(s) 1032 include a defibrillator and can be configured to deliver therapeutic electric shocks to the patient. In some examples, the medical device(s) 1032 can deliver other types of treatments, such as ventilation, operating a respirator, and/or administering drugs or other medication.

In such an implementation, the EMS digital assistant 104 may receive and utilize data from other elements of the SaaS platform 1026 executing in a cloud environment 1002. The platform 1026 may include a CAD system server 1030, a navigation system server 1028, a patient charting system server 1022, a medical billing system server 1067, a medical device case data store 1024, and a charting system data store 1020. The mobile EMS environment 1004 may also include an emergency vehicle, such as an ambulance, a fire engine, an EMS crew transport vehicle, and/or a helicopter. In an implementation, the SaaS platform 1026 enables sharing of information between entities of the platform and enables the EMS digital assistant 104 to enhance patient care through advanced caregiver guidance and recordation based on this sharing. For example, initiation of a call by the CAD 1030 and communication to the EMS digital assistant 104 of the initiated call may enable the EMS digital assistant 104 to query a medical record repository 1005. The EMS digital assistant 104 may store query results in the ePCR and/or generate caregiver prompts based on the query result. Further, the EMS digital assistant may provide query results to the charting system server 1018 and/or the billing system server 1067. Alternatively, the CAD 1030 may communicate with the charting system 1018 and the charting system 1018 may then communicate with the medical record repository 1005 and provide query results to the EMS digital assistant 104. The medical billing system 1067 may receive and/or provide charting information and/or patient care information (e.g., based on a medical history provided by billing records) to the EMS digital assistant 104 during or after the medical event via communications with the charting system server 1018.

As shown in FIG. 10A, the cloud environment 1002 may be implemented within a data center or other high capacity computing facility with high speed internet connectivity. For instance, the cloud environment 1002 can be implemented via a commercially available cloud computing service, such as MICROSOFT AZURE or AMAZON WEB SERVICES. The platform 1026 may include a plurality of dedicated servers (e.g., a farm or cluster of computer systems) within the data center that are interconnected via a high speed, private network. Each of the servers illustrated within the platform 1026 may be one or more physical and/or one or more virtual servers. The servers can include one or more application servers, web servers, and/or data base servers. The servers can include enterprise servers configured to support an organization as a single tenant and/or cloud servers configured to support multiple organizations as multiple tenants.

It should be noted that the software applications hosted by servers within the platform 1026 are configured to expose application programming interfaces (APIs) that enable the software applications to communicate with one another. These APIs are configured to receive, process, and respond to commands issued by software applications hosted on the same server or a different server in the platform. For instance, these APIs enable any of the servers in the platform 1026 to transmit queries, information, patient reference codes etc. and otherwise communicate with one or more other servers in the platform 1026 and/or with the EMS digital assistant 104.

The APIs may be implemented using a variety of interoperability standards and architectural styles. For instance, in one example, the APIs are web services interfaces implemented using a representational state transfer (REST) architectural style. In this example, the APIs communicate with a client process using Hypertext Transfer Protocol (HTTP) along with JavaScript Object Notation and/or extensible markup language. In some examples, portions of the HTTP communications can be encrypted to increase security. Alternatively or additionally, in some examples, the APIs are implemented as a .NET web API that responds to HTTP posts to particular uniform resource locators. Alternatively or additionally, in some examples, the APIs are implemented using simple file transfer protocol commands and/or a proprietary application protocol accessible via a transmission control protocol socket. Thus, the APIs described herein are not limited to a particular implementation.

The network within the cloud environment 1002 and the local network with the mobile EMS environment 1004 can include one or more communication networks through which the computing devices within these environments send, receive, and/or exchange data. In various implementations, the network can include a cellular communication network and/or a computer network. In some examples, the network includes and supports wireless network and/or wired connections. For instance, in these examples, the network may support one or more networking standards including PAN standards, such as universal serial bus (USB), BLUETOOTH, controller area network (CAN), or ZIGBEE; one or more LAN standards, such as Wireless Ethernet, Ethernet, and transfer control protocol/internet protocol (TCP/IP); and one of more WAN standards, such as TCP/IP, GSM, and CMDA, among others. As such, the network may include both private networks, such as local area networks, and public networks, such as the Internet. It should be noted that, in some examples, the network may include one or more intermediate devices involved in the routing of packets from one endpoint to another. However, in other examples, the network can involve only two endpoints that each have a network connection directly with the other.

The data store 1020 may be implemented by, for example, a database (e.g., a relational database) and stored on a non-transitory storage medium. The data store 1020 is configured to store ePCRs generated by the EMS digital assistant 104.

In some examples, the charting system server 1018 is configured to interoperate with the CAD system server 1030, the navigation system server 1028, the billing system server 1067, and/or the case data store 1024 to acquire patient identification data and/or medical records for patients. It should be noted that, in some examples, the charting system server 1018 is configured to periodically update medical records by interoperating with the other servers in the platform 1026 and/or devices within the mobile EMS environment 1004. For instance, in one example, the charting system server 1018 periodically requests updated billing codes from the billing system server 1067 and updates medical records stored in the data store 1020 accordingly. These billing codes are a source of information for previous medical treatments. For instance, billing codes can indicate that a patient received treatment for asthma, treatment for cardiac arrest, treatment for a drug overdose, prescription information, and/or recent surgeries. This information may be clinically actionable and relevant. For example, stitches from recent surgeries could reopen. Devices implanted during surgery may need to be addressed. Treatments for drug overdose may indicate a need to avoid opioids. Repeated treatments and prescriptions could indicate chronic conditions and/or contraindications.

The CAD system server 1030 may receive requests to record calls from a public safety answering point and process the requests to generate and store call records. The CAD system server 1030 may transmit dispatch requests to an EMS agency to dispatch EMS personnel (e.g., the care provider 106 of FIGS. 1A-1C) to service calls. The CAD system server 1030 may transmit addresses to call locations to the EMS digital assistant 104 so that the EMS digital assistant 104 can acquire routes to call locations by interoperating with the positioning system 1040 and/or the navigation system server 1028. In an implementation, the EMS digital assistant 104 may provide real time, step by step directions to call locations via the routes.

The case data store 1024 receives case files uploaded by the medical devices 1032. The case data store 1024 can be implemented by, for example, a database (e.g., a relational database) and stored on a non-transitory storage medium. In an implementation, the case data store 1024 includes a plurality of records that store case data derived from case files from a plurality of medical devices used to treat patients during encounters. Moreover, in some examples, the case data store 1024 can store complete copies of the case files themselves (e.g., as large binary objects). The case data stored in the case data store 1024 can document patient encounters from the point of view of medical devices. As such, case data generated by a medical device during a patient encounter can include an identifier of the medical device, physiologic parameter values of the patient recorded by the medical device during the encounter, characteristics of treatment provided by the medical device to a patient during the encounter, actions taken by care providers during the encounter, and timestamps associated with medical device case data. For instance, where the medical device is a defibrillator, the case data can include patient physiological parameters such as ECG data for the patient, as well as characteristics of therapeutic shocks delivered by the defibrillator to the patient, CPR performance data, and timestamps reflecting a power-on time for the defibrillator and associated with recorded case data, among other information. The EMS digital assistant 104 may receive case data from the medical device(s) 1032 via the charting system server 1018 and/or via short-range communications with the medical device(s) 1032.

The data stores 1020 and 1024 can be organized according to a variety of physical and/or logical structures. In at least one example, the data stores 1020 and 1024 are implemented within a relational database having a highly normalized schema and accessible via a structured query language (SQL) engine, such as ORACLE or SQL-SERVER. This schema can, in some implementations, include columns and data that enable the data stores 1020 and 1024 to house data for multiple tenants. In addition, although the description provided above illustrates the data stores 1020 and 1024 as relational databases, the examples described herein are not limited to that particular physical form. Other databases may include flat files maintained by an operating system and including serialized, proprietary data structures, hierarchical database, xml files, NoSQL databases, document-oriented databases and the like. Thus, the data stores 1020 and 1024 as described herein are not limited to a particular implementation.

Continuing with FIG. 10A, the billing system server 1067 implements a medical billing system. The billing system server 1067 can store patient identification data, information regarding claims involving patients, payments status of the claims, and the like. The patient identification data stored in the billing system server 1067 can include, for example, patient provider and insurance information.

Interoperations between the EMS digital assistant 104 and the various elements of the SaaS platform 1026 may enable the EMS digital assistant 104 to provide various types of information relevant to the patient care and the EMS interoperation as shown in Table 7. The information in Table 7 is exemplary and not limiting of the disclosure. These examples are of unstructured queries from the caregiver that the EMS digital assistant 104 may recognize and respond to via API interoperations with one or more of the CAD system server 1030, the navigation system server 1028, the billing system server 1067, the charting system server 1018, the medical record repository 1005, the charting data store 1020, and the case data store 1024. The API interoperations with the billing system server 1067, the medical record repository 1005, the charting data store 1020, and the case data store 1024 may occur via the charting system server 1018. As one example of a query, the caregiver may ask “Have we transported this patient before?” In response, the EMS digital assistant 104 may access the platform 1026 and provide previous transport information audibly and/or visibly for the caregiver. Similarly, the other examples in Table 7 may be formulated as a question from the caregiver. Additionally or alternatively, in an implementation, the EMS digital assistant 104 may initiate a query to the platform and provide prompts or other caregiver guidance that provides the exemplary information without an initiating query from the caregiver 106. For example, based on context and/or procedural relationships between data fields and/or caregiver activities, the EMS digital assistant 104 may automatically obtain and provide the information in the examples of Table 7. As one example, the EMS digital assistant 104 may initiate a query regarding previous transports based on information provided to the ePCR (e.g., patient demographics) and automatically inform the caregiver “Agency X previously transported this patient to Hospital J for drug overdose on Mar. 10, 2021.” The EMS digital assistant 104 may further ask the caregiver to request any further information based on that information automatically provided. For example, “Would you like me to identify a preferred provider and any contraindications based on the previous transport?”

TABLE 7

| Category | Examples |
|---|---|
| Historical Patient Care | Previous transports of patient by agency |
| | Previous risks and/or complications for |
| | the patient |
| | Progressive information as patient call |
| | evolves |
| Predictive patient | Current level of service |
| care analysis | Level of care data points |
| | ICD coding suggestions based on |
| | assessments, protocols, medications, |
| | airway, and/or procedures |
| Patient care reimbursement | Self-pay patient |
| analysis | Confirmation of health insurance |
| | Preferred providers |
| | Necessary pre-authorizations |
| Patient care knowledge base | Current medication interactions |
| | Passive protocols for patient |
| | Active protocols for patient |
| | Next step in a protocol (e.g., trauma, |
| | sepsis, cardiac, respiratory, etc.) |
| Patient Coordination of | Coordinate care with destination |
| Care with Dispatch | Send current patient report to hospital |
| | while patient is en route |
| | Send historical patient report to hospital |
| Dispatch and Navigation | Find closest medical facility for medical |
| | care needed by patient based on provided |
| | EMS care |
| | Find best location for transport vehicle at |
| | the emergency scene |
| | Schedule medical procedure and transport |
| | for a future date (e.g., record and provide |
| | basic information and specialty care |
| | transport interactive dialog) |
| Data Exchange | Previous patient hospital outcomes |
| and analytics | Crew level of service and vehicle |
| | allocation for particular time period |
| | Analysis of destinations filtered by patient |
| | care requirements |
| Billing | Open an account, provide insurance |
| | information, confirm patient |
| | demographics, claims checklist, medical |
| | necessity analysis, closest facility |
| | analysis, insurance eligibility analysis, |
| | billing worklists, billing rules |

In combination, the systems illustrated in FIG. 10A can produce accurate and comprehensive documentation that improves continuity of patient care and overall patient health outcomes. More specifically, continuity of care may benefit from a record that thoroughly describes symptoms, physiological metrics, and treatments provided.

FIG. 10B illustrates an example of a logical and physical architecture of an EMS digital assistant as part of a SaaS platform 1027. As shown in FIG. 10B, the platform 1027 includes many of the features of the platform 1026 of FIG. 10A. The platform 1027 further includes an edge server 314. In the platform 1027, the mobile device 102 hosts an EMS digital assistant 104A, and the edge server 314 hosts an EMS digital assistant 104B.

In an implementation, the EMS digital assistant 104A executing on the mobile computing device 102 (e.g., a smartphone in a mobile EMS environment 1004) and/or the EMS digital assistant 104B executing on the edge server 314 may communicatively couple to a charting system server 1018. The EMS digital assistant 104A may interoperate with a positioning system 1040 included in the mobile device 102. The positioning system 1040 may use global positioning system (e.g., satellite positioning) and/or cellular positioning data to locate the mobile device 102. The EMS digital assistant 104A may use the positioning data to determine a context for the mobile device 102 and this determined context may enable the EMS digital assistant 104A and/or the EMS digital assistant 104B to select and adapt the model selection as described in regard to FIGS. 5B and 5C. The mobile EMS environment 1004 may further include one or more medical device(s) 1032 and the edge server 314. Although the edge server 314 is illustrated as a distinct device in FIG. 10B, in some examples the edge server 314 is incorporated into one of the medical device(s) 1032.

In an implementation, the EMS digital assistants 104A and/or 104B may receive and utilize data from other elements of the platform 1027 executing in a cloud environment 1002. The platform 1027 may include the CAD system server 1030, the navigation system server 1028, the patient charting system server 1022, the medical billing system server 1067, the medical device case data store 1024, and the charting system data store 1020 of FIG. 10A. In an implementation, the SaaS platform 1027 enables sharing of information between entities of the platform and enables the EMS digital assistants 104A and/or 104B to enhance patient care through advanced caregiver guidance and recordation based on this sharing. For example, initiation of a call by the CAD 1030 and communication to the EMS digital assistants 104A and/or 104B of the initiated call may enable the EMS digital assistants 104A and/or 104B to query a medical record repository 1005. The EMS digital assistants 104A and/or 104B may store query results in the ePCR and/or generate caregiver prompts based on the query result. Further, the EMS digital assistants 104A and/or 104B may provide query results to the charting system server 1018 and/or the billing system server 1067. Alternatively, the CAD 1030 may communicate with the charting system 1018 and the charting system 1018 may then communicate with the medical record repository 1005 and provide query results to the EMS digital assistants 104A and/or 104B. The medical billing system 1067 may receive and/or provide charting information and/or patient care information (e.g., based on a medical history provided by billing records) to the EMS digital assistants 104A and/or 104B during or after the medical event via communications with the charting system server 1018.

It should be noted that the software applications hosted by servers within the platform 1027 are configured to expose application programming interfaces (APIs) that enable the software applications to communicate with one another. These APIs are configured to receive, process, and respond to commands issued by software applications hosted on the same server or a different server in the platform. For instance, these APIs enable any of the servers in the platform 1027 to transmit queries, information, patient reference codes etc. and otherwise communicate with one or more other servers in the platform 1027 and/or with the EMS digital assistants 104A and/or 104B.

Interoperations between the EMS digital assistants 104A and/or 104B and the various elements of the SaaS platform 1027 may enable the EMS digital assistants 104A and/or 104B to provide various types of information relevant to the patient care and the EMS interaction. For instance, in some examples, the CAD system server 1030 may transmit addresses to call locations to the EMS digital assistants 104A and/or 104B so that the EMS digital assistants 104A and/or 104B can acquire routes to call locations by interoperating with the positioning system 1040 and/or the navigation system server 1028. In an implementation, the EMS digital assistants 104A and/or 104B may provide real time, step by step directions to call locations via the routes. The EMS digital assistants 104A and/or 104B may receive case data from the medical device(s) 1032 via the charting system server 1018 and/or via short-range communications with the medical device(s) 1032. The data store 1020 is configured, in some examples to store ePCRs generated by the EMS digital assistants 104A and/or 104B. Table 7, which is provided above, lists additional types of information relevant to patient care that the EMS digital assistants 104A and/or 104B may access via one or more API calls.

In combination, the systems illustrated in FIG. 10B can produce accurate and comprehensive documentation that improves continuity of patient care and overall patient health outcomes. More specifically, continuity of care may benefit from a record that thoroughly describes symptoms, physiological metrics, and treatments provided.

The physical processors described herein are physical processors (i.e., an integrated circuit configured to execute operations on a respective device as specified by software and/or firmware stored in a computer storage medium) operably coupled, respectively, to at least one memory device. The processors may be intelligent hardware devices (for example, but not limited to, a central processing unit (CPU), a graphics processing unit (GPU), one or more microprocessors, a controller or microcontroller, an application specific integrated circuit (ASIC), a digital signal processor (DSP), etc.) designed to perform the functions described herein and operable to carry out instructions on a respective device. Each of the processors may be one or more processors and may be implemented as a combination of hardware devices (e.g., a combination of DSP and a microprocessor, a plurality of microprocessors, one or more microprocessors in conjunction with a DSP core, or another such configuration). Each of the processors may include multiple separate physical entities that may be distributed in an associated computing device. Each of the processors is configured to execute processor-readable, processor-executable software code containing one or more instructions or code for controlling the processors to perform the functions as described herein. The processors may utilize various architectures including but not limited to a complex instruction set computer (CISC) processor, a reduced instruction set computer (RISC) processor, or a minimal instruction set computer (MISC). In various implementations, each processor may be a single-threaded or a multi-threaded processor. The processors may be, for example, but not limited to, an Intel® Itanium® or Itanium 2® processor(s), AMD® Opteron®, Athlon MP® processor(s), a Motorola® line of processor, or an ARM, Intel Pentium Mobile, Intel Core i5 Mobile, AMD A6 Series, AMD Phenom II Quad Core Mobile, or like devices.

The memories refer generally to a computer storage medium, including but not limited to RAM, ROM, FLASH, disc drives, fuse devices, and portable storage media, such as Universal Serial Bus (USB) flash drives, etc. Each of the memories may include, for example, random access memory (RAM), or another dynamic storage device(s) and may include read only memory (ROM) or another static storage device(s) such as programmable read only memory (PROM) chips for storing static information such as instructions for a coupled processor. Each memory may include USB flash drives that may store operating systems and other applications. The USB flash drives may include input/output components, such as a wireless transmitter and/or USB connector that can be inserted into a USB port of another computing device. Each memory may be long term and/or short term are not to be limited to a particular type of memory or number of memories, or type of media upon which memory is stored. Each memory includes a non-transitory processor-readable storage medium (or media) that stores the processor-readable, processor-executable software code. Each memory may store information and instructions. For example, each memory may include flash memory and/or another storage media may be used, including removable or dedicated memory in a mobile or portable device. As another example, hard disks such as the Adaptec® family of SCSI drives, an optical disc, an array of disks such as RAID (e.g. the Adaptec family of RAID drives), or another mass storage devices may be used. Each memory may include removable storage media such as, for example, external hard-drives, floppy drives, flash drives, zip drives, compact disc-read only memory (CD-ROM), compact disc-re-writable (CD-RW), or digital video disk-read only memory (DVD-ROM).

Communicatively coupled devices as described herein may transmit and/or receive information via a wired and/or wireless communicative coupling. The information may include information stored in at least one memory. The information may include, for example, but not limited to, resuscitative treatment information, physiological information, patient information, rescuer and/or caregiver information, location information, rescue and/or medical treatment center information, etc. The communicative couplings may enable short-range and/or long-range wireless communication capabilities which may include communication via near field communication, ZIGBEE, WIFI, BLUETOOTH, satellite(s), radio waves, a computer network (e.g., the Internet), a cellular network, a LAN, WAN, a mesh network, an ad hoc network, or another network. The communicative couplings may include, for example, an RS-232 port for use with a modem-based dialup connection, a copper or fiber 10/100/1000 Ethernet port, or a BLUETOOTH or WIFI interface.

Displays as described herein may provide a graphical user interface (GUI). A particular display may be, for example, but not limited to, a touchscreen display, an augmented reality display/visor, a liquid crystal display (LCD), and/or a light emitting diode (LED) display. The touchscreen may be, for example, a pressure sensitive touchscreen or a capacitive touchscreen. The touchscreen may capture user input provided via touchscreen gestures and/or provided via exertions of pressure on a particular area of the screen. The displays may provide visual representations of data captured by and/or received at the medical device 170. The visual representations may include still images and/or video images (e.g., animated images).

The computing devices referred to herein may include one or more user input devices such as, for example, a keyboard, a mouse, joystick, trackball, or other pointing device, a microphone, a camera, etc. In an implementation, the user input devices may be configured to capture information, such as, for example, patient medical history (e.g., medical record information including age, gender, weight, body mass index, family history of heart disease, cardiac diagnosis, co-morbidity, medications, previous medical treatments, and/or other physiological information), physical examination results, patient identification, caregiver identification, healthcare facility information, etc.

The processor, memory, communication interfaces, input and/or output devices and other components described above are meant to exemplify some types of possibilities. In no way should the aforementioned examples limit the scope of the disclosure, as they are only exemplary embodiments of these components.

Various modifications and additions can be made to the exemplary embodiments discussed without departing from the scope of the present disclosure. For example, while the embodiments described above refer to particular features, the scope of the disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

It should be noted that the digital assistants described herein can be used in medical settings other than EMS. For instance, some examples can be useful in hospital, clinic, military medical treatment, home, and other non-EMS settings. It should also be noted that EMS care can include both emergency care (e.g., car accident, cardiac arrest, overdose, etc.) and scheduled non-emergency care like a transport for dialysis, chemotherapy, physical therapy, and the like.

1-252. (canceled)

253. A digital assistance system for automatically capturing emergency medical services (EMS) data to populate an electronic patient care record (ePCR), the system comprising:
a memory storing an ePCR comprising a plurality of data fields, the plurality of data fields comprising at least one first ePCR data field; and
at least one processor configured to execute a digital assistant to
receive input from at least one user interface (UI) device, the input comprising unstructured data corresponding to a human language communication regarding a patient encounter with EMS,
identify, using at least one natural language processor, at least one first ePCR data field corresponding to the unstructured data,
transform, using the at least one natural language processor, at least a portion of the unstructured data to structured data comprising at least one data field value, and
populate the at least one first ePCR data field in the ePCR with the structured data.

254. The digital assistance system of claim 253, wherein the at least one user interface device comprises one or more of a microphone, a touchscreen, a scanner, a camera, a keyboard, and a virtual reality device.

255. The digital assistance system of claim 253, wherein to identify, using at least one natural language processor, the at least one first ePCR data field corresponding to the unstructured data comprises to:
identify at least one intent expressed within the unstructured data to document at least one value of at least one data element defined in an ePCR standard; and
extract at least one slot value from the unstructured data that specifies an identifier of the at least one first ePCR data field.

256. The digital assistance system of claim 255, wherein the digital assistant is configured to
identify at least one second ePCR data field as being procedurally related to the at least one first ePCR data field based on a predictive workflow for the ePCR,
generate at least one prompt associated with the at least one second ePCR data field, and
present the at least one prompt to a caregiver via the at least one UI device.

257. The digital assistance system of claim 256, wherein the digital assistant is configured to
identify a context corresponding to one or more of emergency medical services interventions and procedures for natural language processing based on the unstructured data, and
select the predictive workflow based on the identified context.

258. The digital assistance system of claim 257, wherein the predictive workflow provides an order of population for fields of the ePCR based on one or more of a medical care protocol, historic medical outcomes, and an observed order of population for fields of the ePCR.

259. The digital assistance system of claim 257, wherein the predictive workflow is customizable by an EMS organization.

260. The digital assistance system of claim 256, wherein the at least one prompt comprises a request for input corresponding to at least one second value for the at least one second ePCR data field.

261. The digital assistance system of claim 256, wherein the at least one prompt comprises one or more of an instruction, a reminder, and an alarm corresponding to an intervention associated with the at least one second ePCR data field.

262. The digital assistance system of claim 256, wherein the at least one prompt comprises a request for confirmation of the at least one data field value prior to population of the at least one first ePCR data field.

263. The digital assistance system of claim 256, wherein the at least one first ePCR data field and the at least one second ePCR data field correspond to different sections of the ePCR.

264. The digital assistance system of claim 255,
wherein to identify the at least one intent comprises to generate a metric that indicates a confidence that the at least one intent is at least one actual intent, and
wherein the at least one processor is configured to switch a default interactivity mode of the digital assistant to a confirmation mode in response to the metric being less than a threshold value and to switch the default interactivity mode of the digital assistant to an observational mode in response to the metric being greater than a threshold value.

265. The digital assistance system of claim 255, wherein the at least one intent comprises at least one intent to document one or more of patient data, a task undertaken by a caregiver, or information required by a section of the ePCR.

266. The digital assistance system of claim 255, wherein the at least one natural language processor is trained using textual structures used by EMS caregivers.

267. The digital assistance system of claim 266, wherein the textual structures comprise individual sentences that comprise at least one slot value that specifies identifiers of data elements required by the ePCR and one slot value that specifies values for the data elements.

268. The digital assistance system of claim 266, wherein the textual structures are constructed using data elements defined in a data standard for the ePCR and valid values of the data elements.

269. The digital assistance system of claim 266, wherein the textual structures are specific to a type of EMS medical services.

270. The digital assistance system of claim 255, wherein the at least one natural language processor comprises one or more natural language processors trained using data sourced from one or more of an ePCR standard, historical ePCR records, publicly available historical NEMSIS records, historical dispatch records, historical billing account records, or historical billing claims, a medical device, shorthand terminology, a customer specific vocabulary, a context data set, or a section data set for each section in the ePCR standard.

271. The digital assistance system of claim 253, wherein the at least one natural language processor is configured to transform the at least a portion of the unstructured data to structured text using a locally executed natural language processor configured to convert unstructured text to structured text, the natural language processor being trained to identify, within communications articulated in a human language, data elements defined in an ePCR standard.

272. The digital assistance system of claim 271, wherein the ePCR standard is one or more of a National Emergency Medical Service Information System (NEMSIS) standard or a Fast Healthcare Interoperability Resources (FHIR) standard.

273. The digital assistance system of claim 253, wherein the digital assistant is configured to populate the at least one first ePCR data field in the ePCR via interoperations with one or more processors of a cloud server or an edge server.

274. The digital assistance system of claim 273, wherein the interoperations comprise at least one request for the one or more processors to execute natural language processing.

275. The digital assistance system of claim 273, wherein the edge server is disposed at an emergency transport vehicle or in a medical device carrying case.

276. The digital assistance system of claim 273, wherein the edge server is integrated into a medical device.

277. The digital assistance system of claim 253, wherein the at least one natural language processor is configured to transform the at least a portion of the unstructured data to structured data based on a validation requirement for the at least one first ePCR data field.

278. The digital assistance system of claim 277, wherein the validation requirement corresponds to one or more of a National Emergency Medical Service Information System (NEMSIS) standard or an HL7 Fast Healthcare Interoperability Resources (FHIR) standard.

279. The digital assistance system of claim 277, wherein the validation requirement comprises a rule for one or more required fields in the ePCR and wherein the digital assistant is configured to:
confirm that the one or more required fields comprise data values,
identify unfilled required fields, and
prompt a caregiver to provide the unstructured data for the unfilled required fields.

280. The digital assistance system of claim 255, wherein:
the digital assistant is configured to receive the unstructured data as an audible utterance; and
the at least one natural language processor is configured to
render the audible utterance as text, and
identify the at least one intent expressed within the text.

281. The digital assistance system of claim 255, wherein:
the digital assistant is configured to receive the unstructured data as typed text input; and
the at least one natural language processor is configured to identify the at least one intent expressed within the text input.

282. The digital assistance system of claim 253, wherein the memory, the at least one processor, and at least a portion of the one or more UI devices are disposed in a mobile computing device comprising a smartphone.
